(19) United States
(12) Patent Application Publication
(10) Pub. No.: US 2024/0122449 A1
(43) Pub. Date: Apr. 18, 2024

(54) ACCESS DEVICE

(71) Applicant: Grumpy Innovation, Inc., New York City, NY (US)

(72) Inventors: Sameer SHARMA, Sioux Falls, SD (US); Luke Emerson HESS, Bemidji, MN (US); Emily Marie ROBIDEAU, Larkspur, CO (US); Leonard ZERMAN, Larkspur, CO (US); Anthony ASAAD, Ham Lake, MN (US)

(73) Assignee: Grumpy Innovation, Inc., New York City, NY (US)

(21) Appl. No.: 18/393,424

(22) Filed: Dec. 21, 2023

Related U.S. Application Data
(63) Continuation of application No. PCT/US2022/073243, filed on Jun. 28, 2022.
(60) Provisional application No. 63/202,857, filed on Jun. 28, 2021.

(51) Int. Cl. A61B 1/00; A61B 1/005; A61B 1/012; A61B 1/05

(52) U.S. Cl. CPC A61B 1/00066; A61B 1/00032; A61B 1/00039; A61B 1/00103; A61B 1/00124; A61B 1/00128; A61B 1/0055; A61B 1/0125; A61B 1/05

(57) ABSTRACT

A system is disclosed that can have a first device and a second device. The first device can have a tube, a deflectable section, and a camera. The second device can have a first tube. A stabilizer can be advanceable from the first tube. The second device can have a second tube. A third tube can be advanceable from the second tube. The first device can be an endoscope. The first device can be attachable to the second device via a connector.

### CROSS-REFERENCE TO RELATED APPLICATIONS

This application is a continuation of International Patent Application No. PCT/US2022/073243 filed Jun. 28, 2022, which claims the benefit of priority to U.S. Provisional Application No. 63/202,857 filed Jun. 28, 2021, each of which is incorporated herein by reference in its entirety for all purposes.

### BACKGROUND

### 1. Technical Field

This disclosure relates generally to the inspection of body cavities via direct examination and/or indirect examination, for example, via cameras, radiological guidance, or both, through the abdominal wall or natural stoma. This disclosure relates generally to access devices and methods of using the same, and more particularly to the use of access devices for the examination and/or treatment of body cavities, including, for example, the gastrointestinal tract (e.g., the upper gastrointestinal tract, the lower gastrointestinal tract), the nose, and/or the throat, and to the use of access devices for the examination and/or treatment of tissue and/or obstructions such as the tissue walls of body cavities, the vocal cords, and/or obstructions (e.g., intestinal obstructions, sinus obstructions).

### 2. Background

Body cavities can be visualized and examined directly and/or indirectly. Indirect visualization can include the use of fluoroscopic methods, for example, using a two-dimensional imaging technique or a three-dimensional reconstructive technique.

Examination of body cavities can often be prophylactic such as the surveillance of colorectal cancer or can involve the treatment of emergency conditions such as intestinal obstructions. Colon and rectal cancer surveillance is often carried out in a doctor's office, and the treatment of intestinal obstructions is often carried out in an operating room. In such surveillance and treatment locations, it is often not possible to traverse a dilated small or large intestine due to size constraints. It is often necessary or more advantageous to access the intestine through the abdominal wall to travel the path of least distance to the intestinal blockage location so that the surveillance can be performed or so that the cause of the blockage can be diagnosed and treated more safely and quickly.

A need exists for a device that would permit direct entry of the large intestine, visualization of an intestinal blockage, and treatment of the intestinal blockage through the abdominal wall. Current methods require an operation such as a laparotomy to access the large intestine. It would be advantageous to be able to place a device to visualize the obstruction in a modular fashion to permit decompression of the intestine or other body cavity during the procedure.

A need exists for a device (e.g., the device 100 disclosed herein) that allows for minimally invasive entry of any body cavity to permit anatomy viewing and decompression. Currently, a fixed diameter flexible tube with a camera on one end is used.

A need exists for positional placement of a tip of a device using a selectively applied torque member (e.g., the torque transmitter 110) giving 1:1 torquability. Currently, rotation of an entire assembly around its axis causes less than a 1:1 rotation capability.

A need exists for a modular approach to cannulation allowing for construction of an endoscope in a step-by-step process such that entry of the device though a small incision with subsequent dilations is possible.

A need exists for fully sterile single use materials. Current materials are reusable or reposable in nature.

A need exists for improved devices (e.g., the device 100 disclosed herein) to examine and/or treat small bowel obstruction (SBO). SBO is a common disease. For example, it is estimated that there are 350,000 adhesiolysis operations per year in the US. Surgical management is often required (20-30%). Surgical management has postoperative complications (mortality rate: 3%) and has significant healthcare costs (e.g., 8 days of hospitalization, more than $2.0 billion/year). A need exists for a device (e.g., the device 100 disclosed herein) that is less invasive and less expensive that can be used with acute small bowel obstruction, intermittent small bowel obstruction, or with both. A need exists for a device (e.g., the device 100 disclosed herein) that can combine endoscopic and fluoroscopic approaches to examine and clear obstructions. A need exists for a device (e.g., the device 100 disclosed herein) for distal SBO using combined endoscopy and advanced imaging (e.g., fluoroscopic imaging).

### SUMMARY

Access devices are disclosed. Methods of accessing target sites are disclosed. Methods of using access devices are disclosed. Methods of advancing and retracting stabilizers and/or tubes are disclosed. Methods of assembling and disassembling modular devices are disclosed. Methods of assembling and disassembling modular systems are disclosed. Methods of making access devices are disclosed.

An access device is disclosed. The access device can have a tube, a deflectable section, and a camera.

An access device is disclosed. The access device can have a first tube. A stabilizer can be advanceable from the first tube. The access device can have a second tube. A third tube can be advanceable from the second tube.

A system is disclosed. The system can have a first device and a second device. The first device can have a tube, a deflectable section, and a camera. The second device can have a first tube. A stabilizer can be advanceable from the first tube. The second device can have a second tube. A third tube can be advanceable from the second tube.

A method of articulating a tip of an endoscope is disclosed. The method can include articulating a deflectable section having a first segment, a second segment, and a hinge.

A method of assembling and/or disassembling a system is disclosed. The method can include attaching an endoscope to a connector attached to a first tube and/or a second tube, and/or the method can include detaching the endoscope from the connector attached to the first tube and/or the second tube.

A method of advancing and/or retracting a stabilizer is disclosed. The method can include advancing the stabilizer from a first tube, and/or the method can include retracting the stabilizer into the first tube. The first tube can be removably attached to the outside of a second tube.

### BRIEF SUMMARY OF THE DRAWINGS

The drawings shown and described are exemplary variations and non-limiting Like reference numerals indicate identical or functionally equivalent features throughout.

FIG. 1 illustrates a variation of a device in an assembled state.

FIG. 2 illustrates an exploded view of the device of FIG. 1.

FIG. 3 illustrates a perspective view of the handle in FIG. 1.

FIG. 4 illustrates a variation of a cross-section view of the handle of FIG. 3 through the line 4-4.

FIG. 5 illustrates perspective view of the connector in FIG. 1.

FIG. 6 illustrates a top view of the connector of FIG. 5.

FIG. 7 illustrates a side view of the tip in FIG. 1.

FIG. 8 illustrates a perspective view of the tip of FIG. 7.

FIG. 9 illustrates a variation of the device of FIG. 1.

FIG. 10 illustrates a variation of a close-up view of the device of FIG. 9 at section 10-10.

FIG. 11 illustrates the tip of the device of FIG. 1 in a partially curved configuration.

FIG. 12 illustrates the tip of the device of FIG. 1 in a fully curved configuration.

FIG. 13 illustrates the tip of the device of FIG. 1 in a partially curved configuration.

FIG. 14 illustrates the tip of the device of FIG. 1 in a fully curved configuration.

FIG. 15 illustrates the body of the device of FIG. 1 in a curved configuration.

FIG. 16 illustrates a front perspective view of a variation of a tip of the device of FIG. 1.

FIG. 17 illustrates a top view of the tip of FIG. 16.

FIG. 18 illustrates a side view of the tip of FIG. 16 being attached to the body of the device of FIG. 1.

FIG. 19 illustrates that when the tip of the device of FIG. 1 is at a target site, the tip can be detached from the body of the device.

FIG. 20 illustrates the tip of the device of FIG. 1 deployed at a target site.

FIG. 21 illustrates that a stabilizer can be deployed from the tip of the device of FIG. 1.

FIG. 22 illustrates the stabilizer of FIG. 21 can have the shape shown.

FIG. 23 illustrates a front perspective view of a variation of a camera of the device of FIG. 1.

FIG. 24 illustrates a top view of the camera of FIG. 23.

FIG. 25 illustrates a side view of the camera of FIG. 23 being attached to the tip of the device of FIG. 1.

FIG. 26 illustrates that when the camera of FIG. 23 is at the target site, the camera can be detached from the tip of the device of FIG. 1.

FIG. 27 illustrates the camera of FIG. 23 deployed at a target site.

FIG. 28 illustrates that a stabilizer can be deployed from the camera of the device of FIG. 1.

FIG. 29 illustrates the body, the connector, and the handle of the device of FIG. 1 in a disassembled state.

FIG. 30 illustrates the body, the connector, and the handle of the device of FIG. 1 in an assembled state.

FIG. 31 illustrates a front perspective view of a variation of the handle of the device of FIG. 1.

FIG. 32 illustrates a top view of the handle of FIG. 31.

FIG. 33 illustrates a bottom view of the handle of FIG. 31.

FIGS. 34 and 35 illustrate that the connector of the device of FIG. 1 can be removably connectable to an extension on the handle.

FIGS. 36 and 37 illustrate that the handle can include a waterproof universal serial bus (USB) component that can be removably connected to a module.

FIG. 38 illustrates that the handle can be removably connected to the module of FIG. 37.

FIGS. 39-41 illustrate an exemplary deployment process.

FIG. 42 illustrates that the handle can have a removably connectable module of electronic components.

FIG. 43A-43D illustrate a variation of a PCB schematic for the device of FIG. 1.

FIG. 44 illustrates a variation of a device in an assembled state.

FIG. 45 illustrates a variation of a cover on the handle of the device of FIG. 44.

FIG. 46 illustrates an exploded view of the device of FIG. 44.

FIG. 47 illustrates a rear perspective view of the handle of the device of FIG. 44.

FIG. 48 illustrates a rear perspective view of a module of the device of FIG. 44.

FIG. 49 illustrates a front perspective view of the module of FIG. 48.

FIG. 50 illustrates a rear view of the module of FIG. 48.

FIG. 51 illustrates a side view of the module of FIG. 48.

FIG. 52 illustrates a bottom view of the module of FIG. 48.

FIG. 53 illustrates a side view of the camera and the connector in section 46x in FIG. 46.

FIG. 54 illustrates a front perspective view of the camera and the connector in section 46x in FIG. 46.

FIG. 55 illustrates a front view of the camera of FIGS. 53 and 54.

FIG. 56 illustrates a side view of a variation of the deflectable section of the device of FIG. 44.

FIG. 57 illustrates a perspective view of the deflectable section of FIG. 56.

FIG. 58 illustrates a top view of the deflectable section of FIG. 56.

FIG. 59 illustrates a bottom view of the deflectable section of FIG. 56.

FIG. 60 illustrates the device of FIG. 44 with various components shown transparent.

FIG. 61 illustrates a closeup of section 60x in FIG. 60.

FIG. 62 illustrates a closeup of section 61x in FIG. 61.

FIG. 63 illustrates a perspective view of the deflectable section of FIG. 62.

FIG. 64 illustrates a top view of the deflectable section of FIG. 62.

FIG. 65 illustrates a bottom view of the deflectable section of FIG. 62.

FIG. 66 illustrates a variation of a deflectable section.

FIG. 67a illustrates the deflectable section of FIG. 66 in a curved configuration.

FIG. 67b illustrates the deflectable section of FIG. 66 in a curved configuration.

FIG. 68 illustrates a variation of a ratchet system of the device of FIG. 44.

FIG. 69 illustrates a side view of the ratchet system of FIG. 68 taken along the line 69-69 in FIG. 68.

FIG. 70 illustrates a side view of the ratchet system of FIG. 68 taken along the line 70-70 in FIG. 68.

FIG. 71 illustrates a variation of teeth of the ratchet system of FIG. 68.

FIG. 72 illustrates a variation of a tensioning system.

FIGS. 73 and 74 illustrates that the tensioner of the tensioning system of FIG. 72 can be engaged with a connector of the device.

FIGS. 75 and 76 illustrates that the tensioner of the tensioning system of FIG. 72 can be engaged with a connector of the device.

FIG. 77a illustrates a variation of a first size of the device of FIG. 44.

FIG. 77b illustrates a variation of a second size of the device of FIG. 44.

FIG. 77c illustrates a variation of a second size of the device of FIG. 44.

FIG. 78a illustrates the device of FIG. 44 can be inserted through the nasal cavity.

FIG. 78b illustrates the device of FIG. 44 can be inserted through the mouth.

FIG. 79 illustrates a variation of a device in an assembled state.

FIG. 80 illustrates a variation of a system in an assembled state.

FIG. 81 illustrates a variation of a computer of the system of FIG. 80.

FIG. 82a illustrates a closeup of section 80x in FIG. 80 with the stabilizer of the device of FIG. 79 in a non-deployed configuration and with a tube of the device of FIG. 79 in a non-deployed configuration.

FIG. 82b illustrates a front perspective view of FIG. 82a.

FIG. 83a illustrates a closeup of section 80x in FIG. 80.

FIG. 83b illustrates a front perspective view of FIG. 83a.

FIG. 84a illustrates a closeup of section 80x in FIG. 80 with the stabilizer of the device of FIG. 79 in an advanced configuration and with a tube of the device of FIG. 79 in an advanced configuration.

FIG. 84b illustrates a front perspective view of FIG. 84a.

FIGS. 85a-85d illustrate the stabilizer of the device of FIG. 79 in various stages.

FIGS. 86a-86d illustrate the stabilizer of the device of FIG. 79 in various stages.

FIG. 87a illustrates a variation of the device of FIG. 44.

FIG. 87b illustrates a closeup of section 87ax1 in FIG. 87a.

FIG. 87c illustrates a front perspective view of the camera of FIGS. 87a and 87b.

FIG. 87d illustrates a front view of the camera of FIGS. 87a and 87b.

FIG. 87e illustrates a front perspective view of a variation of the device of FIG. 87a attached to the device of FIG. 79.

FIG. 88a illustrates a top view of a variation of the handle of FIG. 87a in section 87ax2.

FIG. 88b illustrates a side view of the handle of FIG. 88a.

FIG. 88c illustrates a side view of section 88ax in FIG. 88a.

FIG. 88d illustrates a front view of section 88ax in FIG. 88a.

FIG. 89a illustrates a variation of a cover.

FIG. 89b illustrates a variation of a cover.

FIG. 90 illustrates a variation of the module of FIG. 48.

FIG. 91 illustrates a variation of the module of FIG. 48.

FIGS. 92a and 92b illustrate testing data of the device of FIG. 79 and stability testing results.

### DETAILED DESCRIPTION

The features in FIGS. 1-91 and/or the features described herein can be combined with each other in any combination.

FIG. 1 illustrates a variation of an access device 100 (also referred to as the device 100) that can be inserted into body cavities, for example, into lumens of anatomical structures of a person. The lumens can be, for example, part of the person's gastrointestinal anatomy, respiratory anatomy, reproductive anatomy, vascular anatomy, or urinary anatomy.

The device 100 can be a cannulation device, a visualization device (e.g., an endoscope), a tissue engagement device, a deployment device (e.g., tool deployment device, implant deployment device), an implant, a passable implant, a body cavity decompression device, or any combination thereof. The device 100 can examine and/or treat partial obstructions in body cavities. The device 100 can examine and/or treat complete obstructions in body cavities. The device 100 can examine and/or treat partial and complete obstructions in body cavities. For example, the device 100 can be a cannulator that can visualize tissue with an endoscope, for example, to visualize and treat intestinal obstructions in the small and/or large intestines. The device 100 can be used for endoscopic and/or fluoroscopic therapy of body cavity obstructions, for example, small bowel obstructions (e.g., acute small bowel obstruction, intermittent small bowel obstruction), including in the distal ileum, for example, for single band adhesion, for surgical treatment (e.g., strangulation, peritonitis, bowel ischemia), or any combination thereof (e.g., for surgical treatment and/or for endoscopy and fluoroscopy therapy). The device 100 can be, for example, an endoscope.

The device 100 can assist with cannulation, visualization, stabilization, and selective interchange of accessory devices within a torquable member sheath configuration.

FIG. 1 illustrates that the device 100 can have a handle 102, a connector 104, a body 106, a tip 108, a torque transmitter 110, actuators 112, a camera 114, or any combination thereof, each of which can be reusable or disposable. The torque transmitter 110 (also referred to as the torque member, the torque deliverer) the actuators 112, and electronic wires (e.g., that connect the camera 114 to electronics in the handle 102) can extend through lumens 107 in the tip 108, the body 106, the connector 104, and the handle 102, or any combination thereof.

The body 106 can be a sheath. The body 106 can be a tube. The body 106 can be, for example, a flexible sheath or a flexible tube. For example, FIG. 1 illustrates that the body 106 can be a steerable catheter having a lumen 107 for the torque transmitter 110 and a lumen 107 for each of the actuators 112. FIG. 1 illustrates that the lumens 107 for the actuators 112 can be side lumens and that the lumen 107 for the torque transmitter 110 can be a central lumen. The body 106 can be a flexible multi-element extrusion that can house the torque transmitter 110, the actuators 112, and electronic wires (e.g., electronic wire or wires for the camera 114). For example, the body 106 can include a steerable catheter having a polytetrafluoroethylene (PTFE) liner and an inner jacket with braiding, and an outer stiffer jacket that can allow selective inner catheter length steering. The body 106 can have sections with different durometers.

The torque transmitter 110 can transmit torque applied at the proximal end of the device 100 to the distal end of the device 100. The torque transmitter 110 can give a 1:1 force transfer ratio between the proximal end of the device 100 and the distal end of the device 100. The torque transmitter 110 can give a 1:1 torquability ratio between the proximal end of the device 100 and the distal end of the device 100. For example, FIG. 1 illustrates that when a first torque T1 or a second torque T2 is applied to a proximal end of the device 100 (e.g., to the handle 102 and/or to the proximal end of the body 106), the first torque T1 or the second torque T2, respectively, can be transmitted to the distal end of the device 100 (e.g., to the distal end of the body 106 and to the tip 108). A 1:1 torquability ratio can allow the user to rotate the distal end of the device 100 with precision, for example, with the same torque (e.g., the first torque T1 or the second torque T2) that the user applies to proximal end of the of the device 100. The first and second torques T1, T2 can be opposite each other. For example, the first torque T1 can be a counterclockwise torque and the second torque T2 can be a clockwise torque. The first torque T1 can cause the device 100 to rotate in direction 118a and the second torque T2 can cause the device 100 to rotate in direction 118b. Directions 118a and 118b can be opposite each other (e.g., direction 118a can be counterclockwise and direction 118b can be clockwise).

The torque transmitter 110 can be removably positionable in the device 100, for example, in one of the lumens 107 (e.g., in the central lumen in FIG. 1). The torque transmitter 110 can be removably positionable in the body 106. The torque transmitter 110 can be removably positionable in the body 106 and in the tip 108. For example, FIG. 1 illustrates that the torque transmitter 110 can be advanced (e.g., in direction 116a) and retracted (e.g., in direction 116b) in the body 106 and in the tip 108. The torque transmitter 110 can be moved in directions 116a and 116b, for example, by hand and/or with a control on the handle 102. Direction 116a can be a first direction, and direction 116b can be a second direction opposite the first direction. The distal terminal end of the torque transmitter 110 can be positioned anywhere along the body 106, anywhere along the tip 108, or distal the tip 108. When the torque transmitter 110 is positioned in the body 106 (e.g., as shown in FIG. 1) or in the body 106 and in the tip 108, the torque transmitter 110 can allow the body 106 and the tip 108 to be rotated by rotating a proximal end of the device 100 (e.g., the handle 102) in direction 118a or direction 118b.

The device 100 (e.g., a surface defining a lumen 107 in the body 106 and/or in the tip 108) can releasably grip the torque transmitter 110 to inhibit or prevent relative axial and/or rotational movement between the torque transmitter 110, the body 106, and the tip 108 when the proximal end of the device 100 is rotated (e.g., by applying the first torque T1 or the second torque T2 to the handle 102 as shown in FIG. 1).

For example, in the torque gripping sense, a luminal surface (e.g., of one of the lumens 107) in the body 106 and/or in the tip 108 can be coated with a gripping coating and/or can have grippers (e.g., gecko feet). The gripping coating and/or the grippers can grip a member (e.g., the torque transmitter 110) positioned in the lumen 107 (e.g., the central lumen) of the body 106 and the tip 108 when the user torques the whole assembly (e.g., with the first torque T1 or with the second torque T2). The torque transmitter 110 can be removably positionable in the device 100 (e.g., in the body 106 and/or in the tip 108). As another example, the torque transmitter 110 can be integrated into the body 106 (e.g., the connector 200 can be a torque transmitter). As another example, the torque transmitter 110 can be integrated with the body 106.

The surface defining the lumen 107 (also referred to as a luminal surface) that the torque transmitter 110 is positionable in can have a gripper that grips the torque transmitter 110. The gripper can be, for example, a coating and/or extensions (e.g., gecko feet) that extend from the luminal surface. The coating and/or the extensions can provide friction between the torque transmitter 110 and the luminal surface such that when the user torques the proximal end of the device 100 (e.g., with the first torque T1 or with the second torque T2), the torque transmitter 110 can transmit the applied torque to the distal end of the device 100.

The surfaces defining the lumens 107 (also referred to as luminal surfaces) can have a coating but may not have a coating. Some of the luminal surfaces can have a coating and some of the luminal surfaces may not have a coating. Different luminal surfaces can have different coatings. For example, FIG. 1 illustrates that the surfaces defining the lumens 107 that the actuators 112 are in can have a lubricous coating and that the surface of the lumen 107 that the torque transmitter 110 is in can have a gripping coating that can grip the torque transmitter 110.

The device 100 (e.g., the handle 102) can grip the torque transmitter 110. For example, the inside of the handle 102 can have a grabber that can cinch down onto the torque transmitter 110 when the torque transmitter 110 is positioned in the device 100. The grabber can be, for example, a seal (e.g., a passive seal). The grabber can be, for example, a tightening silicone gasket. The grabber can maintain rotational and/or axial locking or “clocking” of the torque transmitter 110 relative to the tip 108 so that torque applied to the proximal end of the device 100 can be transmitted to the distal end of the device 100 via the torque transmitter 110. The grabber can rotationally and/or axially lock the torque transmitter 110 in position relative to the body 106 and the tip 108 so that torque (e.g., the first torque T1 or the second torque T2) applied to the proximal end of the device 100 can be transmitted to the distal end of the device 100, for example, along the torque transmitter 110.

The device 100 (e.g., the body 106 and/or the tip 108) can be more rigid when the torque transmitter 110 is in the device 100 than when the torque transmitter 110 is withdrawn from the device 100. For example, the device 100 (e.g., the body 106 and/or the tip 108) can be more rigid when the torque transmitter 110 is inside a lumen 107 (e.g., inside the central lumen 107 in FIG. 1) than when the torque transmitter 110 is outside the lumen 107 (e.g., outside the central lumen 107 in FIG. 1). The device 100 (e.g., the body 106 and/or the tip 108) can be more flexible when the torque transmitter 110 is withdrawn from the device 100 than when the torque transmitter 110 is in the device 100. For example, the device 100 (e.g., the body 106 and/or the tip 108) can be more flexible when the torque transmitter 110 is outside a lumen 107 (e.g., outside the central lumen 107 in FIG. 1) than when the torque transmitter 110 is inside the lumen 107 (e.g., inside the central lumen 107 in FIG. 1).

The tip 108 can be articulatable with the actuators 112. FIG. 1 illustrates that a distal end of the actuators 112 can be attached to the tip 108 such that when the actuators 112 are pulled, the tip 108 can form various arcs having various radii of curvature. The device 100 can have zero, one, or more actuators 112. For example, FIG. 1 illustrates that the device 100 can have a first actuator 112a and a second actuator 112b, where the first actuator 112a and the second actuator 112b can each be attached to a distal end of the tip 108. When the first actuator 112a is moved (e.g., pulled) in direction 116b, the tip 108 can move in a first direction 120a to form various first arcs. When the second actuator 112b is moved (e.g., pulled) in direction 116b, the tip 108 can move in a second direction 120b to form various second arcs. Directions 120a and 120b can be opposite of each other. The tip 108 can be articulated with the actuators 112, for example, to steer the device 100. The tip 108 can be articulated with the actuators 112, for example, to change a viewing angle of the camera 114.

The device 100 can be steerable by articulating the tip 108 with the actuators 112 and/or by rotating the tip 108 by applying torque T1 or torque T2 at a proximal end of the device 100 when the torque transmitter 110 is positioned in the device 100 (e.g., in the position shown in FIG. 1).

The position of the camera 114 in the body (e.g., at the target site) can be changeable, for example, by articulating the tip 108 with the actuators 112, by rotating the tip 108 by applying torque T1 or torque T2 at a proximal end of the device 100 when the torque transmitter 110 is positioned in the device 100 (e.g., in the position shown in FIG. 1), or by both.

The torque transmitter 110 can be, for example, a rod, a wire, or a braided wire. The actuators 112 can be, for example, a rod, a wire, or a braided wire. For example, FIG. 1 illustrates that the torque transmitter 110 can be a braided wire (e.g., to permit directional force transfer in directions 118a and 118b) and that the actuators 112 can be pullable wires. The torque transmitter 110 can have a uniform width (e.g., diameter) or a variable diameter. For example, the width of the torque transmitter 110 can become progressively thinner or thicker near the tip 108.

FIG. 1 illustrates that the camera 114 can be attached to or integrated with the tip 108. For example, FIG. 1 illustrates that the camera 114 can be attached to or integrated to a distal end of the tip 108. The camera 114 can be a distal terminal end of the tip 108 or can form a portion of a distal end of the tip 108. As another example, the camera 114 can be removably attachable to the tip 108. The camera 114 can have, for example, a field of view from 120 degrees to 170 degrees, including every 1 degree increment within this range (e.g., 120 degrees, 150 degrees, 170 degrees).

FIG. 1 illustrates that the tip 108 can have notches 122 that the tip 108 can flex along. The notches 122 can allow the tip 108 to flex when the actuators 112 are tensioned (e.g., pulled in direction 116b). The notches 122 can thereby assist in articulation of the tip 108. FIG. 1 illustrates that the tip 108 can have the camera 114 and the notches 122. The camera 114 can be positioned anywhere along the tip 108, including, for example, on a proximal end of the tip 108 in a position proximal the notches 122, between two notches 122, in a notch 122, or on a distal end of the tip 108 in a position distal the notches 122. For example, FIG. 1 illustrates that the camera 114 can be attached to or integrated to a distal end of the tip 108 in a position distal the notches 122.

FIG. 1 illustrates that the device 100 can have suture holders 124, for example, four suture holders 124 arranged around the perimeter of the handle 102. The suture holders 124 can be arranged around the perimeter of the handle 102, for example, every 90 degrees such that three of the suture holders 124 are visible in FIG. 1 and one of the suture holders 124 is obstructed from view in FIG. 1. Suture can be housed (e.g., temporarily housed) in the suture holders 124 during a procedure. Any length of suture above the minimum needed to tie a knot can be housed in the suture holders 124. The suture can be used to fix the device 100 (e.g., the handle 102) to the patient to prevent slippage of the device 100, which can enable the doctor to free up a hand to perform other tasks.

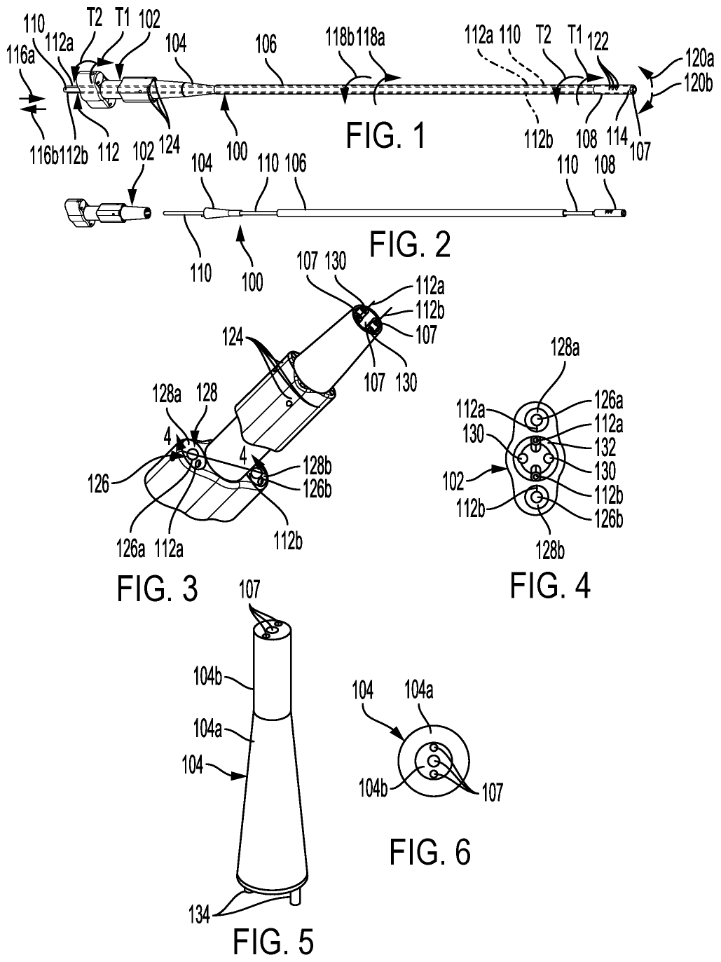

FIG. 2 illustrates that the device 100 in FIG. 1 can be modular, with FIG. 1 illustrating the modular components in an assembled configuration. For example, FIG. 2 illustrates that the handle 102 can be removably attachable to the connector 104, that the connector 104 can be removably attachable to the body 106, and that the body 106 can be removably attachable to the tip 108. These components can be removably connectable, for example, with snap fits, clip fits, screw fits, friction fits, or any combination thereof. The actuators 112 are shown transparent in FIG. 2 for illustrative purposes. The device 100 can be disposable.

FIG. 2 illustrates that the handle 102, the connector 104, the body 106, the tip 108, the torque transmitter 110, and the actuators 112, or any combination thereof can be modular and replaceable before, during, and/or after use. The handle 102, the connector 104, the body 106, the tip 108, the torque transmitter 110, and the actuators 112, or any combination thereof can be removed from the device 100 at any given time, for example, before use, during use, and/or after use.

The actuators 112 can be controlled with one or multiple controls 126 (also referred to as the control 126 or the controls 126), for example, 1 to 6 controls, including every 1 control increment within this range (e.g., 1 control, 2 controls, 3 controls, 4 controls, 5 controls, 6 controls). FIG. 3 illustrates that the handle 102 can have the controls 126. FIG. 3 illustrates that the controls 126 can include, for example, a first control 126a and a second control 126b. The controls 126 can be housed in channels 128 (e.g., in first and second channels 128a, 128b). The channels 128 can be channels in the handle 102. The actuators 112 can be attached to the controls 126. For example, a proximal end of the actuators 112 can be attached to the controls 126 (e.g., instead of extending through the handle 102 as shown in FIG. 1) and a distal end of the actuators 112 can be attached to the tip 108. For example, FIG. 3 illustrates that a proximal end of the first actuator 112a can be attached to the first control 126a and that a proximal end of the second actuator 112b can be attached to the second control 126b. The controls 126 can be, for example, switches, levers, knobs, wheels, or any combination thereof. The controls 126 can be translated (e.g., pulled, pushed, slid), rotated, or translated and rotated to tension and de-tension the actuators 112 to control the amount of deflection in the tip 108. FIG. 3 illustrates that the actuators 112 can extend through an opening in a wall of the channels 128 and into the lumens 107. As another example, the handle 102 can have one control 126 (e.g., a single control 126). As yet another example, the handle 102 may not have any controls 126 (e.g., the handle may have zero controls 126), whereby a user can pull on the actuators 112 directly with their hands to control the amount of deflection in the tip 108. For example, FIG. 1 illustrates that a user can pull on the actuators 112 directly with their hands.

When the first control 126a is rotated in a first direction, the first control 126a can tension (e.g., pull) the first actuator 112a in direction 116b shown in FIG. 1. The tension applied to the first actuator 112a can be released, for example, by releasing the first control 126a. When the first control 126a is released, the first control 126a can rotate in a second direction opposite the first direction to return to a neutral position (e.g., to the position shown in FIG. 3), and thereby release the tension applied to the first actuator 112a.

When the second control 126b is rotated in a first direction, the second control 126b can tension (e.g., pull) the second actuator 112b in direction 116b shown in FIG. 1. The tension applied to the second actuator 112b can be released, for example, by releasing the second control 126b. When the second control 126b is released, the second control 126b can rotate in a second direction opposite the first direction to return to a neutral position (e.g., to the position shown in FIG. 3), and thereby release the tension applied to the second actuator 112b.

As another example, the handle 102 can have one control 126 for the actuators 112 (e.g., a rotatable knob or a rotatable wheel), such that when the control 126 is rotated in a first direction, the control 126 can tension (e.g., pull) the first actuator 112a in direction 116b shown in FIG. 1, and such that such that when the control 126 is rotated in a second direction opposite the first direction, the control 126 can tension (e.g., pull) the second actuator 112b in direction 116b shown in FIG. 1.

FIG. 3 illustrates that the lumens 107 can extend through the handle 102 and that the actuators 112 can be in two lumens 107 and that the torque transmitter 110 can be removably insertable into the lumen 107. FIG. 3 illustrates, for example, that the handle 102 can have three lumens 107, two of which can be side lumens, and one of which can be a central lumen. For example, FIG. 3 illustrates that the lumens 107 that the actuators 112 can extend through can be side lumens and that the lumen 107 that the lumen 107 that the torque transmitter 110 can extend through can be a central lumen.

FIG. 3 illustrates that the handle 102 can have ports 130 that the connector 104 can connect to. For example, the connector 104 can have extensions (e.g., pegs, rods, legs) that can fit into the ports 130 with a friction fit, snap fit, magnetic fit, or any combination thereof.

FIG. 4 illustrates that the lumens 107 for the actuators 112 can be in a wall 132 of the handle 102. FIG. 4 illustrates that an outer surface of the wall of the lumens 107 for the actuators 112 can define the lumen 107 for the torque transmitter 110.

FIG. 5 illustrates that the connector 104 can have extensions 134 that can fit into the ports 130 of the handle 102, for example, with friction fit, snap fit, magnetic fit, or any combination thereof. The extensions 134 can be, for example, pegs, rods, or legs. The extensions 134 (e.g., pegs) can allow the connector 104 to be releasably secured to the handle 102.

FIG. 5 illustrates that the connector 104 can have a connector first end 104a and a connector second end 104b. A proximal end of the connector first end 104a can be releasably connected to the handle 102. A distal end of the connector first end 104a and/or the connector second end 104b can be releasably connected to the body 106. For example, a proximal end of the body 106 can be slidable over the connector second end 104b (e.g., with or without a friction fit). As another example, the proximal end of the body 106 can be slidable over the connector second end 104b (e.g., with or without a friction fit) and the proximal end of the body 106 can be slidable over the distal end of the connector first end 104a (e.g., with a friction fit).

FIG. 5 illustrates that the connector 104 can be a transition piece that can removably connect the handle 102 and the body 106. The connector 104 can be, for example, a hollow cone portion that leads to a multi-lumen segment that can be seen in FIG. 5. For example, the connector first end 104a can have frustoconical shape and the connector second end 104b can have a cylindrical shape. Such an arrangement can allow the proximal end of the body 106 to extend over the connector 104 far enough to result in a releasably secure connection, for example, the releasably secure connection shown in FIG. 1.

FIG. 6 illustrates a top view of FIG. 5.

FIGS. 5 and 6 illustrate three lumens 107, two side lumens 107 and a central lumen 107. The torque transmitter 110 can extend through the central lumen 107 and each one of the actuators 112 can extend through one of the side lumens 107.

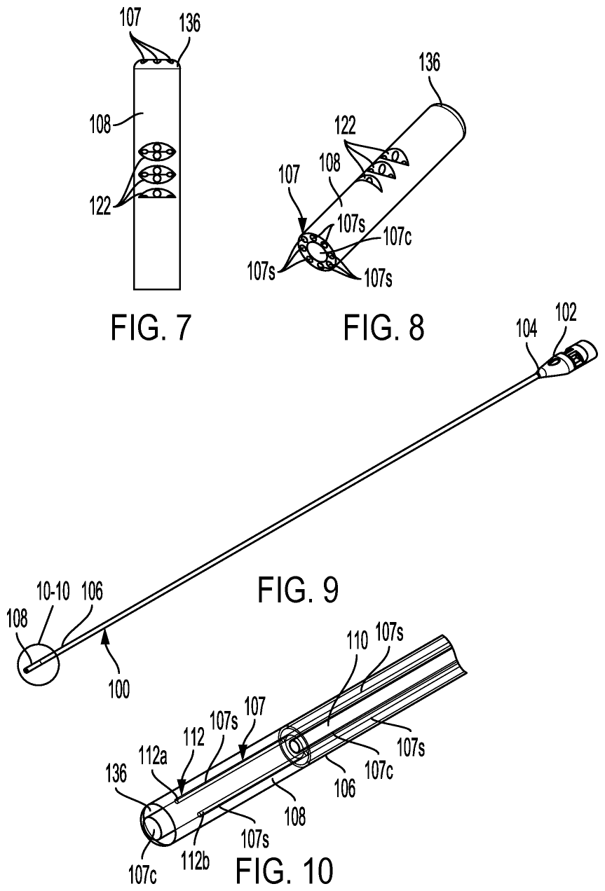

FIG. 7 illustrates that the tip 108 can be flexible, for example, as represented by the notches 122. For example, the tip 108 can be flexible along the length of the tip 108 but the tip 108 can have flexible regions (e.g., notches 122) that the tip 108 can flex along and/or that are more flexible than regions between the flexible regions.

FIG. 7 illustrates that the distal end of the tip 108 (also referred to as the tip distal end 136) can be atraumatic and that the lumens 107 can pass through the tip 108.

FIG. 8 illustrates that the lumens 107 can include side lumens 107s and a central lumen 107c. The side lumens 107s can extend through a wall of each of the components of the device 100 (e.g., through the tip 108, through the body 106, through the connector 104, through the handle 102, or any combination thereof). The central lumen 107c can extend through a center of each of the components of the device 100 (e.g., through the tip 108, through the body 106, through the connector 104, through the handle 102, or any combination thereof). For example, the lumens 107 shown in FIG. 8 can extend through the handle 102, the connector 104, the body 106, and the tip 108, or any combination thereof. FIG. 8 illustrates, for example, that the device 100 can have 8 side lumens 107s and one central lumen 107c. As another example, FIGS. 1-6 illustrate that the device 100 can have two side lumen 107s and one central lumen 107c.

FIGS. 7 and 8 illustrate that some of the lumens 107 (e.g., some of the side lumens 107s) can be exposed by the notches 122.

FIG. 9 illustrates that the tip 108 may not have notches 122.

FIG. 10 illustrates that the distal end of the first actuator 112a can be attached (e.g., fixedly or removably attached) to the tip 108 in a side lumen 107s that can extend through the body 106 and the tip 108.

FIG. 10 illustrates that the distal end of the second actuator 112b can be attached (e.g., fixedly or removably attached) to the tip 108 in a side lumen 107s that can extend through the body 106 and the tip 108.

FIG. 10 illustrates that the side lumens 107s (e.g., the side lumens 107s shown in FIGS. 9 and 10) can extend partially through the tip 108 and that the central lumen 107c can extend completely through the tip 108. FIG. 10 illustrates that the side lumens 107s (e.g., the side lumens 107s shown in FIGS. 9 and 10) may not extend through the tip distal end 136 but that the central lumen 107c can extend through the tip distal end 136.

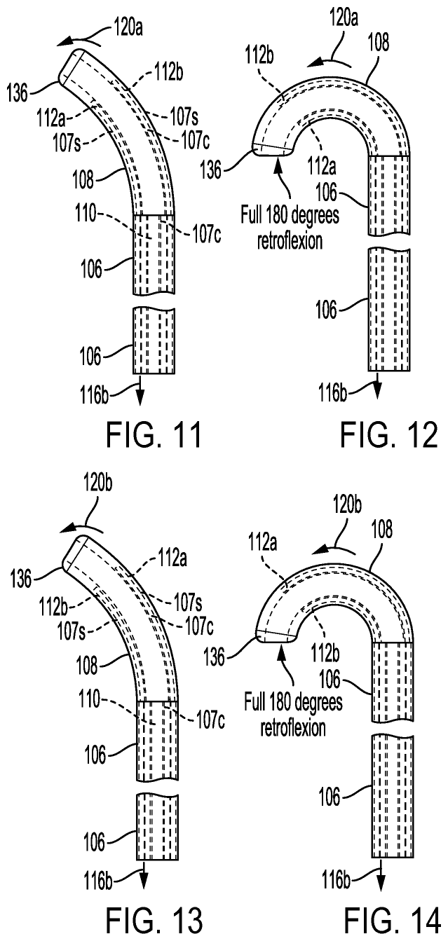

FIGS. 11 and 12 illustrate that when the first actuator 112a is tensioned (e.g., pulled) in direction 116b, for example, by hand or with a control 126 (e.g., with the first control 126a), the tip 108 can move in a first direction 120a to form various first arcs. For example, FIG. 11 illustrates the tip 108 in a partially curved configuration and FIG. 12 illustrates the tip 108 in a fully curved configuration. FIGS. 1, 9, and 10 illustrate an exemplary non-actuated configuration (e.g., straight configuration). When the first actuator 112a is not tensioned (e.g., not actuated), the tip 108 can be straight or non-curved.

FIGS. 1 and 9-12 illustrate that the tip 108 can be deflected from a non-actuated configuration (e.g., the straight configuration in FIGS. 1, 9, and 10) to a fully curved configuration (e.g., the fully curved configuration in FIG. 12) and to any partially curved configuration (e.g., the partially curved configuration in FIG. 11) between the non-actuated configuration and the fully curved configuration. For example, FIGS. 1 and 9-12 illustrate that the tip distal end 136 can be pulled, for example, from 0 degrees (e.g., from the straight configuration in FIGS. 1, 9, and 10) to 180 degrees (e.g., to the fully curved configuration shown in FIG. 12), including every 1 degree increment within this range (e.g., the partially curved configuration shown in FIG. 11). FIG. 12 illustrates that the first actuator 112a can be pulled in direction 116b to cause the tip 108 to assume a full 180 degree retroflexion such that the distal opening of the central lumen 107c points toward the handle 102. When the tension on the first actuator 112a is released, the tip 108 can become less curved (e.g., by moving from the fully curved configuration in FIG. 12 to the partially curved configuration in FIG. 11) or return to the non-actuated configuration. As another example, the tip distal end 136 can be pulled, for example, from 0 degrees (e.g., from the straight configuration in FIGS. 1, 9, and 10) to 270 degrees, for example, 1 degree to 90 degrees past the full 180 degree retroflexion shown in FIG. 12, including every 1 degree increment within this range.

FIGS. 13 and 14 illustrate that when the second actuator 112b is tensioned (e.g., pulled) in direction 116b, for example, by hand or with a control 126 (e.g., with the second control 126b), the tip 108 can move in a second direction 120b to form various first arcs. For example, FIG. 13 illustrates the tip 108 in a partially curved configuration and FIG. 14 illustrates the tip 108 in a fully curved configuration. FIGS. 1, 9, and 10 illustrate an exemplary non-actuated configuration (e.g., straight configuration). When the second actuator 112b is not tensioned (e.g., not actuated), the tip 108 can be straight or non-curved.

FIGS. 1, 9, 10, 13, and 14 illustrate that the tip 108 can be deflected from a non-actuated configuration (e.g., the straight configuration in FIGS. 1, 9, and 10) to a fully curved configuration (e.g., the fully curved configuration in FIG. 14) and to any partially curved configuration (e.g., the partially curved configuration in FIG. 13) between the non-actuated configuration and the fully curved configuration. For example, FIGS. 1, 9, 10, 13, and 14 illustrate that the tip distal end 136 can be pulled, for example, from 0 degrees (e.g., from the straight configuration in FIGS. 1, 9, and 10) to 180 degrees (e.g., to the fully curved configuration shown in FIG. 14), including every 1 degree increment within this range (e.g., the partially curved configuration shown in FIG. 13). FIG. 14 illustrates that the second actuator 112b can be pulled in direction 116b to cause the tip 108 to assume a full 180 degree retroflexion such that the distal opening of the central lumen 107c points toward the handle 102. When the tension on the second actuator 112b is released, the tip 108 can become less curved (e.g., by moving from the fully curved configuration in FIG. 14 to the partially curved configuration in FIG. 13) or return to the non-actuated configuration. As another example, the tip distal end 136 can be pulled, for example, from 0 degrees (e.g., from the straight configuration in FIGS. 1, 9, and 10) to 270 degrees, for example, 1 degree to 90 degrees past the full 180 degree retroflexion shown in FIG. 14, including every 1 degree increment within this range.

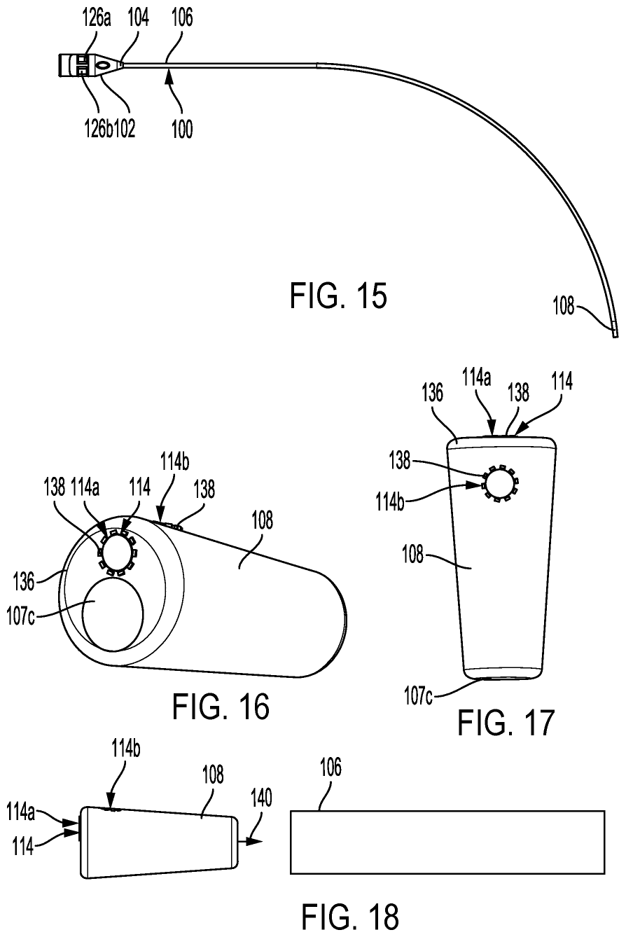

FIG. 15 illustrates that the body 106 can be flexible. For example, the body 106 can be bendable such that the tip 108 can be navigated to a target site by inserting the device 100 over a guidewire. As the device 100 in inserted over the guidewire, the body 106 and/or the tip 108 can bend as the body 106 and the tip 108 are advanced over the guidewire. FIG. 15 illustrates an exemplary bend in the body 106 that the body 106 can assume when the device 100 is advanced over a guidewire. FIG. 15 illustrates, for example, that the body 106 can have a 4 inch to 8 inch bend radius, including every 0.25 inch increment within this range. This can permit the body 106 to be navigated along tortuous anatomy.

FIGS. 16 and 17 illustrate that the tip 108 can have multiple cameras 114, for example, a first camera 114a and/or a second camera 114b. The first camera 114a can be a forward viewing camera and the second camera 114b can be a side viewing camera. The first camera 114a can have, for example, a field of view from 120 degrees to 170 degrees, including every 1 degree increment within this range (e.g., 120 degrees, 150 degrees, 170 degrees). The second camera 114b can have, for example, a field of view from 120 degrees to 170 degrees, including every 1 degree increment within this range (e.g., 120 degrees, 150 degrees, 170 degrees). FIGS. 16 and 17 illustrate that the fields of view of the first and second cameras 114a, 114b can overlap. As another example, the fields of view of the first and second cameras 114a, 114b may not overlap.

The camera 114 (e.g., the first and second cameras 114a, 114b) can have zero, one, or multiple illuminators 138 (also referred to as the illuminator 138 and the illuminators 138) that can illuminate the body cavity. The illuminators 138 can be, for example, lights or light-emitting diodes (LEDs). The camera 114 (e.g., the first and second cameras 114a, 114b) can have, for example, 0-10 illuminators 138, including every 1 illuminator increment within this range (e.g., 0 illuminators, 1 illuminator, 10 illuminators). For example, FIG. 16 illustrates that the first camera 114a can have 10 illuminators 138, and FIG. 17 illustrates that the second camera 114b can have 10 illuminators 138. The first camera 114a can have the same number or a different number of illuminators 138 as the second camera 114b. For example, FIGS. 16 and 17 illustrate that the first camera 114a can each have the same number of illuminators 138 (e.g., 10 illuminators 138) as the second camera 114b.

FIG. 16 illustrates that the central lumen 107c in the tip 108 can be offset from a longitudinal center of the tip 108. As another example, FIGS. 16 and 17 illustrate that the distal opening of the central lumen 107c can be offset from the longitudinal center of the tip 108 and that the proximal opening of the central lumen 107c can be centered with the longitudinal center of the tip 108.

FIGS. 16 and 17 illustrate that the tip 108 can be tapered. For example, FIGS. 16 and 17 illustrate that the tip 108 can have a frustoconical shape.

FIG. 18 illustrates that the taper of the tip 108 shown in FIGS. 16 and 17 can allow the proximal end of the tip 108 to be inserted into the distal end of the body 106 to releasably attach the tip 108 to the body 106. Inserting tip 108 into the body 106 (e.g., as shown by arrow 140) can result in a friction fit, a snap fit, or both between the tip 108 and the body 106. The tip 108 can thereby be releasably attached to the body 106.

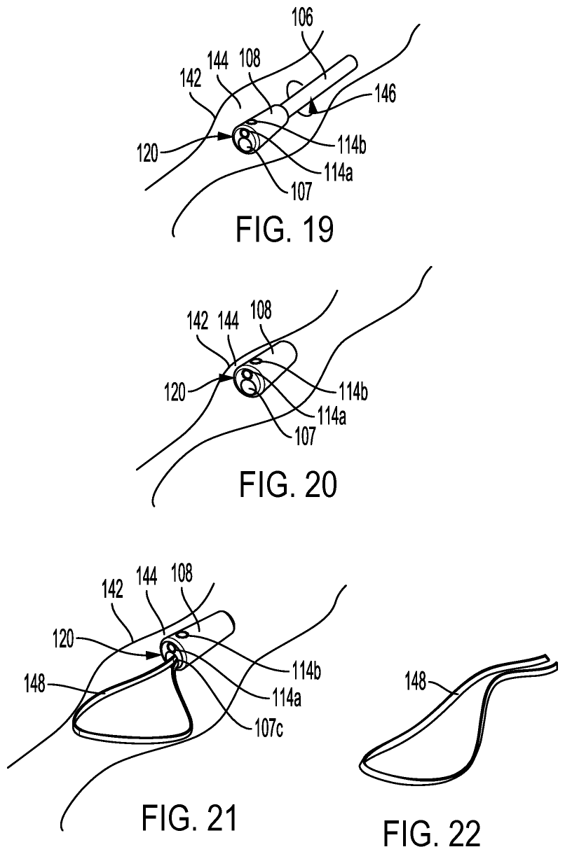

FIGS. 19 and 20 illustrate that the first camera 114a and/or the second camera 114b can be wireless cameras.

FIGS. 19 and 20 illustrate that the tip 108 can be deployable in a body cavity 142 at a target site 144. For example, FIG. 19 illustrates that when the tip 108 is at the target site 144, the tip 108 can be detached from the body 106, for example, by rotating the body 106 as shown by arrow 146, by pushing the tip 108 off the body 106 in a direction opposite to arrow 140 shown in FIG. 18, by unclipping the tip 108 from the body 106, or any combination thereof. FIG. 20 illustrates the tip 108 deployed at the target site 144 and the body 106 withdrawn from the body cavity 142. Once deployed in the body cavity 142 (e.g., at the target site 144), the tip 108 can travel through the body cavity 142. For example, when the body cavity 142 is the gastrointestinal tract, the tip 108 can travel though the gastrointestinal tract, for example, similar to a wireless capsule device, capturing images of the body cavity 142 with the first camera 114a and/or with the second camera 114b as the tip 108 is moved through the gastrointestinal tract by peristalsis.

FIG. 21 illustrates that a stabilizer 148 can be deployable from the tip 108. As another example, the stabilizer 148 may not be deployable from the tip 108 but can instead be attached to the tip 108 (e.g., attached as shown in FIG. 21). The stabilizer 148 can, for example, decompress the body cavity 142 (e.g., compare the compressed body cavity 142 distal of the tip 108 in FIGS. 19 and 20 to the decompressed body cavity 142 distal of the tip 108 in FIG. 21). Such decompression can be helpful to clear obstructions (e.g., intestinal obstructions) in the body cavity 142 (e.g., in the small and/or large intestine). The stabilizer 148 can be deployed from the tip 108 (e.g., extendible and/or retractable from the tip 108), for example, to decompress the body cavity 142. The stabilizer 148 can be an extension or a strip that can extend from the tip 108. The stabilizer 148 can stabilize and/or manipulate (e.g., move) tissue. The stabilizer 148 can be, for example, a Mobius strip (e.g., a Nitinol Mobius strip) that can provide variable stability of the tip 108 as well as tissue manipulation. The stabilizer 148 can thereby provide stabilization for the tip 108 in the body cavity 142. For deployable variations, FIG. 21 illustrates that the stabilizer 148 can be deployable from the central lumen 107c. In such variations, the stabilizer 148 can be extendible and retractable from the tip 108, for example, from the one or more of the lumens 107 (e.g., from the central lumen 107c) via one or more controls on the handle 102 or on another handle. For non-deployable variations, FIG. 21 illustrates that the stabilizer 148 can extend from the central lumen 107c. FIG. 21 illustrates that the stabilizer 148 can have two ends, each of which can be attached to the tip 108 (e.g., to an inside of the tip 108). The stabilizer 148 can stabilize tissue and/or tension tissue. The stabilizer 148 can be a cage, for example, a deployable cage, an expandable cage, and/or a contractible cage. The stabilizer 148 can stabilize tissue. As another example, the stabilizer may not stabilize tissue.

FIG. 22 illustrates the stabilizer 148 in FIG. 21 can have the shape shown in FIG. 22.

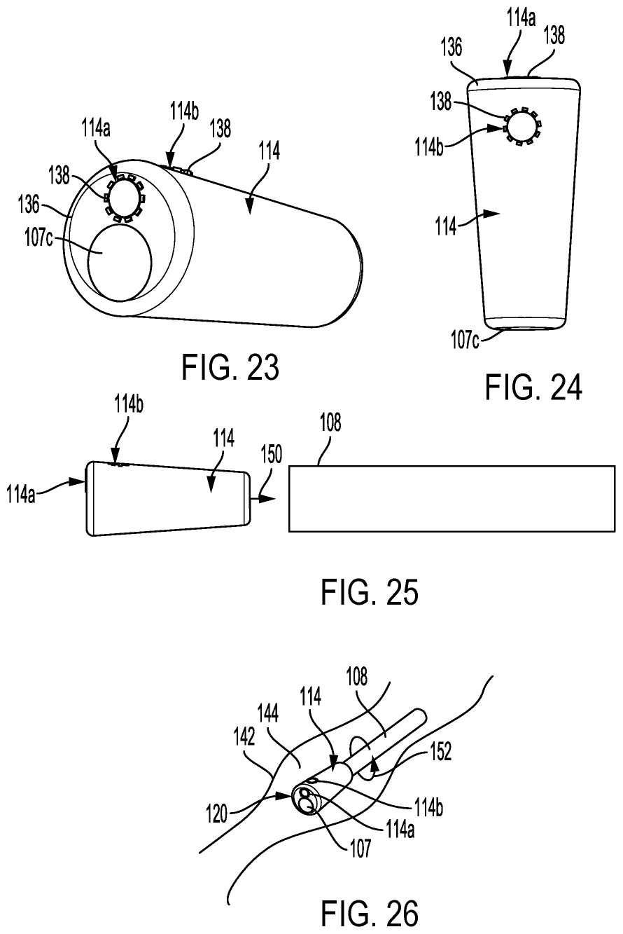

FIGS. 23 and 24 illustrate that the device 100 can have one or multiple cameras 114, for example, the first camera 114a and/or the second camera 114b that can be removably attachable to the tip 108. For example, FIG. 25 illustrates that the camera 114 can be removably attachable to the tip 108. FIGS. 23 and 24 illustrate that the camera 114 can be tapered. For example, FIGS. 23 and 24 illustrate that the camera 114 can have a frustoconical shape. FIG. 25 illustrates that the taper of the camera 114 shown in FIGS. 23 and 24 can allow the proximal end of the camera 114 to be inserted into the distal end of the tip 108 (e.g., into a lumen 107) to releasably attach the camera 114 to the tip 108. Inserting the camera 114 into the tip 108 (e.g., as shown by arrow 150) can result in a friction fit, a snap fit, or both between the camera 114 and the tip 108. A camera 114 can thereby be releasably attached to the tip 108.

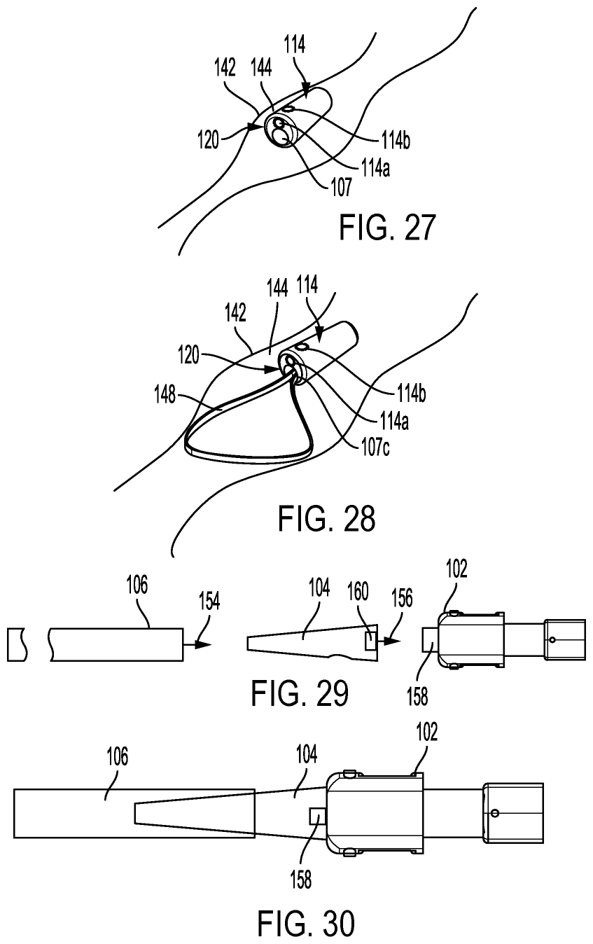

FIGS. 26 and 27 illustrate that the camera 114 can be deployable in the body cavity 142 at the target site 144. For example, FIG. 26 illustrates that when the camera 114 is at the target site 144, the camera 114 can be detached from the tip 108, for example, by rotating the tip 108 as shown by arrow 152, by pushing the camera 114 off the tip 108 in a direction opposite to arrow 150 shown in FIG. 25, by unclipping the camera 114 from the tip 108, or any combination thereof. FIG. 27 illustrates the camera 114 deployed at the target site 144 and the tip 108 withdrawn from the body cavity 142. Once deployed in the body cavity 142 (e.g., at the target site 144), the camera 114 can travel through the body cavity 142. For example, when the body cavity 142 is the gastrointestinal tract, the camera 114 can travel though the gastrointestinal tract, for example, similar to a wireless capsule device, capturing images of the body cavity 142 with the first camera 114a and/or with the second camera 114b as the camera 114 is moved through the gastrointestinal tract by peristalsis.

FIG. 28 illustrates that the stabilizer 148 can be deployable from the camera 114. As another example, the stabilizer 148 may not be deployable from the camera 114 but can instead be attached to the camera 114 (e.g., attached as shown in FIG. 28). The stabilizer 148 can be deployed from the camera 114, for example, to decompress the body cavity 142. The stabilizer 148 can be an extension or a strip that can extend from the camera 114. The stabilizer 148 can stabilize the camera 114 in the body cavity 142. For deployable variations, FIG. 28 illustrates that the stabilizer 148 can be deployable from the central lumen 107c. In such variations, the stabilizer 148 can be extendible and retractable from the camera 114, for example, from the one or more of the lumens 107 (e.g., from the central lumen 107c) via one or more controls on the handle 102 or on another handle. For non-deployable variations, FIG. 28 illustrates that the stabilizer 148 can extend from the central lumen 107c. FIG. 28 illustrates that the stabilizer 148 can have two ends, each of which can be attached to the camera 114 (e.g., to an inside of the camera 114).

FIG. 29 illustrates that the connector 104 can be tapered. For example, FIG. 29 illustrates that the connector 104 can have a frustoconical shape. The taper of the connector 104 can allow the body 106 and the connector 104 to be removably connectable with a friction fit. For example, FIGS. 29 and 30 illustrate that that the proximal end of the body 106 and the distal end of the connector 104 can be removably connectable with a friction fit by inserting the body 106 over the connector 104 (e.g., as shown by arrow 154). The connector 106 is shown transparent in FIG. 30 so that the friction fit can be seen.

FIG. 29 illustrates that the handle 102 can have a clip 158 and that the connector 104 can have a locking slot 160, or vice versa. The body 106 can be releasably locked to the handle 102 via the connector 104 by removably clipping the handle 102 to the connector 104. For example, FIGS. 29 and 30 illustrate that that the clip 158 can be removably clipped to the locking slot 160 (e.g., as shown by arrow 156). To lock the body 106 to the handle 102, the connector 104 can have locking slots that can slide and engage with the clips 158 on the handle 102. When the clip 158 is engaged to the locking slot 160, rotating the handle 102 can rotate the body 106 and the tip 108. When the clip 158 is engaged with the locking slot 160, the handle 102 can torque the camera 114. When the clip 158 is disengaged from the locking slot 160, the handle 102 may not rotate the body 106 and the tip 108.

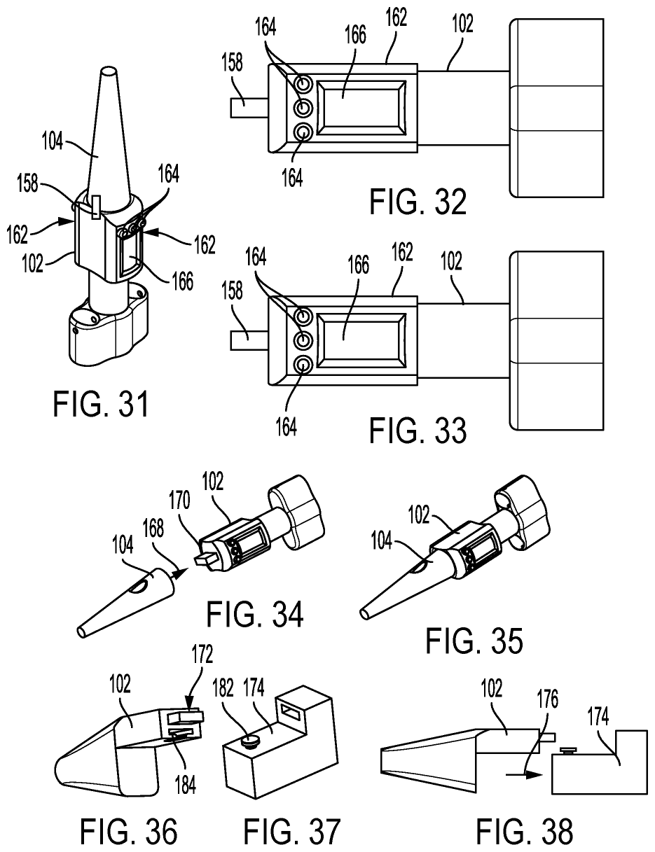

FIG. 31 illustrates a perspective view of FIG. 30 without the body 106 attached to the connector 104.

FIG. 31 illustrates that the handle 102 can have a user interface 162. The user interface 162 can have controls 164 (e.g., buttons) and an electronic display 166. The controls 164 can control the camera 114. The controls 164 can turn the camera 114 on, turn the camera 114 off, can control the illuminators (e.g., on, off, white balance, color, brightness). The display 166 can be, for example, a light-emitting diode (LED) screen or an organic light-emitting diode (OLED) screen. The display 166 can, for example, provide status feedback. The handle 102 can have connections for water instillation. The handle 102 can have controls for the stabilizer 148 (e.g., to extend and retract the stabilizer 148). The display 166 can show the user images of the camera 114 and/or other visualizations of the body cavity that the device is in (e.g., see FIGS. 40 and 41) in real time, for example, images of captured by the camera 114, images captured by a fluoroscopic imaging technique, or both, that can allow the user to view the body cavity and location of the device 100.

FIG. 31 illustrates that the handle 102 can have, for example, two user interfaces 162, on opposite sides of the handle 102 that are identical to each other so that the user interface 162 can be easily accessed, viewed, and controlled as the handle 102 is rotated and moved into different positions. For example, FIG. 32 illustrates a user interface 162 on a first side of the handle 102 in FIG. 31 and FIG. 33 illustrates a user interface 162 on a second side of the handle 102 in FIG. 31. As another example, the two user interfaces 162 can be different from each other.

FIGS. 34 and 35 illustrate that the connector 104 can be removably connectable (e.g., see arrow 168) to an extension 170 of the handle 102, for example, with a snap fit and/or with a friction fit.

FIGS. 36 and 37 illustrate that the handle 102 can include a waterproof universal serial bus (USB) component 172 (e.g., FIG. 36) that can be removably connected to a module 174 (e.g., FIG. 37). FIG. 38 illustrates that the handle 102 can be removably connected to the module 174 (e.g., see arrow 176), for example, by sliding an extension 182 on the module 174 into a slot 184 in the handle 102. The module 174 can be, for example, an electronics module and/or a battery module.

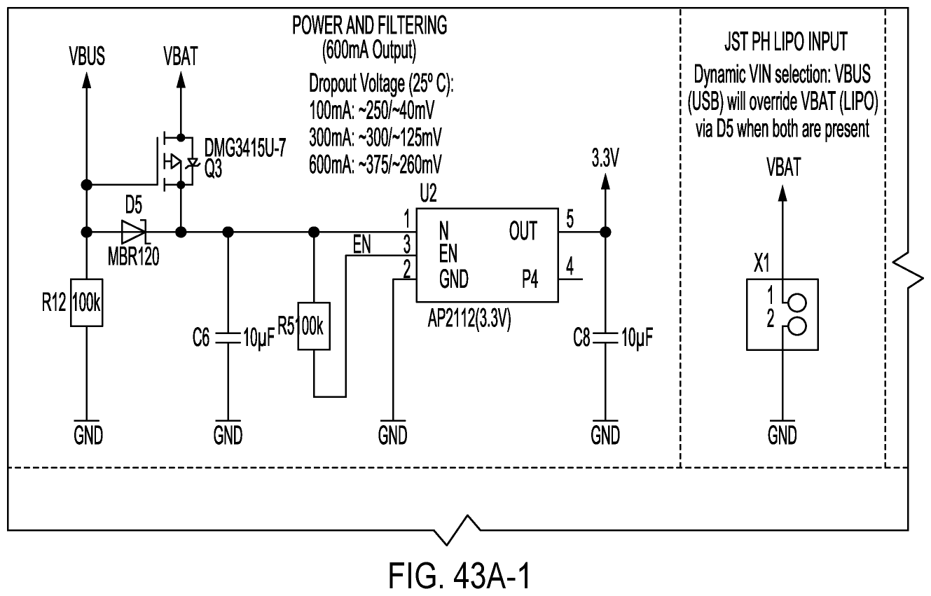

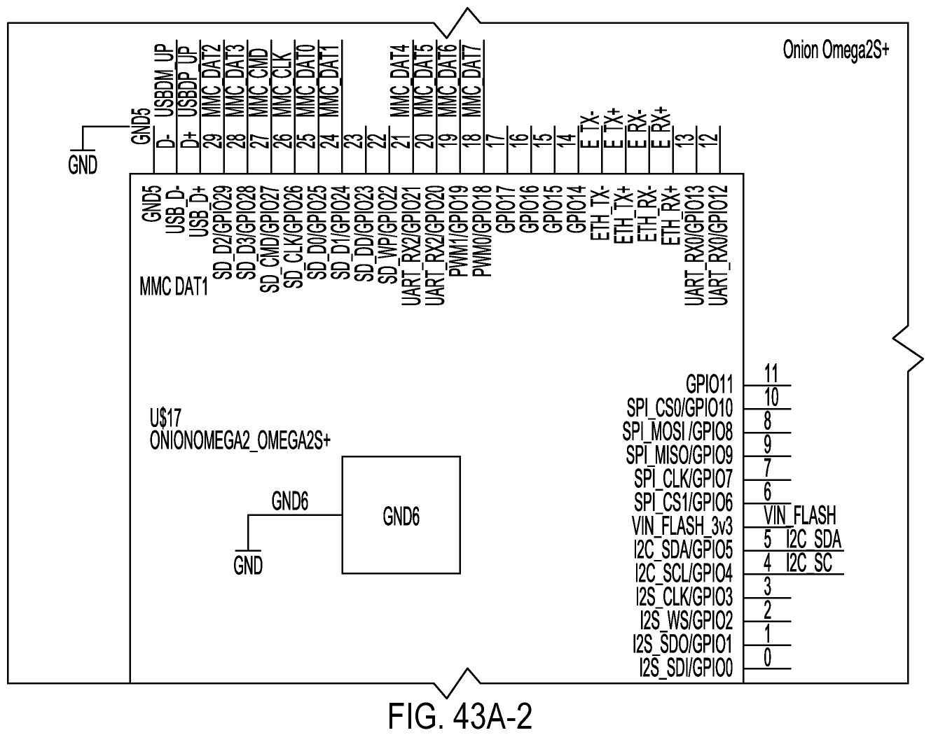

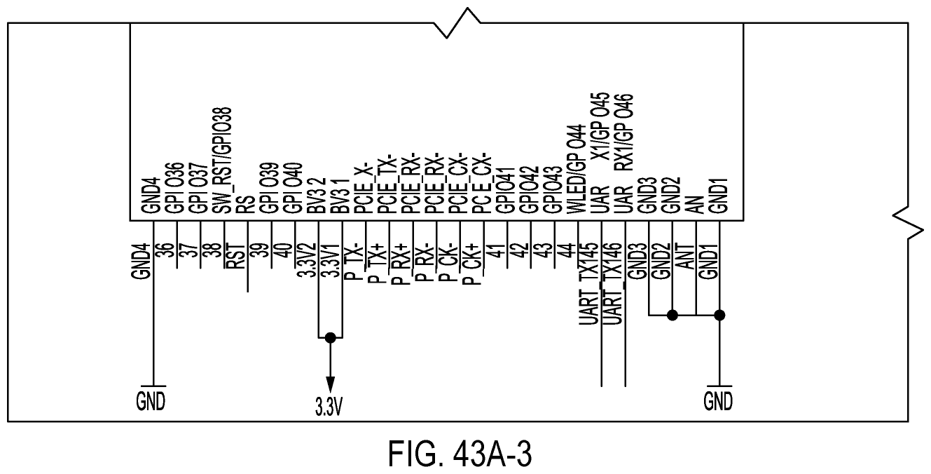

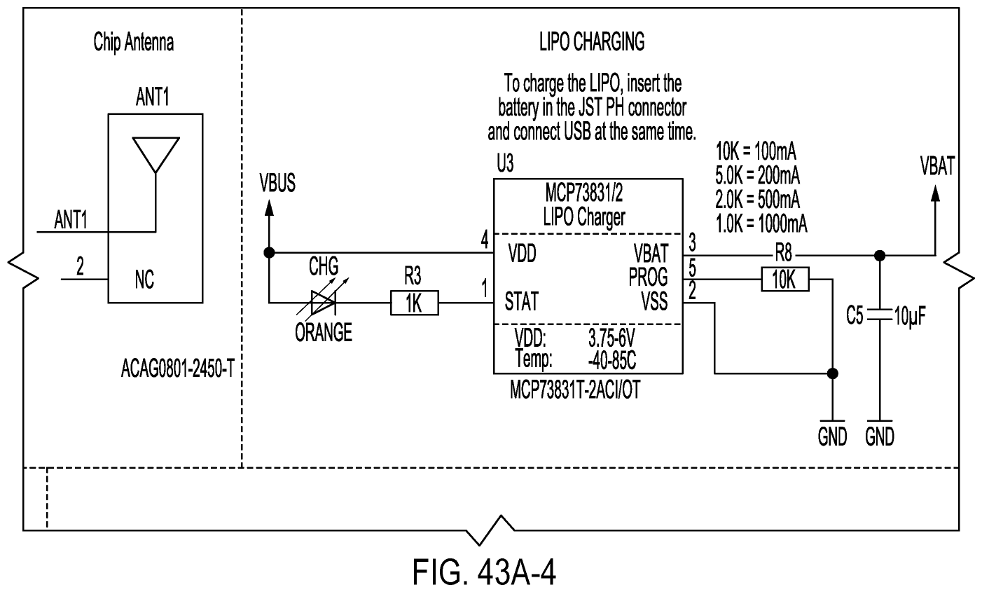

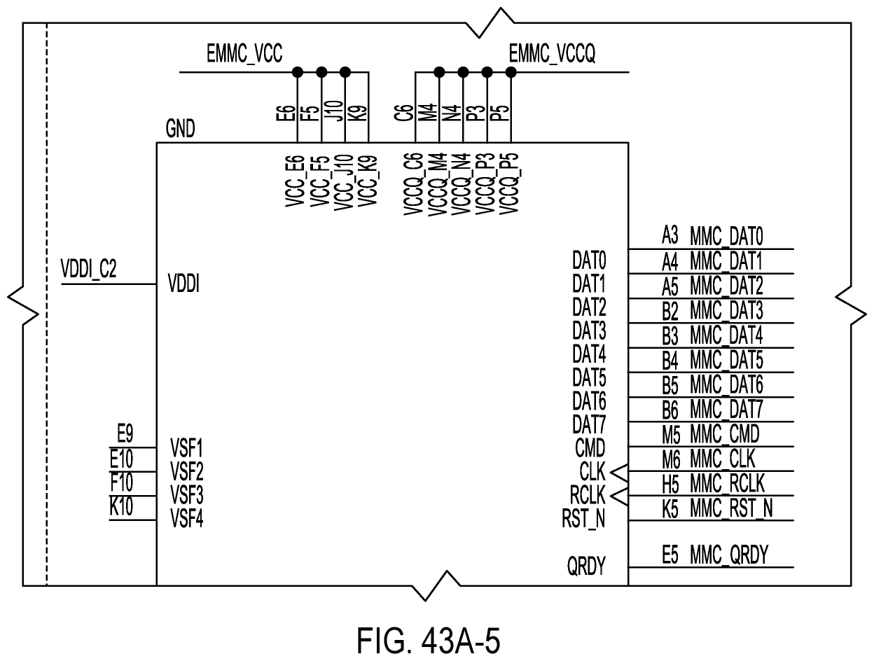

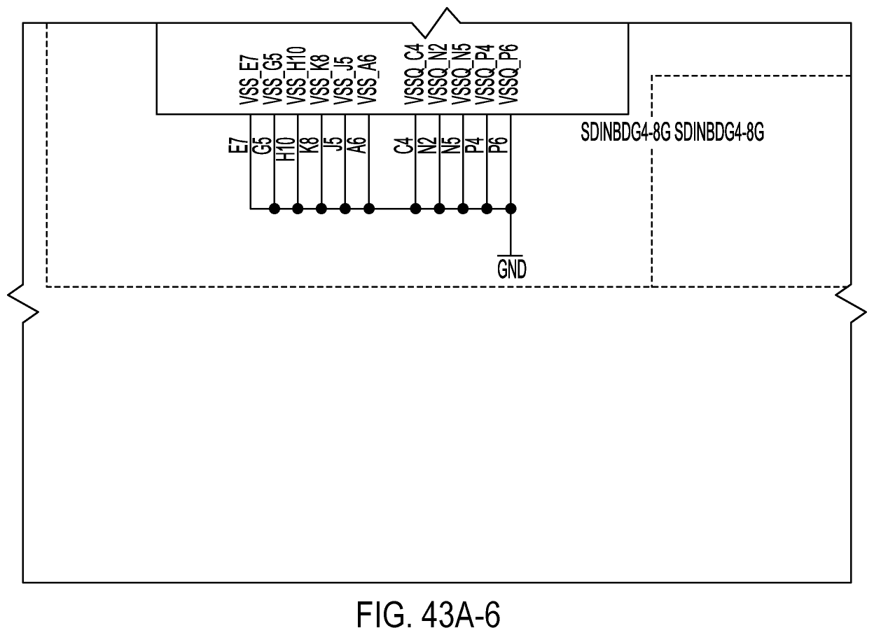

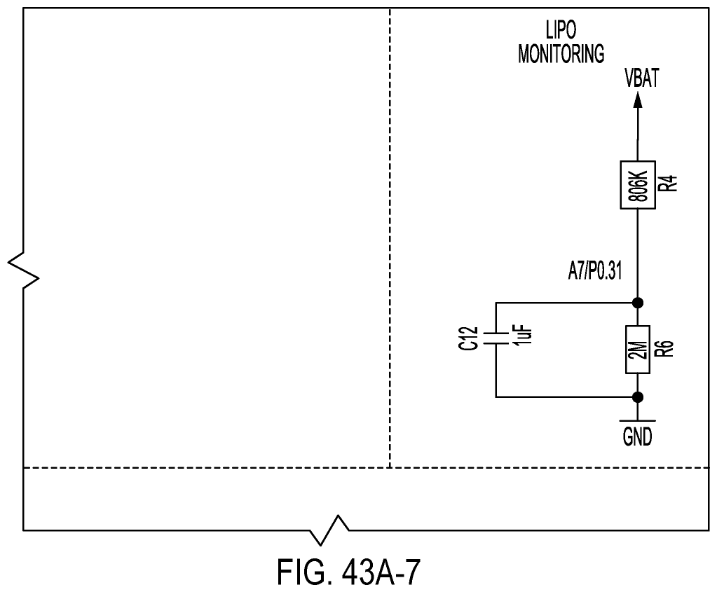

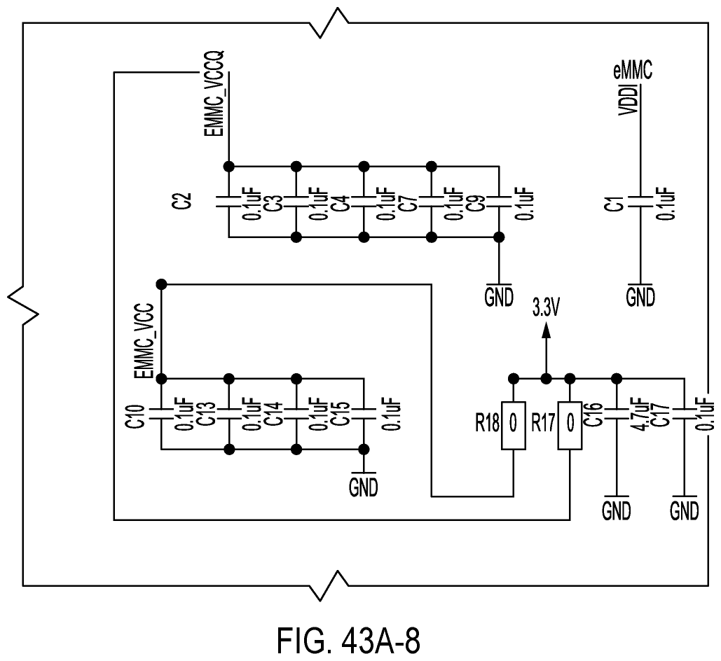

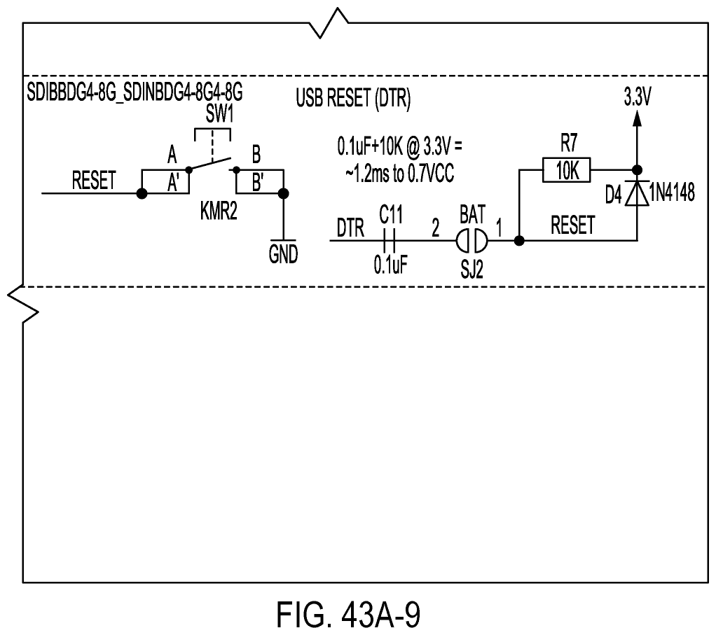

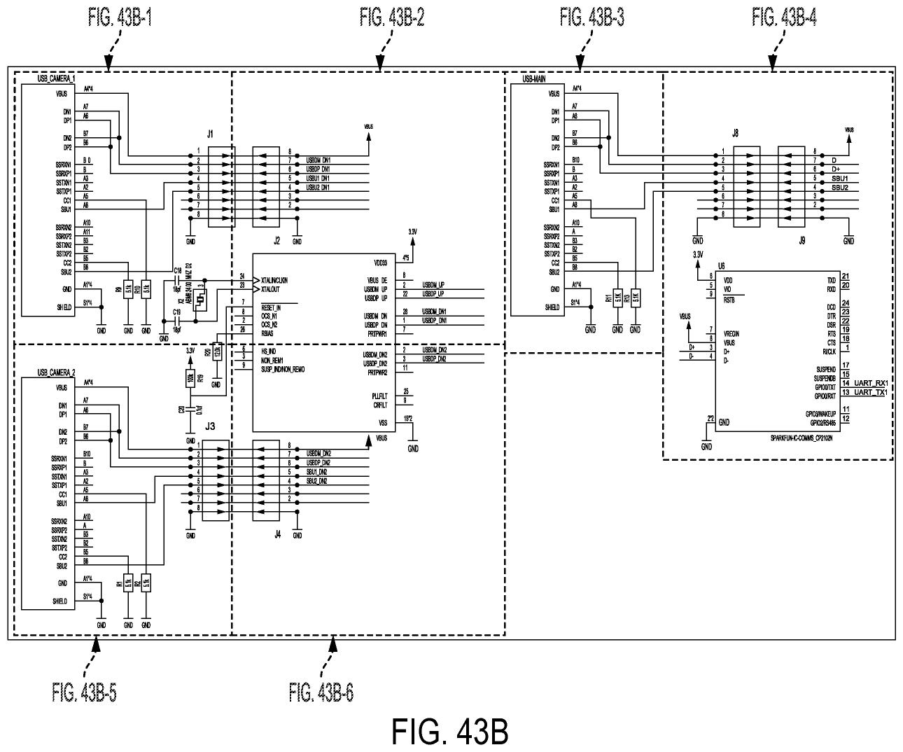

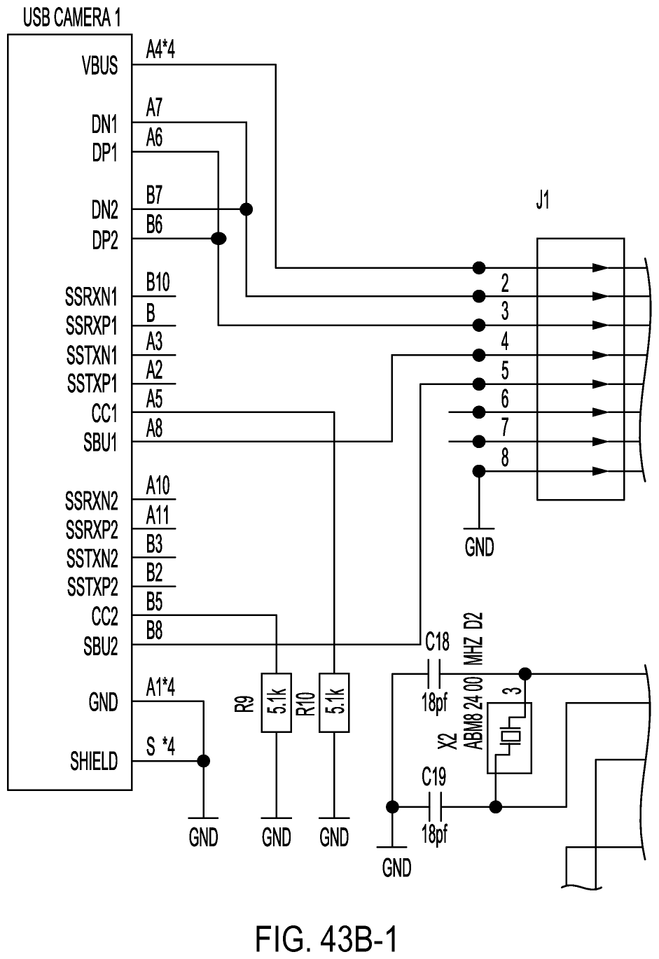

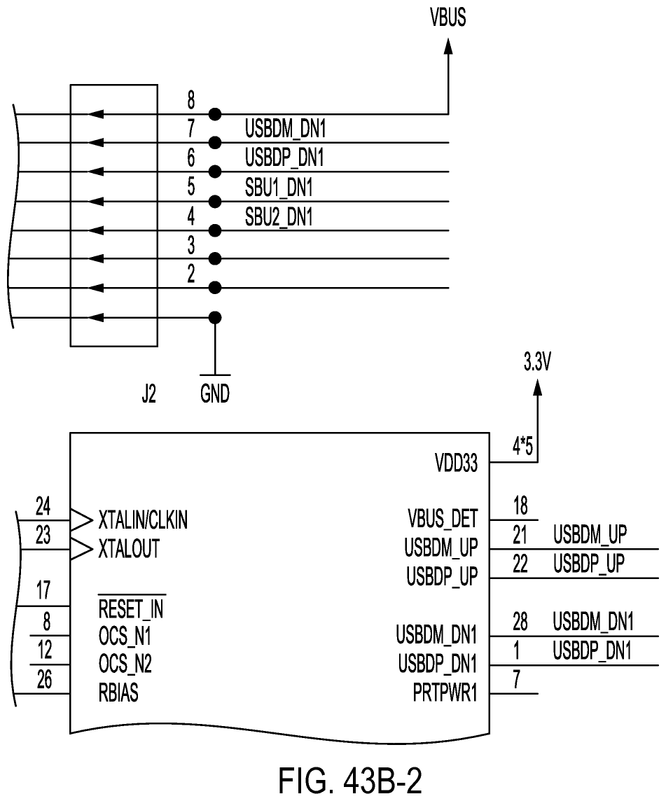

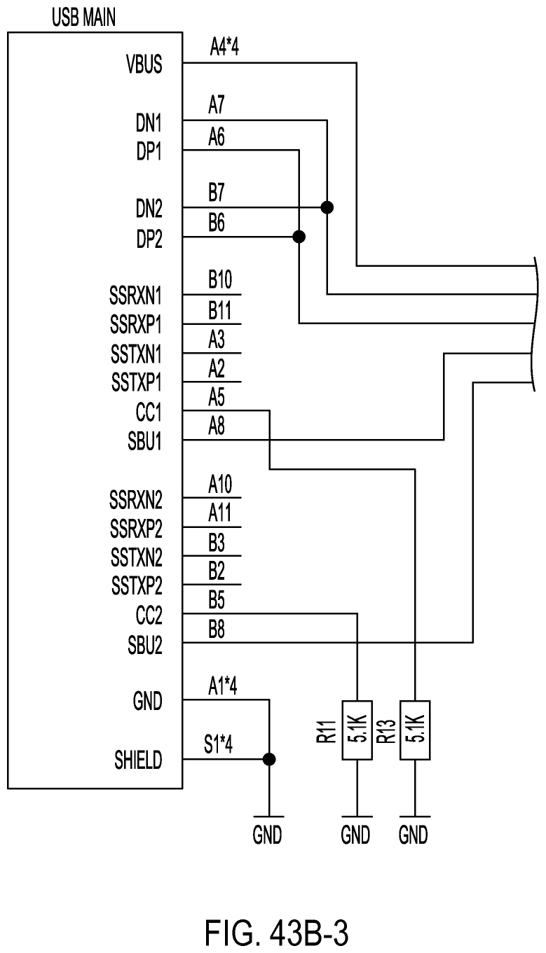

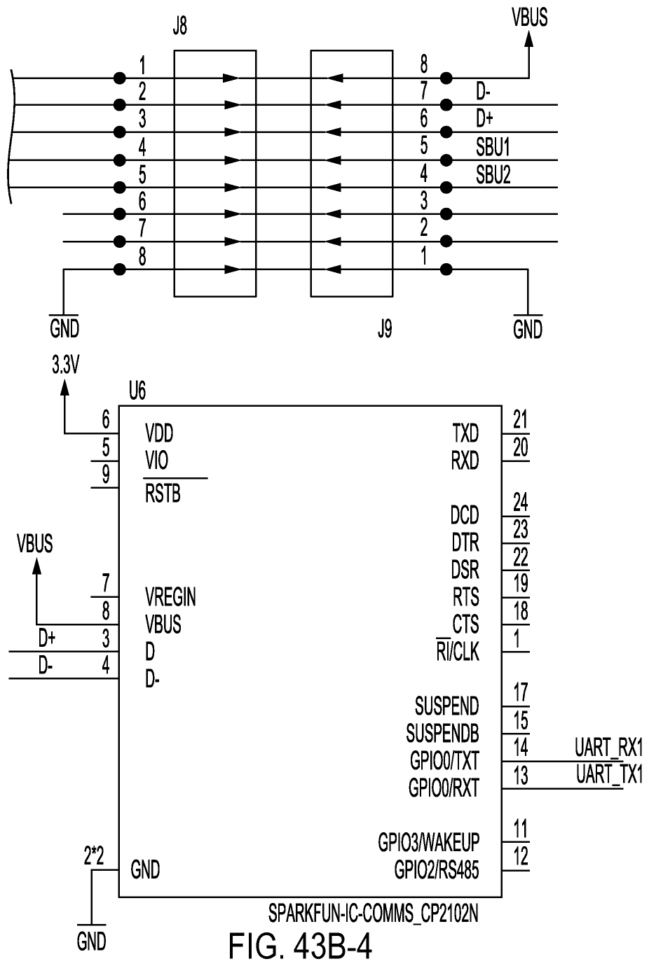

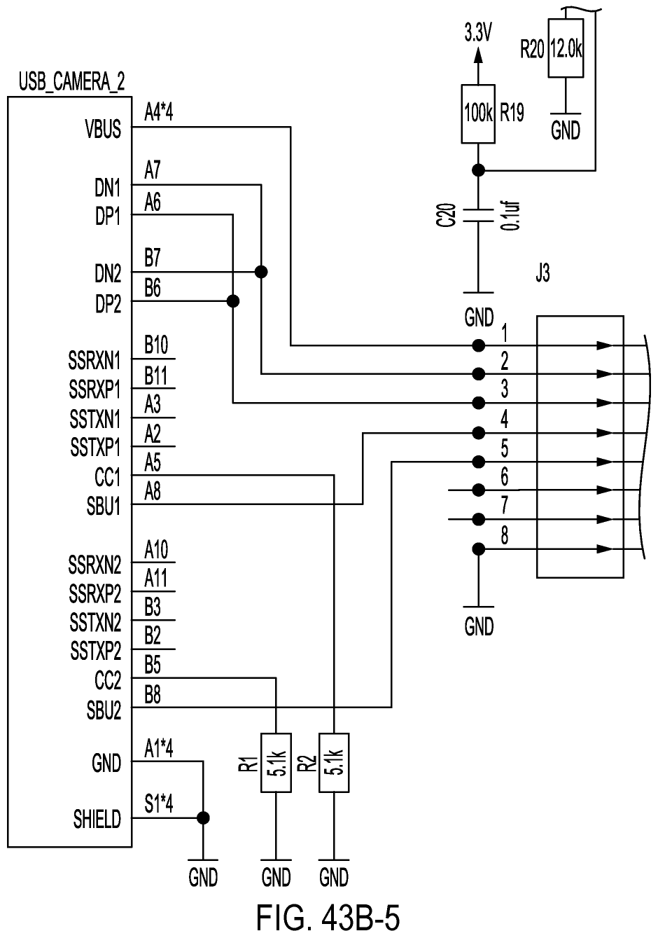

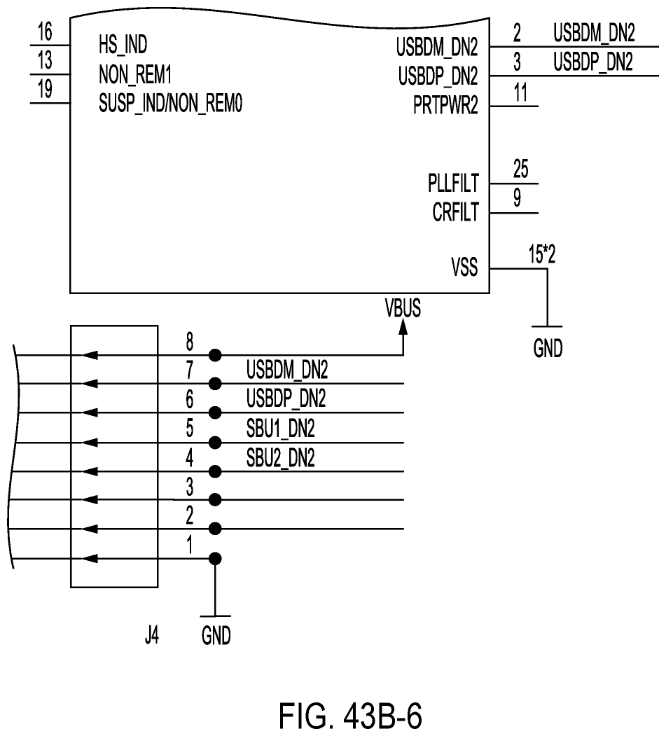

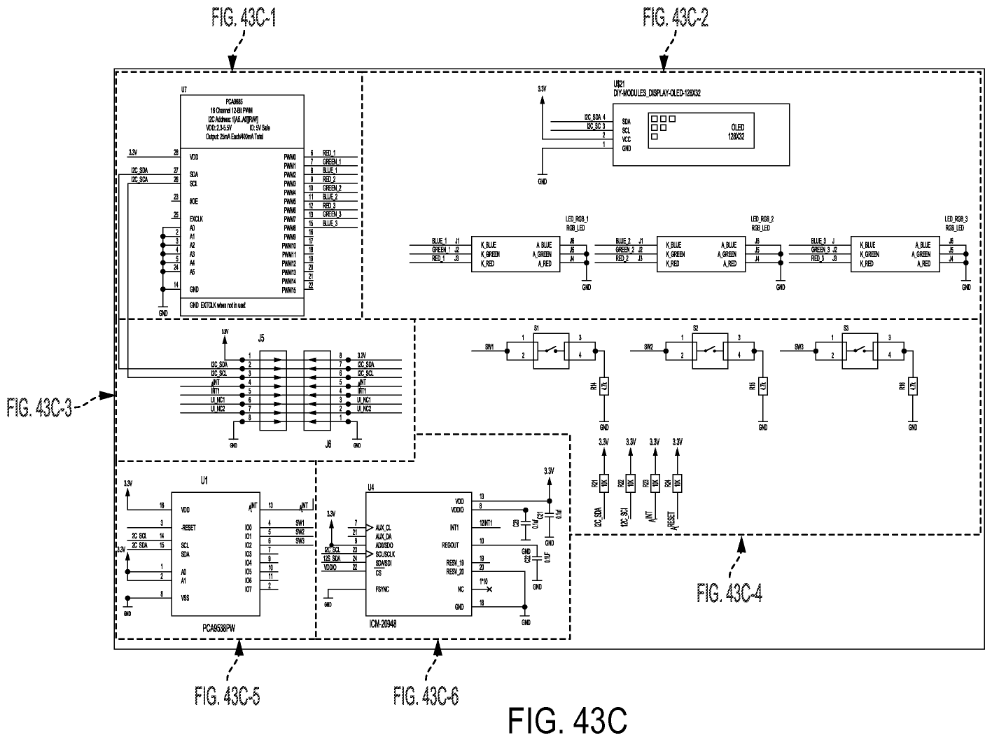

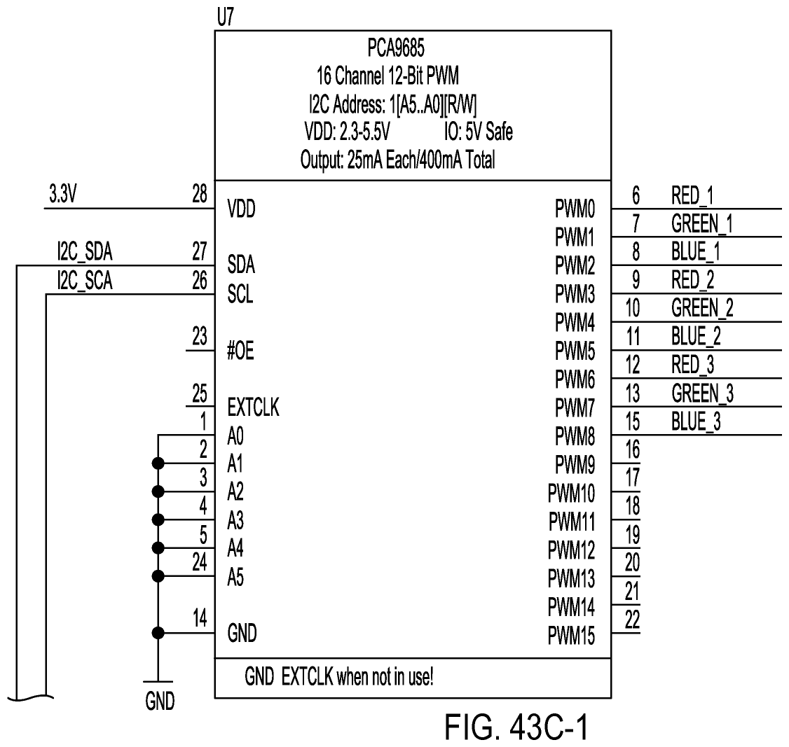

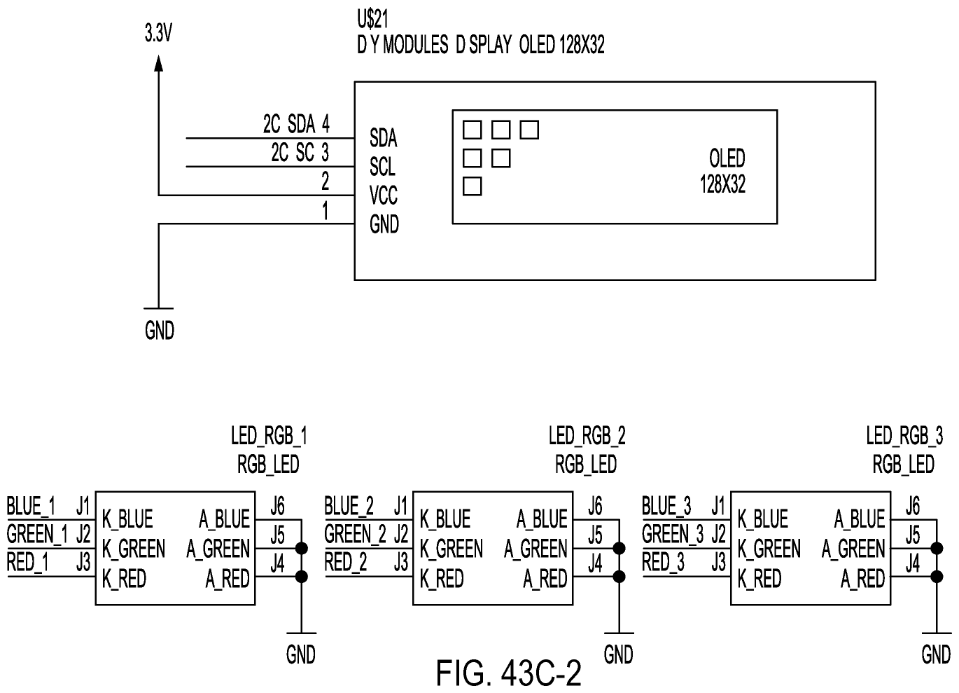

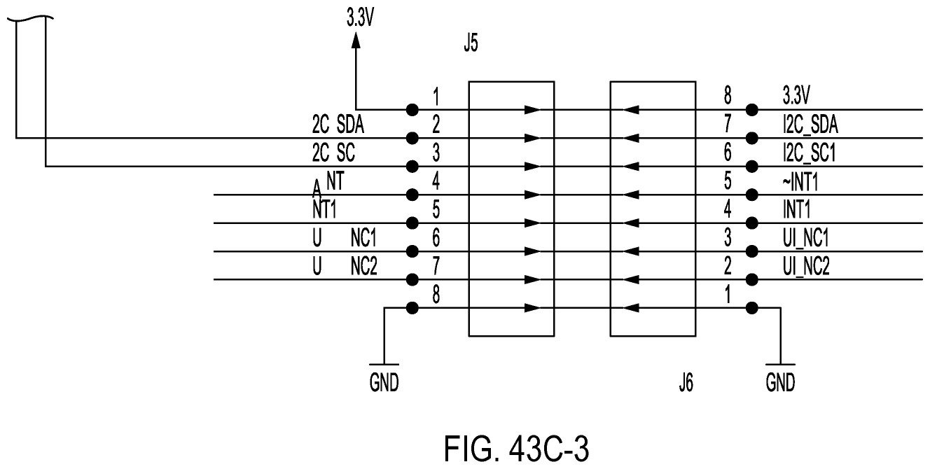

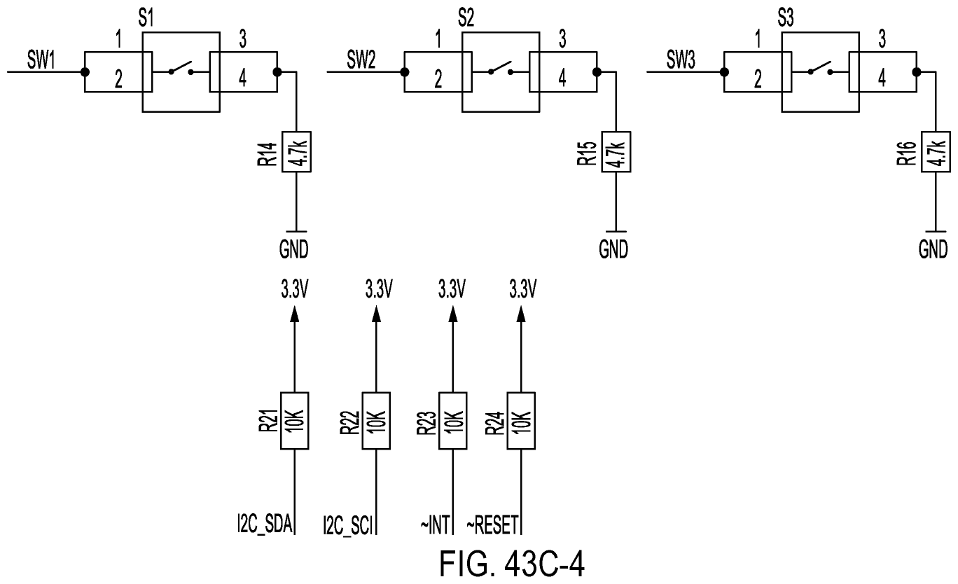

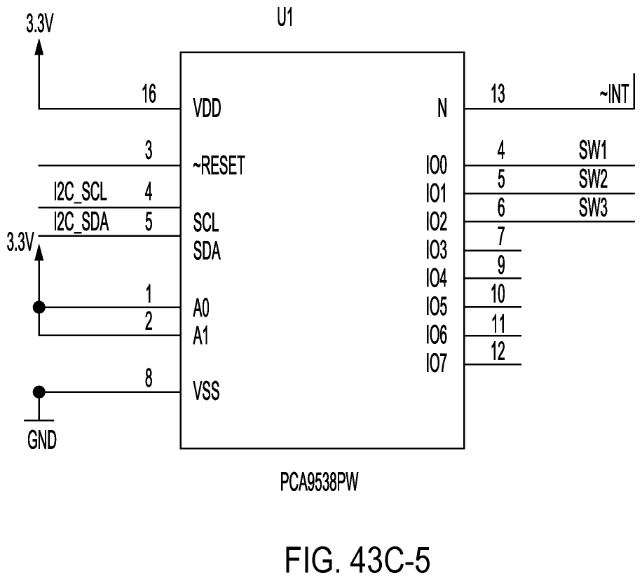

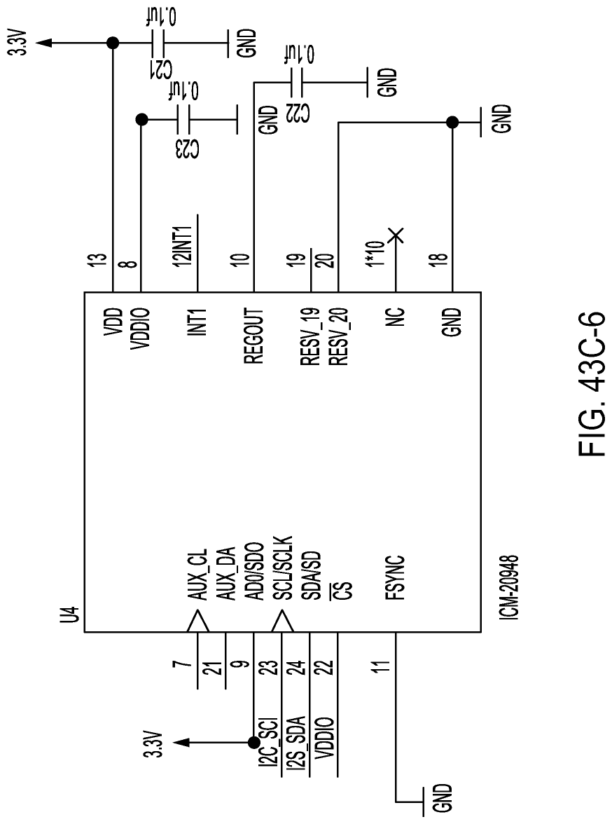

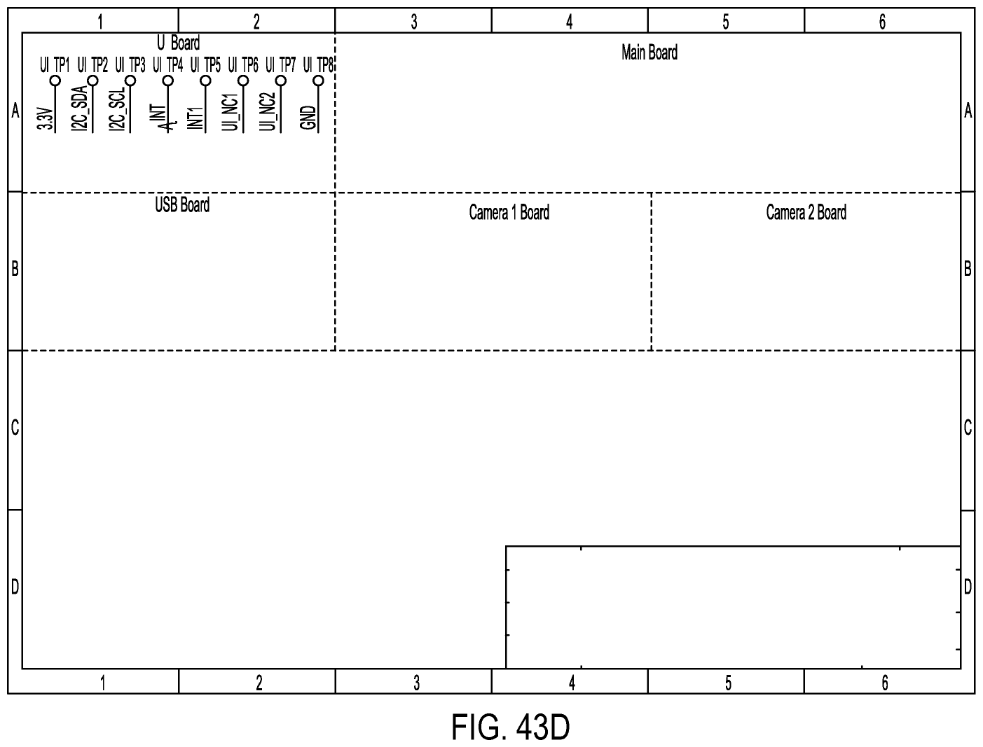

The device 100 can have waterproof wiring, a battery, a printed circuit board (PCB), a wireless transmitter, and connectors. For example, FIGS. 43A-43D illustrate a variation of a PCB schematic for the device 100 having the arrangement of features shown. USB 2.0 can be used which uses four wire conductors regardless of connector type. In a USB-C connector the four wires can be split in parallel to both the top and bottom of the connector (mirrored) so that the user can place the connector in either top or bottom orientation and it will work. USB 3.0 SuperSpeed can be used which usually is packaged as a USB-C device, uses 10+ ground (GND) (11).

We have innovated by being able to connect and control the LEDs and any other sensor to the central processing unit (CPU) by repurposing the USB 3.0 USB-C(SuperSpeed) conductors. This has made our camera USB wiring unique to the Grumpy reusable handle (e.g., handle 102) or module piece (e.g., the module 174). This setup can allow the camera module (e.g., camera 114) to be specifically detected and can prevent non Grumpy cameras (e.g., cameras other than camera 114) being used with the platform e.g., through the detection of the Grumpy camera (e.g., camera 114) uniquely as well possibly based on the SuperSpeed line usage and nothing more mechanically. With our additional unique connectors we have ensured a design with a focus on patient safety.

The device 100 can have reusable components and disposable components. For example, the handle 102 can be reusable, the connector 104 can be disposable, the body 106 can be disposable, the tip 108 can be disposable, the torque transmitter 110 can be disposable, the camera 114 can be disposable, the module 174 can be reusable, or any combination thereof. As another example, the handle 102 can be disposable, the connector 104 can be disposable, the body 106 can be disposable, the tip 108 can be disposable, the torque transmitter 110 can be disposable, the camera 114 can be disposable, the module 174 can be reusable, or any combination thereof.

The lumens 107 of the device 100 can allow for the selective interchange of tools and accessory devices. The tools can pass down the one or more of the lumens 107 (e.g., the central lumen 107c, the side lumens 107s). Larger outer diameter tools with an inner channel that permit the device 100 to be passed within can be used. For example, the device 100 can be used as a guidewire for tools larger than the device 100. Different tools and accessory devices and sheaths mid-procedure can be used as circumstances arise, for example, additional catheter channel/retractable stabilization member can be achieved with the modularity of the device 100.

The modularity of the device 100 can allow for a step-by-step dilation process. For example, a progressive increase in diameter size can be accomplished by inserting progressively larger instruments over each other, for example, by first passing the device 100 over the guidewire, and then passing a larger outer diameter device over the device 100. If a larger incision or hole has been made by the physician, sequential dilation may not necessary, but the device 100 advantageously allows the physician to have flexibility in the approach.

The device 100 can be modularly assembled in-situ.

All patient contact components can be sterile.

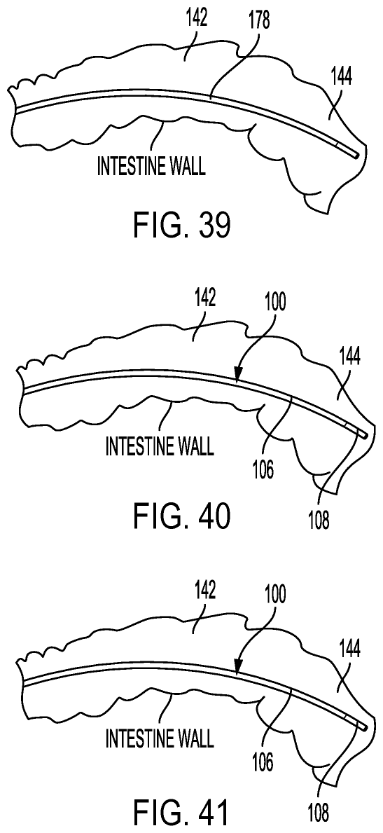

FIGS. 39-41 illustrate an exemplary deployment process. FIG. 39 illustrates that a guidewire 178 can be advanced to a target site 144 in a body cavity 142. FIG. 40 illustrates that the device 100 can be advanced to the target site 144 in the body cavity 142 over the guidewire 178 and that the guidewire 178 can be withdrawn from the device 100. FIG. 41 illustrates that once the guidewire 178 is withdrawn from the device 100, the torque transmitter 110 can be inserted into the device 100.

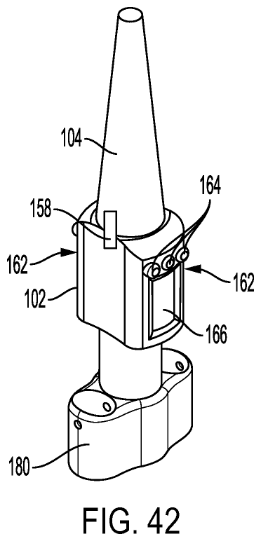

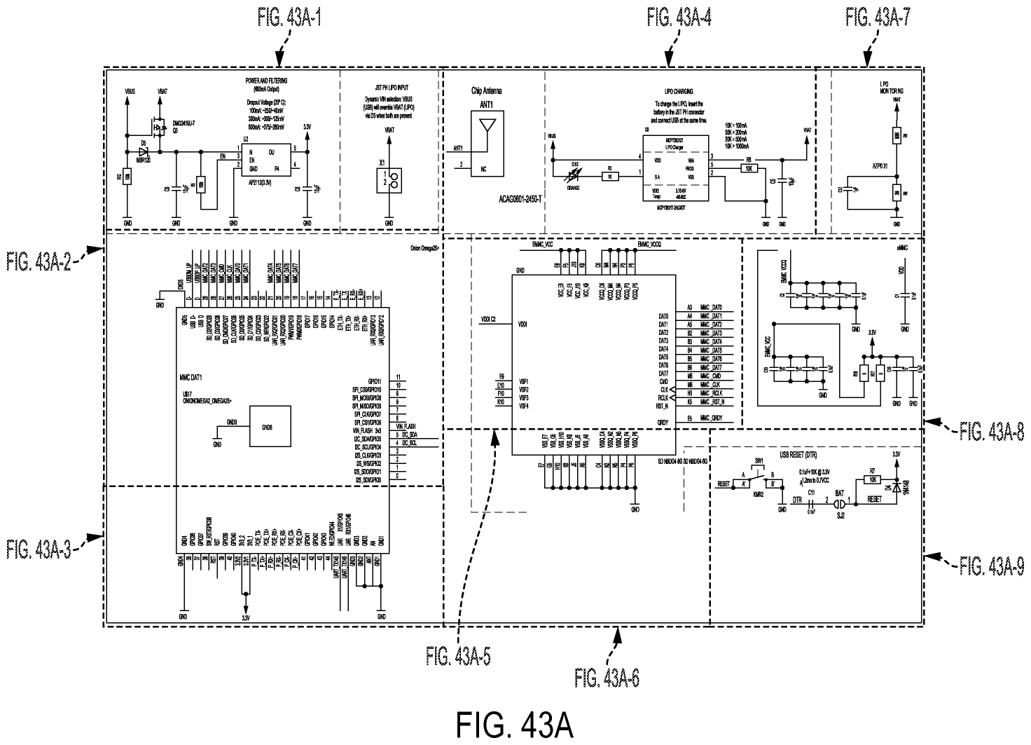

The device 100 can have a removable module (e.g., base) of electronic components. For example, FIG. 42 illustrates that the handle 102 can have a removably connectable module 180 (also referred to as the base 180) of electronic components. The base 180 can have, for example, a battery and other electronics. The base 180 can be removably attached to the handle 102, for example, with a snap fit, a screw fit, a magnetic fit, a friction fit, or any combination thereof. As another example, the module 174 in FIGS. 37 and 38 can be the removable module of electronic components. For example, the module 174 can be the module 180 or the module 174.

The device 100 can be used in the interventional radiological field (e.g., for selective angiography, embolization, and controlled infusion of chemotherapeutic agents is described).

The device 100 can be used to examine and treat various diseases and medical conditions, including, for example, intestinal obstruction treatment.

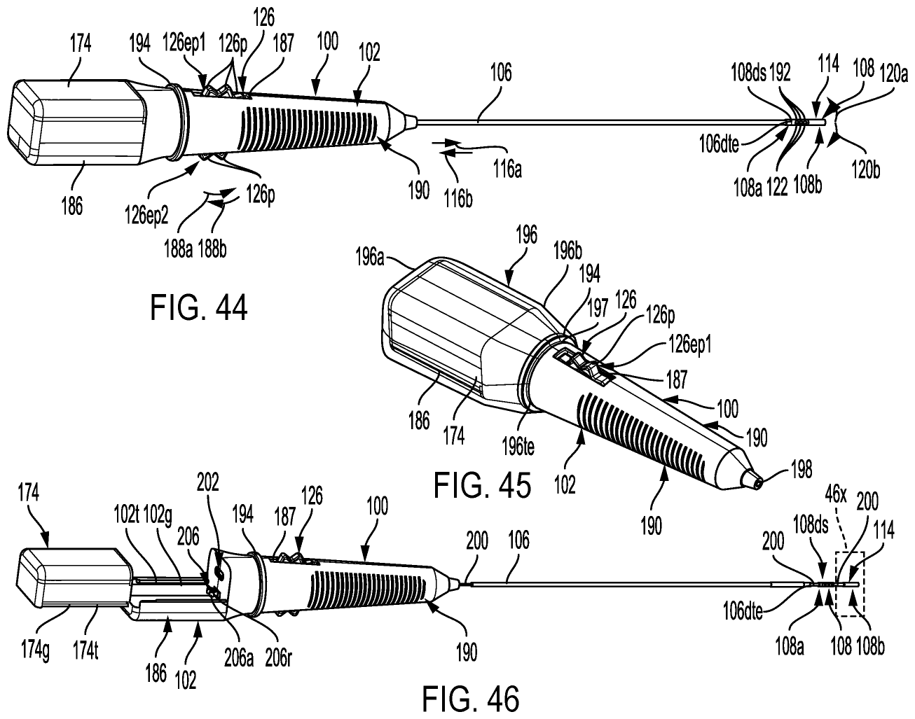

FIG. 44 illustrates that the device 100 can have the arrangement of features shown when the device 100 is in a fully assembled configuration, including, for example, the arrangement of the handle 102, the body 106, the tip 108, the camera 114, and the module 174. FIG. 44 illustrates a straight configuration that the body 106 and the tip 108 can have. FIG. 44 illustrates the device 100 with the body 106 and the tip 108 in a straight configuration.

FIG. 44 illustrates that the module 174 can be removably connected to the device 100, for example, to the handle 102. The module 174 can be in wired and/or wireless communication with the camera 114. The module 174 can have an energy source (e.g., battery) for the camera 114 and/or for electronics on the module 174 (e.g., lights and/or displays on the module 174). The handle 102 can have an extension 186. The module 174 can be removably engageable with the extension 186. For example, FIG. 44 illustrates that the module 174 can be removably engaged with the extension 186.

FIG. 44 illustrates that the handle 102 can have a control 126, for example, one control 126. FIG. 44 illustrates that the control 126 can be, for example, a rotatable wheel. The control 126 can have an ergonomic design to assist with gripping. FIG. 44 illustrates, for example, that the control 126 can have undulations along the perimeter such that the control 126 can have a star-shaped or gear-shaped design with peaks 126p along the perimeter. As another example, the control 126 can have a disc shape with a smooth perimeter, for example, without any undulations or peaks 126p along the perimeter.

FIG. 44 illustrates that the control 126 can extend from the handle 126, for example, from a handle first side 102a and/or from a handle second side 102b such that the control 126 can have a first exposed portion 126ep1 and/or a second exposed portion 126ep2. FIG. 44 illustrates, for example, that the control 126 can extend through openings 187 in the handle 102. The openings 187 can be, for example, slots (e.g., oppositely facing slots) in the handle 102. FIG. 44 illustrates that the handle first side 102a can be opposite the handle second side 102b, and that the first exposed portion 126ep1 can be opposite the second exposed portion 126ep2. Exposing two portions (e.g., opposite portions) of the control 126 can, for example, help users operate the device 100 with one or two hands as the handle 102 is translated and/or rotated during use. FIG. 44 illustrates that the non-exposed portions of the control 126 can be inside the handle 102. Having the non-exposed portions inside the handle 102 can help inhibit or prevent the user from inadvertently articulating the control 126 during use and/or can help inhibit or prevent the control 126 from getting snagged on a user's clothing or on other equipment or tools during use. The handle 102 can thereby function as a cover or guard for the control 126 to inhibit or prevent inadvertent rotation of the control 126. The amount by which the control 126 is exposed outside of the handle 102, the number of peaks 126p exposed, the spacing between the peaks 126p, or any combination thereof can be optimized to assist users to be able to use the handle 102 without looking at the handle 102.

FIG. 44 illustrates that the control 126 can be rotated in a first direction 188a and in a second direction 188b. The first direction 188a can be opposite the second direction 188b. For example, the first direction 188a can be counterclockwise rotation of the control 126 and the second direction 188b can be clockwise rotation of the control 126 or vice versa. FIG. 44 illustrates, for example, that when the control 126 is rotated in the first direction 188a, the tip 108 can move in the first direction 120a to form various first arcs, and that when the control 126 is rotated in the second direction 188b, the tip 108 can move in the second direction 120b to form various first arcs.

FIG. 44 illustrates that that the handle 102 can have a surface 190 having ridges and/or grooves to assist a user with gripping the handle 102. The side of the handle 102 opposite the side shown in FIG. 44 can also have the surface 190.

FIG. 44 illustrates that the tip 108 can include a deflectable section 108ds and the camera 114. FIG. 44 illustrates that the camera 114 can extend distally away from the deflectable section 108ds. FIG. 44 illustrates that the deflectable section 108ds can be between the body 106 and the camera 114. FIG. 44 illustrates, for example, that the tip 108 can have a tip first end 108a and a tip second end 108b. The tip first end 108a can be proximal the tip second end 108b or vice versa. For example, the tip first end 108a can be a proximal end of the tip 108 and the tip second end 108b can be a distal end of the tip 108 or vice versa. The tip first end 108a can have the deflectable section 108ds and the tip second end 108b can have the camera 114. For example, FIG. 44 illustrates that the tip first end 108a can be the deflectable section 108ds and that the tip second end 108b can be the camera 114. As another example, the positions of the deflectable section 108ds and the camera 114 in FIG. 44 can be swapped with each other such the tip first end 108a can be the camera 114 and the tip second end 108b can be the deflectable section 108ds. As another example, the deflectable section 108ds can be a distal end of the body 106.

FIG. 44 illustrates that the deflectable section 108ds can have notches 122 and hinges 192. FIG. 44 illustrates, for example, that the hinges 192 can be at the base of the notches 122. The tip 108 can flex about the hinges 192, for example, as the control 126 is rotated (e.g., in the first direction 188a or in the second direction 188b). The notches 122 and the hinges 192 can allow the tip 108 to flex when the actuators 112 are tensioned (e.g., pulled in direction 116b). The notches 122 and the hinges 192 can thereby assist in articulation of the tip 108.

FIG. 44 illustrates that the body 106 and the tip 108 can be removably attached to each other. FIG. 44 illustrates that the body 106 can extend into the tip 108. FIG. 44 illustrates, for example, that a body distal terminal end 106dte can be inside the tip 108 (e.g., inside the tip first end 108a). For example, FIG. 44 illustrates that the body distal terminal end 106dte can be inside the deflectable section 108ds. The tip 108 can be removably attachable to the distal end of the body 106, for example, with a snap fit, a clip fit, a screw fit, a friction fit, or any combination thereof. For example, FIG. 44 illustrates that the proximal end of the tip 108 can extend over the distal end of the body 106 with a friction fit. The tip 108 can be modular such that various tips 108 (e.g., other tips 108 that are exactly the same as the tip 108 shown in FIG. 44 or which have different features, such as more or less cameras 114, more or less notches 122, more or less hinges 192) can be removably attached to the body 106. As another example, the tip 108 can be fixedly attached to the body 106, for example, with glue. In such cases, the tip 108 may not be removably attached to the body 106, in which case the body 106 and the tip 108 can be a single subassembly that can be removably attachable to the handle 102.

FIG. 44 illustrates that the handle 102 can have a ridge 194. The ridge 194 can be a barrier between the portion of the device 100 distal the ridge 194 and the portion of the device 100 proximal the ridge 194, for example, to help keep the module 174 sterile during use. The ridge 194 can extend partially or completely around the handle 102. For example, FIG. 44 illustrates that the ridge 194 can extend completely around the handle 102. A cover 196 (e.g., see FIG. 45) can be removably attached to the handle 102, for example, around the module 174 and around the extension 186, for example, to help keep the module 174 sterile during use. The opening of the cover 196 can be adjacent the ridge 194, for example, proximal the ridge 194 or distal the ridge 194. The distal terminal end of the cover 196 can be adjacent the ridge 194, for example, proximal the ridge 194 or distal the ridge 194. The cover 196 can permit the use of the module 174 in sterile environments so that the module 174 can be reused. The cover 196 can be, for example, a sterile cover.

FIG. 45 illustrates that a cover 196 can be removably attached to the handle 102. FIG. 45 illustrates that the cover 196 can have a cover first end 196a and a cover second end 196b. The cover first end 196a can be a closed end of the cover 196 and the cover second end 196b can be an open end of the cover 196. For example, FIG. 45 illustrates that the cover second end 196b can have a cover terminal end 196te and an opening 197. The cover terminal end 196te can, for example, define the opening 197. The cover first end 196a can be the proximal end of the cover 196 and the cover second end 196b can be the distal end of the cover 196 or vice versa. For example, the cover terminal end 196te can be the distal terminal end of the cover 196.

When the cover 196 is removably attached to the handle 102, the module 174 and/or the handle 102 can be inside the cover 196. For example, FIG. 45 illustrates that when the cover 196 is removably attached to the handle 102, the module 174 and the extension 186 can be inside (e.g., completely inside) the cover 106.

When the cover 196 is removably attached to the handle 102, the cover terminal end 196te can be proximal or distal the ridge 194. For example, FIG. 45 illustrates that when the cover 196 is removably attached to the handle 102, the cover terminal end 196te can be distal the ridge 194.

The cover 196 can be flexible or rigid. The cover 196 can be elastic and/or can have an elastic band integrated in the cover 196 (e.g., in the cover terminal end 196te) such that the cover 196 can be stretched over the ridge 194 and form a seal against the handle 102, for example, distal the ridge 194. As another example, a connector (e.g., one or more rubber bands) can be placed over the cover 196 distal and/or proximal the ridge 194 to releasably secure the cover 196 in the position shown in FIG. 45 to form a seal against the handle 102 so that the module 174 can be kept sterile during use. As yet another example, a connector (e.g., a string) can be tied around the cover 196 distal and/or proximal the ridge 194 to releasably secure the cover 196 in the position shown in FIG. 45 to form a seal against the handle 102 so that the module 174 can be kept sterile during use. The cover 196 can be, for example, a cap that can be removably attachable to the handle 102.

FIG. 45 illustrates that the cover 196 can, for example, permit the use of the module 174 in sterile environments so that the module 174 can be reused with multiple devices 100, for example, with a first device 100 during a first procedure and with a second device 100 during a second procedure.

FIG. 45 illustrates that the cover 196 can be transparent. For variations in which the module 174 has controls, lights, and/or displays, this can allow the user to see the controls, lights, and/or displays on the module 174 during use.

FIG. 45 illustrates that the handle 102 can have a channel 198. The handle 102 and the body 106 can be releasably attached to each other, for example, by inserting the body 106 in the channel 198. The proximal end of the body 106 can be removably attachable to the distal end of the handle 102, for example, with a snap fit, a clip fit, a screw fit, a friction fit, or any combination thereof. For example, FIGS. 44 and 45 illustrate that the proximal end of the body 106 can extend into the channel 198 at the distal end of the handle 102 with a friction fit. As another example, the body 106 can be fixedly attached to the handle 102, for example, with glue. In such cases, the body 106 may not be removably attached to the handle 102, in which case the handle 102 and the body 106 can be a single subassembly that can be removably attachable to the tip 108 and/or disposable.

FIG. 45 illustrates that the handle 102 can have two surfaces 190, for example, on opposite sides of the handle 102.

FIG. 46 illustrates that the device 100 in FIG. 45 can be modular, with FIG. 45 illustrating the modular components in a fully assembled configuration. For example, FIG. 46 illustrates that the body 106 can be removably attachable to the handle 102, that the module 174 can be removably attachable to the handle 102, that the tip 108 can be removably attachable to the body 106, that the camera 114 can be removably attachable to the deflectable section 108ds, or any combination thereof. These components can be removably connectable, for example, with snap fits, clip fits, screw fits, friction fits, or any combination thereof. The actuators 112 are shown transparent in FIG. 46 for illustrative purposes.

FIG. 46 illustrates that the device 100 can have a connector 200 and a connector 202. The connector 200 can connect the camera 114 to the handle 102, for example, to the connector 202. The connector 200 can be an electrical connector. The connector 200 can be, for example, a wire that electrically connects the camera 114 to the connector 202. For example, FIG. 46 illustrates that a proximal end of the connector 200 can be connected to the camera 114 and that a distal end of the connector 200 can be connected to the connector 202. FIG. 46 illustrates that the connector 200 can extend through the deflectable section 108ds, through the body 106, and through the handle 102, for example, through one or more lumens (e.g., lumens 107) in the device 100. The connector 200 can be the torque transmitter 110. The connector 200 can function as a torque transmitter (e.g., the torque transmitter 110). As another example, the connector 200 may not be a torque transmitter 110.

FIG. 46 illustrates that the connector 202 can be an electrical connector. For example, FIG. 46 illustrates that the connector 202 can releasably interface with a connector 204 on the module 174 (e.g., see FIG. 48) to electrically connect the camera 114 to the module 174. The connector 202 can be, for example, a pogo pin connector. The connector 202 can be anywhere on the device 100. For example, FIG. 46 illustrates that the connector 202 can be on the handle 102 in the position shown, for example, proximal the ridge 174. When the module 174 is releasably attached to the handle 102, for example, as shown in FIG. 44, the connector 202 and the connector 204 can be releasably engaged with each other such that the connector 200 can electrically connect the camera 114 to the module 174. When the connector 202 and the connector 204 can be releasably engaged with each other, data and/or power can be transmitted to the camera 114 from the module 174. When the connector 202 and the connector 204 can be releasably engaged with each other, data can be transmitted to the module 174 from the camera 114. FIG. 46 illustrates, for example, that the camera 114 can be in wired communication with the module 174, for example, via the connector 200 and that the module 174 can have a battery for the camera 114. As another example, the camera 114 can be in wireless communication with the module 174 such that data can be wirelessly transmitted between the camera 114 and the module 174 via a wireless data communication link. As another example, the camera 114 can be in wired and wireless communication with the module 174. As still yet another example, the camera 114 may not be in communication with the module 174.

FIG. 46 illustrates that the device 100 can have a connector 206. The connector 206 can be, for example, a clip. The clip can be snap. The clip can have a snap fit. The connector 206 can have a first portion 206a and a second portion 206b (e.g., see FIG. 48). The first portion 206a can be releasably engageable with the second portion 206b (e.g., see FIG. 48). The first portion 206a can be on the handle 102 and the second portion 206b can be on the module 174 or vice versa. FIG. 46 illustrates that the first portion 206a can be a female portion and that the second portion 206b can be the male portion. FIG. 46 illustrates, for example, that the second portion 206a can have a recess 206r that can releasably receive the second portion 206b. The handle 102 and the module 174 can be releasably attached to each other via the connector 206. For example, FIG. 44 illustrates that when the module 174 is releasably attached to the handle 102, the first portion 206a can be releasably engaged with the second portion 206b.

FIG. 46 illustrates that the handle 102 (e.g., the extension 186) can have tongues 102t and grooves 102g and that the module 174 can have tongues 174t and grooves 174g. Each one of the tongues 102t can slide in the one of the grooves 174g, and each of the tongues 174t can slide in one of the grooves 102g. FIG. 46 illustrates that the slidable engagement between the tongues 102t and the grooves 174g and/or between the tongues 174t and the grooves 102g can align the male female portion 206a with the male portion 206b, can align the connector 204 with the connector 202, or can align both. The tongues 102t are also referred to as first tongues 102t and handle tongues 102t, the grooves 102g are also referred to as first grooves 102g and handle grooves 102g, the tongues 174t are also referred to as second tongues 174t and module tongues 174t, and the grooves 174g are also referred to as second grooves 174g and module grooves 102g. FIG. 46 illustrates that the device 100 can have the first and second tongues 102t, 174t and the first and second grooves 102g, 174g. As another example, the device 100 may only have the first tongues 102t and the second grooves 174g (e.g., the device 100 may not have the second tongues 174t and the first grooves 102g). As another example, the device 100 may only have the second tongues 174t and the first grooves 102g (e.g., the device 100 may not have the first tongues 102t and the second grooves 174g).

FIGS. 44-46 illustrates that the device 100 may not have the connector 104. FIG. 46 illustrates, for example, that the proximal end of the body 106 can be connected to a distal end of the handle 102.

FIGS. 44-46 illustrate that the module 174 can be reusable, and that the rest of the device 100, including the handle 102, the body 106, the tip 108, the actuators 112, and the cover 196, can be disposable. As another example, the entire device 100, including the module 174, can be disposable.

FIGS. 44-46 illustrate that the handle 102, the body 106, the tip 108, the actuators 112, and the module 174 or any combination thereof can be modular and replaceable before, during, and/or after use. The handle 102, the body 106, the tip 108, the actuators 112, or any combination thereof can be removably attached from the device 100 at any given time, for example, before use, during use, and/or after use. FIGS. 44-46 illustrate that the camera 114 can be removably attachable to the deflectable section 108ds. As another example, the camera 114 can be fixedly attached to the deflectable section 108ds. The camera 114 can have, for example, a field of view from 120 degrees to 170 degrees, including every 1 degree increment within this range (e.g., 120 degrees, 150 degrees, 170 degrees).

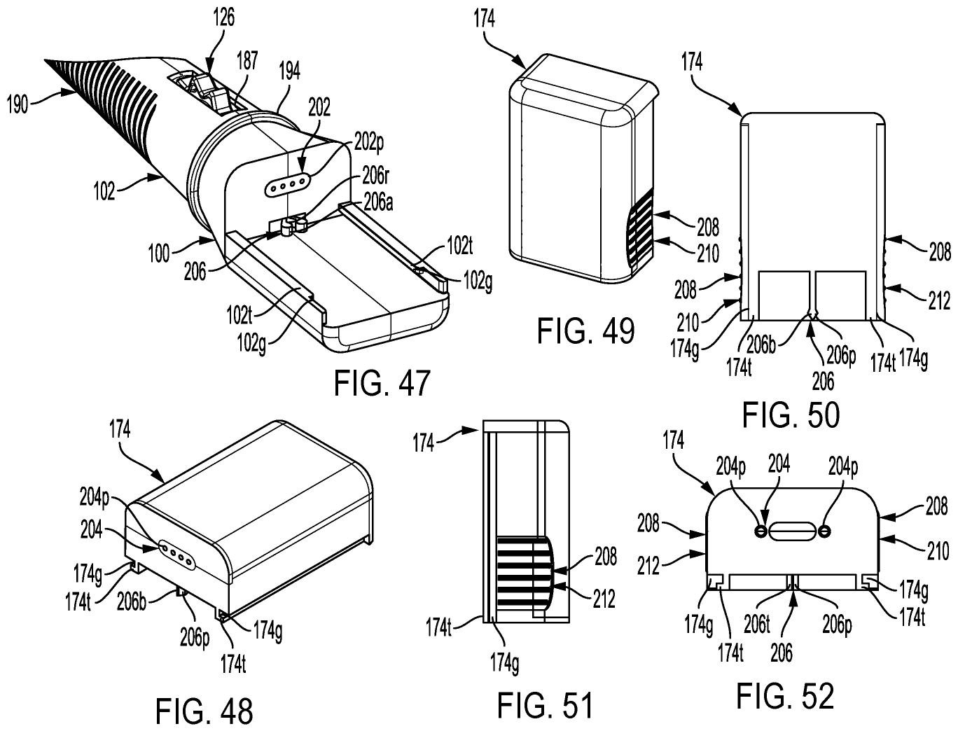

FIGS. 47 and 48 illustrate the module 174 detached from the handle 102. FIG. 47 illustrates that the connector 202 can be, for example, a pogo pin connector having pins 202p. The connector 202 can have, for example, 2 to 12 pins, including every 1 pin increment within this range such as 2 pins, 4 pins, 12 pins). For example, 47 illustrates that the connector 202 can have four pins 202p. The connector 202 can thereby be a four pin pogo pin connector. FIG. 48 illustrates that the connector 204 can be, for example, a pogo pin connector having pins 204p. The connector 204 can have, for example, 2 to 12 pins, including every 1 pin increment within this range such as 2 pins, 4 pins, 12 pins). For example, 48 illustrates that the connector 204 can have four pins 204p. The connector 204 can thereby be a four pin pogo pin connector. FIGS. 47 and 48 illustrate that the connector 202 can have the same number of pins as the connector 204. FIG. 47 illustrates that the device 100 (e.g., the extension 186) can have two tongues 102t and two grooves 102g. FIG. 48 illustrates that the module 174 can have two tongues 174t and two grooves 174g. FIG. 48 illustrates that the second portion 206b of the connector 206 can be between (e.g., halfway between) the two tongues 174t. FIG. 48 illustrates that the second portion 206b of the connector 206 can have a protrusion 206p that can be releasably received in the recess 206r.

FIG. 49 illustrates that that the module 174 can have a surface 208 having ridges and/or grooves to assist a user with gripping the module 174. The side of the module 208 opposite the side shown in FIG. 49 can also have the 208.

FIG. 49 illustrates that the device 100 can have a control 210 that can turn the camera 114 on and off. The control 210 can be on the module 174 or on the handle 102. For example, FIG. 49 illustrates that the control 210 can be on the module 174. As another example, the control 210 can be anywhere on the handle 102. The control 210 can be a button, a switch, a slide, a knob, or any combination thereof. For example, FIG. 49 illustrates that the control 210 can be a pressable button that can be pressed a first time, for example, to turn the camera 114 on and that can be pressed a second time, for example, to turn the camera 114 off. FIG. 49 illustrates that the control 210 can have the surface 208. As another example, the device 100 may not have the control 210. In such cases, the camera 114 can turn on automatically, for example, when the module 174 is attached to the handle 102 (e.g., when the connector 202 in in contact with the connector 204), and the camera 114 can turn off automatically, for example, when the module 174 is detached from the handle 102 (when the connector 202 breaks contact with the connector 204).

FIG. 49 illustrates that the control 210 can have the shape and position shown.

FIG. 50 illustrates that the module 174 can have two surfaces 208, for example, on opposite sides of the module 174.

FIG. 50 illustrates that the device 100 can have a control 212 that can, for example, control the illuminators 138 of the camera 114. The control 212 can be on the module 174 or on the handle 102. For example, FIG. 50 illustrates that the control 212 can be on the module 174. As another example, the control 212 can be anywhere on the handle 102. The control 212 can be a button, a switch, a slide, a knob, or any combination thereof. For example, FIG. 50 illustrates that the control 212 can be a pressable button that can be pressed one or multiple times to turn on the illuminators 138, turn off the illuminators 138, and/or to cycle through one or more settings of the illuminators 138. For example, the control 212 can be a pressable button that can be pressed a first time, for example, to turn on the illuminators 138 of the camera 114 to a first brightness, can be pressed a second time, for example to increase the intensity of the illuminators 138 to a second brightness greater than the first brightness, can be pressed a third time, for example, to change the light color from white light to another color (e.g., to blue and/or to red), can be pressed a four time, for example, to activate a first strobe frequency of the illuminators 138 of the camera 114, can be pressed a fifth time, for example, to activate a second strobe frequency of the illuminators 138 of the camera 114 greater than the first strobe frequency, can be pressed a sixth time, for example, to deactivate the strobe frequency (e.g., the first strobe frequency or the second strobe frequency) of the illuminators 138 of the camera 114, can be pressed a seventh time, for example, to turn off the illuminators 138, or any combination thereof and in any order. FIG. 50 illustrates that the control 212 can have the surface 208. FIG. 50 illustrates that the control 212 can be opposite the control 210. As another example, the control 212 can be next to the control 210. As another example, the device 100 may not have the control 212. In such cases, the illuminators 138 can turn on automatically, for example, when the module 174 is attached to the handle 102 (e.g., when the connector 202 in in contact with the connector 204), and the illuminators 138 can turn off automatically, for example, when the module 174 is detached from the handle 102 (when the connector 202 breaks contact with the connector 204).

FIG. 51 illustrates that the control 212 can have the shape and position shown.

FIG. 52 illustrates that the connector 204 can have two pins 204p. The connector 204 can thereby be a two pin pogo pin connector.

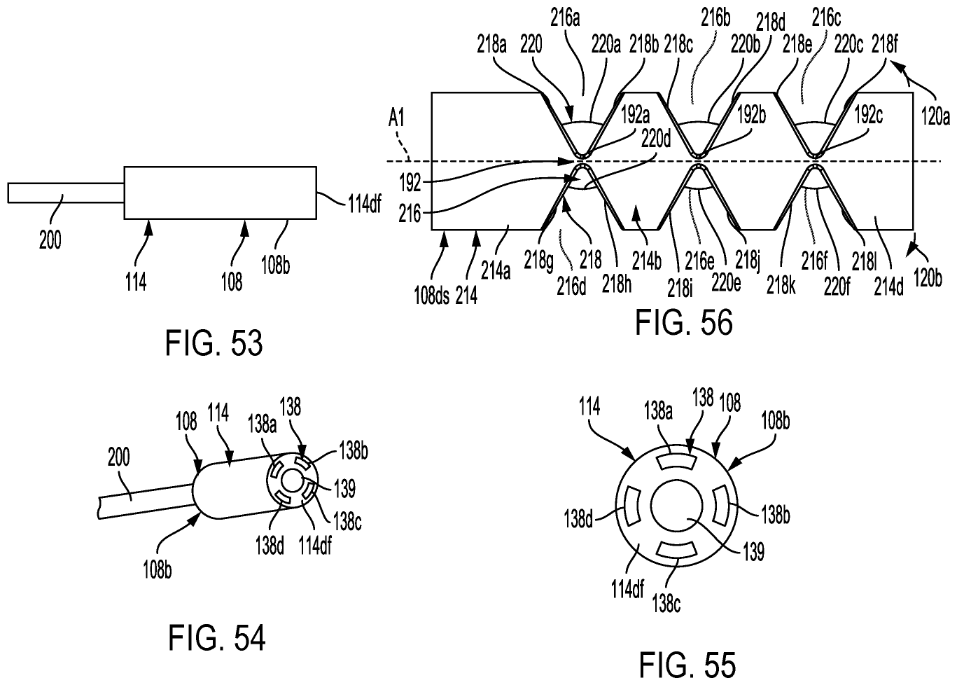

FIG. 53 illustrates a side view of section 46x in FIG. 46 of the camera 114 and the connector 200. FIG. 53 illustrates that the connector 200 can extend from the camera 114, for example, from a proximal end of the camera 114. FIG. 53 illustrates that the distal end of the connector 200 can be connected to the camera 114. FIG. 53 illustrates that the illuminators 138 and the camera lens 139 (e.g., see FIGS. 54 and 55) may not protrude from the distal face 114df of the camera 114. This can reduce the profile of the camera 114 and thereby lessen the likelihood of the illuminators 138 and/or the camera lens 139 from contacting or disturbing tissue as the tip 108 (e.g., the deflectable section 108ds) is articulated. When the device 100 is in a straight configuration, the distal face 114df, the illuminators 138, and/or the camera lens 139 can be distal terminal end of the device 100. For example, FIG. 53 illustrates that when the device 100 is in a straight configuration, the distal face 114df of the camera 114 can be the distal terminal end of the device 100.

FIG. 54 illustrates a front perspective view of section 46x in FIG. 46 of the camera 114 and the connector 200. FIG. 54 illustrates that the device 100 can have illuminators 138. The illuminators 138 can be anywhere on the tip 108. For example, the illuminators 138 can be on the deflectable section 108ds and/or on the camera 114. For example, FIG. 54 illustrates that the illuminators 138 can be on the camera 114. FIG. 54 illustrates that the illuminators 138 can be on a distal end of the camera 114, for example, on a distal face 114df of the camera 114. The device 100 can have, for example, 1-10 illuminators 138, including every 1 illuminator 138 increment within this range (e.g., 1 illuminator, 2 illuminators, 4 illuminators, 10 illuminators). For example, FIG. 54 illustrates that the camera 114 can have 4 illuminators 138, for example, positioned around a lens 139 (also referred to as the camera lens 139) of the camera 114. FIG. 54 illustrates that the illuminators 138 can have a crescent shape. FIG. 54 illustrates that the center of the illuminators 138 can be spaced 90 degrees apart from each other around the camera lens 139.

FIG. 54 illustrates that the illuminators 138 can include a first illuminator 138a, a second illuminator 138b, a third illuminator 138c, and a fourth illuminator 138d. The illuminators 138 can emit any wavelength of light. The illuminators 138 can, for example, emit visible light, near infrared light, infrared light, ultraviolet light, or any combination thereof. For example, the illuminators 138 can emit white light, blue light (e.g., indochine blue light), red light, green light, ultraviolet light, near infrared light, infrared light, or any combination thereof. For example, the first, second, third, and forth illuminators 138a, 138b, 138c, and 138d can each emit white light. As another example, the first illuminator 138a can emit white light, the second illuminator 138b can emit blue light (e.g., indochine blue light), the third illuminator 138c can emit red light, and the fourth illuminator 138d can emit green light. The blue light (e.g., indochine blue light), the red light, and/or the green light can be useful, for example, for perfusion visualization.

All methods of optical imaging collect backscattered photons from the mucosa. Conventional endoscopy observes reflected visible light (400-700 nm) from the mucosal surface. However, the light spectrum extends to shorter wavelengths in the ultraviolet (UV) and longer wavelengths in the near-infrared (NIR) that also can be used for endoscopic imaging (FIG. 2). UV and blue light are absorbed by biomolecules to produce fluorescence. The visible band is dominated by hemoglobin absorption and thus has relatively short penetration depths, typically <100 μm, useful for imaging. NIR light is much less sensitive to tissue scattering and hemoglobin absorption20 and thus can usually penetrate <1000 μm through the mucosa. Video endoscopes use charge-coupled device (CCD) detectors that are sensitive to both visible and NIR light. Also, light can undergo elastic or inelastic scattering (FIG. 3), in which the returning photons have the same or longer wavelength as that of the incident, respectively. In addition, ballistic photons return through the tissue without additional scattering events and are useful for deep-tissue imaging, whereas diffuse photons return after several scattering events and are useful for measuring fine morphological structures. In addition to imaging methods, point detection techniques can be used to collect molecular information during endoscopy with optical fiber probes inserted through the instrument channel. These methods have the potential to be extended to imaging. A description of the basic mechanism of how light interacts with tissue and the potential clinical use of each optical biopsy method discussed in this review is provided in Table 2. The figures and tables mentioned in this paragraph can be found in Clinical Gastroenterology and Hepatology, Optical biopsy: A new frontier in endoscopic detection and diagnosis Sep. 1, 2004, Volume 2, Issue 9, Pages744-753 by Thomas D. Wang and Jacques Van Dam, the content of which are herein incorporated by reference in its entirety for all purposes.

The illuminators 138 can be individually or collectively flashed (also referred to as strobed) at different rate. Flashing the illuminators 138 (e.g., LED lights) at particular rates can be useful for stroboscopy, for example, so that the device 100 can be used to look at the movement of body structures, including, for example, vocal chords. One, some, or all of the illuminators 138 can be strobe lights. One, some, or all of the illuminators 138 can have a strobe function and/or a non-strobe function. When the strobe function of the illuminators 138 is activated, the illuminators 138 can emit interrupted light, for example, by flashing. When the non-strobe function of the illuminators 138 is activated, the illuminators 138 can emit uninterrupted light, for example, without flashing.

A strobe light (periodically interrupted light) can be used to visualize a rapidly moving object. If the object(s) (e.g., vocal folds) is/are moving in a relatively periodic (repetitive) fashion, the object(s) will appear to move in slow motion if the frequency of the strobe light is slightly different than the frequency of vibration (e.g., vocal frequency=200 Hz; strobe light frequency=195 Hz). If the frequency of the strobe light is the same as the frequency of vibration of the moving object(s), then the resulting image will appear to be a still image or “freeze frame.” Both of these views are available using the device 100. The device 100 can be used, for example, for the stroboscopic evaluation of the larynx, though the key characteristics of vocal fold vibration (e.g., closure pattern, symmetry of vibration, and mucosal wave—see subsequent section) are all observed and described during a “slow-motion” view (e.g., by flashing strobing or flashing the illuminators 138).

FIG. 55 illustrates that the camera 114 can have a polygonal perimeter. As another example, the camera 114 can have a cylindrical shape as shown in FIGS. 53 and 54.

FIG. 56 illustrates a side view of the deflectable section 180ds in FIG. 46.

The deflectable section 108ds can have 1-20 or more hinges 192, including every 1 hinge increment within this range (e.g., 1 hinge, 2 hinges, 3 hinges, 4 hinges, 10 hinges, 20 hinges). For example, FIG. 56 illustrates that the deflectable section 108ds can have 3 hinges 192, including, for example, a first hinge 192a, a second hinge 192b, a third hinge 192c, or any combination thereof.

The hinges 192 can separate the deflectable section 108ds into 2-20 segments 214, including every 1 segment increment within this range (e.g., 2 segments, 3 segments, 4 segments, 10 segments, 20 segments). For example, FIG. 56 illustrates that the deflectable section 108ds can have four segments 214, including, for example, a first segment 214a, a second segment 214b, a third segment 214c, a fourth segment 214d, or any combination thereof. The first hinge 192a can be between the first and second segments 214a, 214b, the second hinge 192b can be between the second and third segments 214b, 214c, and the third hinge 192c can be between the third and fourth segments 214c, 214d.

The hinges 192 can connect (e.g., fixedly connect or removably connect) adjacent sections 214 to each other. The hinges 192 can be any type of hinge, for example, living hinges, ball and socket hinges, or any combination thereof. The hinges 192 can be, for example, living hinges of the body 106 and/or of the deflectable section 108ds. For example, FIG. 56 illustrates that the hinges 192 can be living hinges of the deflectable section 108ds. As another example, the sections 214 can be separate pieces that can be removably connected to each other with the hinges 192. In such cases, the deflectable section 108ds can be modular, whereby the length of the deflectable section 108ds can be increased and/or decreased by adding and/or removing sections 214 from the deflectable section 108ds, respectively. The segments 214 can be removably connectable to each other, for example, at the hinges 192. As another example, the deflectable section 108ds may not be modular, whereby the sections 214 can be separate pieces that can be fixedly connected to each other via the hinges 192.

The segments 214 can be integrated with each other. For example, FIG. 56 illustrates that adjacent segments 214 can be integrated with each other via the hinges 192. The hinges 192 can be the wall of the body 106 and/or of the deflectable section 108ds. FIG. 56 illustrates, for example, that the hinges 192 can be the wall of the deflectable section 108ds. The hinges 192 can be formed, for example, by the wall of the body and/or the deflectable section 108ds. FIG. 56 illustrates, for example, that the hinges 192 can be formed by the wall of the deflectable section 108ds. As another example, for variations in which the deflectable section 108ds is a distal end of the body 106, the hinges 192 can be the wall of the body 106. In such cases, the hinges 192 can be formed, for example, by the wall of the body 106. The deflectable section 108ds can be one or multiple pieces. FIG. 56 illustrates, for example, that the deflectable section can be a single piece having segments 214 that are integrated with each other, for example, via the hinges 192. As another example, the deflectable section 108ds can be multiple pieces, whereby the segments 214 can be separate pieces that can be connected (e.g., fixedly connected, removably connected) to each other, for example, via the hinges 192.

The proximal most segment 214 (e.g., the first segment 214a) can be attached to or integrated with the body 106. For example, FIG. 56 illustrates that the first segment 214a can be removably attachable to the body 106. As another example, the first segment 214a can be integrated with the body 106, whereby the first segment 214a can be the distal end of the body 106.

Th distal most segment 214 (e.g., the fourth segment 214d) can be attached to or integrated with the camera 114. For example, FIG. 56 illustrates that the fourth segment 214d can be removably attachable to the camera 114. As another example, the fourth segment 214d can be fixedly attached to the camera 114 such that the deflectable section 108ds and the camera 114 can form a subassembly that can be removably attached to or integrated with the body 106. As another example, the camera 114 can be, for example, the distal end of the body 106 with camera components (e.g., the illuminators 138, the lens 139) attached to it.

The segments 214 can be the same size and/or shape as each other, different sizes and/or shapes than each other, or any combination thereof. For example, FIG. 56 illustrates that the proximal most segment 214 (e.g., the first segment 214a), the middle segments 214 (e.g., the second and third segments 214b, 214c), and the distal most segment (e.g., the fourth segment 214d) can have the relative sizes and shapes shown. FIG. 56 illustrates, for example, that the first segment 214a can be larger than the fourth segment 214d, that the first and fourth segments 214a, 214d can be larger than the middle segments (e.g., the second and third segments 214b, 214c), that the middle segments can have the same size and shape as each other (e.g., the second segment 214b can have the same size and/or shape as the third segment 214c), or any combination thereof.

FIG. 56 illustrates that a center longitudinal axis A1 of the body 106 can pass through a center of the hinges 192. FIG. 56 illustrates, for example, that the hinges 192 can be aligned along the center longitudinal axis A1.

The device 100 can have spaces 216 between adjacent sections 214. The spaces 216 can be, for example, channels, grooves, cavities, and/or openings on the surface of the deflectable section 108ds. For example, FIG. 56 illustrates that the spaces 216 can be notches 122 in the surface of the deflectable section 108ds, whereby the notches 122 can have, for example, a wedge shape. The spaces 216 can be gaps between adjacent sections 214 that can give the sections 214 space to deflect when the actuators 112 are tensioned to deflect the deflectable section 108ds, for example, into a curve. The surface of the body 6 and/or the deflectable section 108ds can have undulations on the surface. The undulations can, for example, define the spaces 216.

The deflectable section 108ds can have 1-40 or more spaces 216, including every 1 space increment within this range (e.g., 1 space, 2 spaces, 6 spaces, 10 spaces, 40 spaces). For example, FIG. 56 illustrates that the deflectable section 108ds can have 6 spaces 216, including, for example, a first space 216a, a second space 216b, a third space 216c, a fourth space 216d, a fifth space 216e, a sixth space 216f, or any combination thereof.

The hinges 192 can be adjacent the spaces 216. A hinge 192 can be, for example, between spaces 216. For example, FIG. 56 illustrates that the first hinge 192a can be between the first and fourth spaces 216a, 216d, that the second hinge 192b can be between the second and fifth segments 216b, 216e, that the third hinge 192c can be between the third and sixth segments 216c, 216f, or any combination thereof.

A space 216 can be on one or multiple sides of a hinge 192. For example, FIG. 56 illustrates that the spaces 216 can be on two sides of the hinges 192. For example, FIG. 56 illustrates that the first, second, and third spaces 216a, 216b, 216c can be on a first side of the first, second, and third hinges 192a, 192b, 192c, respectively. As another example, FIG. 56 illustrates that the fourth, fifth, and sixth spaces 216d, 216e, 216f can be on a second side of the first, second, and third hinges 192a, 192b, 192c, respectively. FIG. 56 illustrates, for example, that the first side of the hinges 192 can be opposite the second side of the hinges 192. For example, FIG. 56 illustrates that the first, second, and third spaces 216a, 216b, 216c can be on a first side of the center longitudinal axis A1, and that the fourth, fifth, and sixth spaces 216d, 216e, 216f can be on a second side of the center longitudinal axis A1, whereby the first side of the center longitudinal axis A1 can be opposite the second side of the center longitudinal axis A1. For example, FIG. 56 illustrates that the first space 216a can be opposite the fourth space 216d, that that the second space 216b can be opposite the fifth space 216e, and that the third space 216c can be opposite the sixth space 216f.

A space 216 can be on one or multiple sides of the deflectable section 108ds. For example, FIG. 56 illustrates that the spaces 216 can be on two sides of the deflectable section 108ds. For example, FIG. 56 illustrates that the first, second, and third spaces 216a, 216b, 216c can be on a first side (e.g., a first lateral side) of the deflectable section 108ds. As another example, FIG. 56 illustrates that the fourth, fifth, and sixth spaces 216d, 216e, 216f can be on a second side (e.g., a second lateral side) of the deflectable section 108ds. FIG. 56 illustrates, for example, that the first side of the deflectable section 108ds can be opposite the second side of the deflectable section 108ds. For example, FIG. 56 illustrates that the first, second, and third spaces 216a, 216b, 216c can be on a first side (e.g., a first lateral side) of the center longitudinal axis A1, and that the fourth, fifth, and sixth spaces 216d, 216e, 216f can be on a second side (e.g., a second lateral side) of the center longitudinal axis A1, whereby the first side of the center longitudinal axis A1 can be opposite the second side of the center longitudinal axis A1. FIG. 56 illustrates that the first space 216a can be opposite the fourth space 216d, that that the second space 216b can be opposite the fifth space 216e, and that the third space 216c can be opposite the sixth space 216f.

The spaces 216 can have any size and/or shape. For example, FIG. 56 illustrates that the spaces 216 can have the relative sizes and shapes shown. FIG. 56 illustrates, for example, that the spaces 216 can increase in length radially outward from the center longitudinal axis A1 such that the spaces 216 can be shorter closer to the center longitudinal axis A1 and longer farther from the center longitudinal axis A1. For example, FIG. 56 illustrates that the spaces 216 can have a first length a first distance (e.g., a first radius) from the center longitudinal axis A1, and can have a second length a second distance (e.g., a second radius) from the center longitudinal axis A1, whereby the second distance can be greater than the first distance. FIG. 56 illustrates, for example, that the spaces 216 can have a wedge shape.

The deflectable section 108ds can have surfaces 218. The surfaces 218 can define the spaces 216. For example, the surfaces 218 can define the notches 122. The deflectable section 108ds can have 2-80 or more surfaces 218, including every 1 surface increment within this range (e.g., 2 surfaces, 10 surfaces, 12 surfaces, 20 surfaces, 80 surfaces). For example, FIG. 56 illustrates that the deflectable section 108ds can have 12 surfaces 218, including, for example, a first surface 218a, a second surface 218b, a third surface 218c, a fourth surface 218d, a fifth surface 218e, a sixth surface 218f, a seventh surface 218g, an eighth surface 218h, a ninth surface 218i, a tenth surface 218j, an eleventh surface 218k, a twelfth surface 218l, or any combination thereof.

When the deflectable section 108ds is in a straight configuration (e.g., a non-deflected configuration), adjacent surfaces 218 can face each other. For example, FIG. 56 illustrates that when the deflectable section 108ds is in a straight configuration (e.g., a non-deflected configuration), the first and second surfaces 218a, 218b can face each other, the third and fourth surfaces 218c, 218d can face each other, the fifth and sixth surfaces 218e, 218f can face each other, the seventh and eighth surfaces 218g, 218h can face each other, the ninth and tenth surfaces 218i, 218j can face each other, the eleventh and twelfth surfaces 218k, 218l can face each other, or any combination thereof.

FIG. 56 illustrates that the hinges 192 can be at the base of the spaces 216. The deflectable section 108ds can flex about the hinges 192, for example, as the control 126 is rotated (e.g., in the first direction 188a or in the second direction 188b). The hinges 192 and the spaces 216 can allow the deflectable tip 108ds to flex when the actuators 112 are tensioned (e.g., pulled in direction 116b). The hinges 192 and the spaces 216 can thereby assist in articulation of the deflectable section 108ds.

The segments 214 can pivot about the hinges 192, for example, when the actuators 112 are tensioned (e.g., pulled in direction 116b). The segments 214 can rotate about the hinges 192, for example, when the actuators 112 are tensioned (e.g., pulled in direction 116b). For example, when the first actuator 112a is tensioned (e.g., is pulled in direction 116b), the deflectable section 108ds can move in a first direction 120a to form various arcs, and when the second actuator 112b is tensioned (e.g., is pulled in direction 116b), the deflectable section 108ds can move in a second direction 120b to form various arcs. As another example, when a first end of an actuator 112 is tensioned (e.g., is pulled in direction 116b), the deflectable section 108ds can move in a first direction 120a to form various arcs, and when a second end of the actuator 112 is tensioned (e.g., is pulled in direction 116b), the deflectable section 108ds can move in a second direction 120b to form various arcs.

The spaces 216 can be openable and closeable. When a space 216 opens, a dimension (e.g., length) of the space 216 can increase, and when a space 216 closes, the dimension (e.g., length) of the space 216 can decrease. For example, when the deflectable section 108ds is articulated in the first direction 120a, the spaces 216 on the first side of the center longitudinal axis A1 (e.g., the first space 216a, the second space 216b, and/or the third space 216c) can partially close or fully close, and the spaces 216 on the second side of the center longitudinal axis A1 (e.g., the fourth space 216d, the fifth space 216e, and/or the sixth space 2160 can partially open or fully open. As another example, when the deflectable section 108ds is articulated in the second direction 120b, the spaces 216 on the second side of the center longitudinal axis A1 (e.g., the fourth space 216d, the fifth space 216e, and/or the sixth space 2160 can partially close or fully close, and the spaces 216 on the first side of the center longitudinal axis A1 (e.g., the first space 216a, the second space 216b, and/or the third space 216c) can partially open or fully open.

The spaces 216 can open and close, for example, by the sections 214 moving (e.g., rotating) toward each other and away from each other as the deflectable section 108ds is articulated. The spaces 216 can open and close, for example, by adjacent surfaces 218 moving (e.g., rotating) toward each other and away from each other as the deflectable section 108ds is articulated.

For example, when the deflectable section 108ds is articulated in the first direction 120a, the adjacent surfaces 218 on the first side of the center longitudinal axis A1 (e.g., the first, second, third, fourth, fifth, and sixth surfaces 218a, 218b, 218c, 218d, 218e, 2180 can move toward each other, and the adjacent surfaces 218 on the second side of the center longitudinal axis A1 (e.g., the seventh, eighth, ninth, tenth, eleventh, and twelfth surfaces 218g, 218h, 218i, 218j, 218k, 218l) can move away each other. For example, FIG. 56 illustrates that when the deflectable section 108ds is articulated in the first direction 120a, the second surface 218b can move toward the first surface 218a, the fourth surface 218d can move toward the third surface 218c, the sixth surface 218f can move toward the fifth surface 218e, the eighth surface 218h can move away from the seventh surface 218g, the tenth surface 218j can move away from the ninth surface 218i, and/or the twelfth surface 218l can move away from the eleventh surface 218k, or any combination thereof.

As another example, when the deflectable section 108ds is articulated in the second direction 120b, the adjacent surfaces 218 on the second side of the center longitudinal axis A1 can move toward each other, and the adjacent surfaces 218 on the first side of the center longitudinal axis A1 can move away each other. For example, FIG. 56 illustrates that when the deflectable section 108ds is articulated in the second direction 120b, the second surface 218b can move away from the first surface 218a, the fourth surface 218d can move away from the third surface 218c, the sixth surface 218f can move away from the fifth surface 218e, the eighth surface 218h can move toward the seventh surface 218g, the tenth surface 218j can move toward the ninth surface 218i, and/or the twelfth surface 218l can move toward the eleventh surface 218k, or any combination thereof.

When a space 216 becomes fully closed, adjacent surfaces 218 can contact each other. When a space 216 becomes fully closed, adjacent surfaces 218 can be parallel to each other.

When the deflectable section 108ds is in a straight configuration (e.g., a non-deflected configuration), an angle 220 can be between adjacent surfaces 218. When the deflectable section 108ds is in a straight configuration (e.g., a non-deflected configuration), the angle 220 between adjacent surfaces 218 can be, for example, 10 degrees to 120 degrees, or more narrowly, 10 degrees to 90 degrees, or more narrowly still, 30 degrees to 90 degrees, including every 1 degree increment within these ranges (e.g., 10 degrees, 30 degrees, 45 degrees, 90 degrees, 120 degrees). For example, FIG. 56 illustrates that when the deflectable section 108ds is in a straight configuration (e.g., a non-deflected configuration), the angle 220 between adjacent surfaces 218 can be 45 degrees.

The angles 220 can include a first angle 220a between the first surface 218a and the second surface 218b, a second angle 220b between the third surface 218c and the fourth surface 218d, a third angle 220c between the fifth surface 218e and the sixth surface 218f, a fourth angle 220d between the seventh surface 218g and the eighth surface 218, a fifth angle 220e between the ninth surface 218i and the tenth surface 218j, a sixth angle 220f between the eleventh surface 218k and the twelfth surface 218l, or any combination thereof. The angles 220 between each pair of adjacent surfaces 218 can be the same as or different from each other. For example, FIG. 56 illustrates that the angles 220 can be the same such that the first angle 220a, the second angle 220b, the third angle 220c, the fourth angle 220d, the fifth angle 220e, and the sixth angle 220f can each be the same, for example, 45 degrees.

The angle 220 can increase when the spaces 216 open (e.g., partially open or fully open), for example, when adjacent surfaces 218 move away from each other. The angle 220 can increase for example, by 1 degree to 45 degrees, including every 1 degree increment within this range (e.g., 1 degree, 15 degrees, 30 degrees, 45 degrees).

The angle can decrease when the spaces 216 close (e.g., partially close or fully close), for example, when adjacent surfaces 218 move toward each other. The angle 220 can decrease for example, by 1 degree to 45 degrees, including every 1 degree increment within this range (e.g., 1 degree, 15 degrees, 30 degrees, 45 degrees).

FIG. 56 illustrates that when the deflectable section 108ds is in a straight configuration (e.g., a non-deflected configuration), the spaces 216 can be partially open, whereby the angle 220 can be 45 degrees.

When the deflectable section 108ds is fully articulated in the first direction 120a (e.g., when the first actuator 112a or the first end of an actuator 112 is fully tensioned in direction 116b), one, some, or all of the spaces 216 on the first side of the center longitudinal axis A1 can be fully closed, and one some, or all of the spaces 216 on the second side of the center longitudinal axis A1 can be fully open.

When the deflectable section 108ds is fully articulated in the second direction 120b (e.g., when the second actuator 112b or the second end of the actuator 112 is fully tensioned in direction 116b), one, some, or all of the spaces 216 on the second side of the center longitudinal axis A1 can be fully closed, and one some, or all of the spaces 216 on the first side of the center longitudinal axis A1 can be fully open.

When a space 216 is fully open, the opposite space 216 can be fully closed and vice versa. For example, when the first space 216a is fully closed (e.g., when the first surface 218a is in contact with the second surface 218b), the fourth space 216d can be fully open. As another example, when the first space 216a is fully open (e.g., when the seventh surface 218g is in contact with the eight surface 218h), the first space 216a can be fully open.

The maximum number of degrees by which the angle 220 can increase or decrease can be, for example, the number of degrees of the angle 220 when the deflectable section 108ds is in a straight configuration (e.g., a non-deflected configuration). For example, FIG. 56 illustrates that the angle 220 can be 45 degrees such that the maximum by which the angle can increase can be 45 degrees and such that the maximum by which the angle can decrease can be 45 degrees. FIG. 56 illustrates that when the deflectable section 108ds is in a straight configuration (e.g., a non-deflected configuration), the spaces 216 can be partially open and the angle 220 can be 45 degrees. In such a case, when the deflectable section 108ds is fully articulated in the first direction 120a (e.g., when the first actuator 112a or the first end of an actuator 112 is fully tensioned), the spaces 216 on the first side of the center longitudinal axis A1 (e.g., the first space 216, the second space 216b, and the third space 216c) can be fully closed such that the angles 220 on the first side of the center longitudinal axis A1 (e.g., the first angle 220a, the second angle 220b, and the third angle 220c) can be 0 degrees, and the spaces 216 on the second side of the center longitudinal axis A1 (e.g., the fourth space 216d, the fifth space 216e, and the sixth space 2160 can be fully open such that the angles 220 on the second side of the center longitudinal axis A1 (e.g., the fourth angle 220d, the fifth angle 220e, and the sixth angle 2200 can be 90 degrees.

As another example, the deflectable section 180ds may not have any hinges 192 (e.g., see FIGS. 11-14).

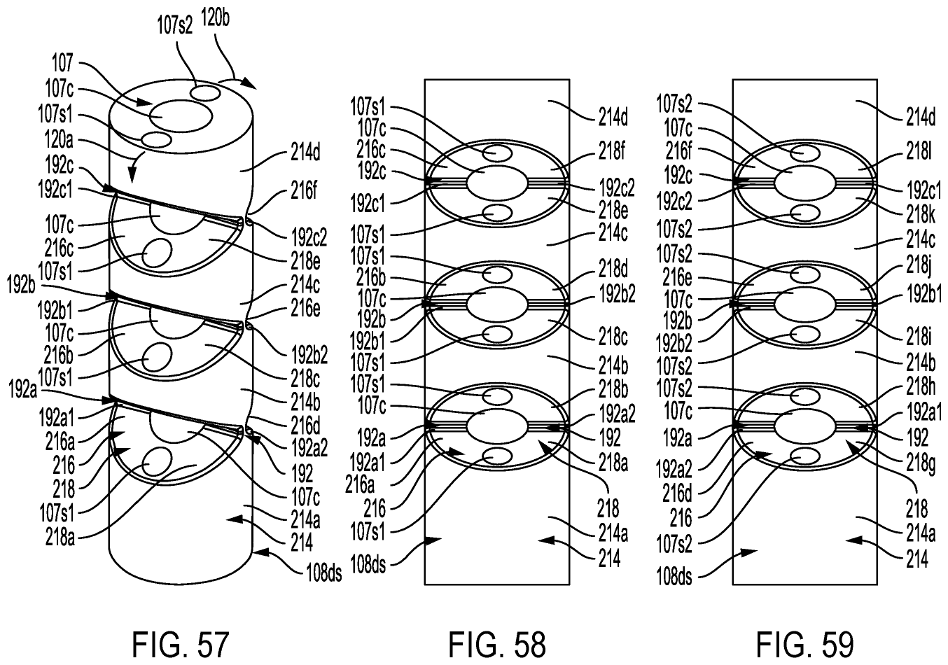

The device 100 can have, for example, 1-10 lumens, including every 1 lumen increment within this range (e.g., 1 lumen, 2 lumens, 3 lumens, 10 lumens). The lumens 107 can be in the body 106, the deflectable section 108ds, and/or the camera 114. The lumens 107 can extend through the body 106, through the deflectable section 108ds, and/or through the camera 114. For example, FIG. 57 illustrates that the deflectable section 108ds can have three lumens 107, including, for example, a central lumen 107c and two side lumens 107s. The side lumens 107s can include, for example, a first side lumen 107s1 and a second side lumen 107s2. The first and second side lumens 107s1, 107s2 can be on opposite sides of the central lumen 107c. The actuators 112 and/or the connector 210 can extend through the lumens 107. For example, the first actuator 112a can extend through the first side lumen 107s1, the second actuator 112b can extend through the second side lumen 107s2, and the connector 200 can extend through the central lumen 107c. As another example, a first end of an actuator 112 can extend through the first side lumen 107s1, a second end of the actuator 112 can extend through the second side lumen 107s2, and the connector 200 can extend through the central lumen 107c. The lumens 107 can have the same size or different sizes. For example, FIG. 57 illustrates that the central lumen 107c can be larger than the side lumens 107s and that the first and second side lumens 107s1, 107s2 can have the same size. The central lumen 107c can pass through a center of the body 106, deflectable section 108ds, and/or the camera 114. As another example, the central lumen 107c may not be a central lumen such that the central lumen 107c can be positioned anywhere in the body 106, the deflectable section 108ds, and/or the camera 114.

FIG. 57 illustrates that the lumens 107 can extend through the segments 214, for example, through the first segment 214a, through the second segment 214b, through the third segment 214c, through the fourth segment 214d, or through any combination thereof. FIG. 57 illustrates that the lumens 107 can extend through the surfaces 218. For example, FIG. 57 illustrates that the first side lumen 107s1 can extend through the first through sixth surfaces 218a-218f, that the second side lumen 107s2 can extend through the seventh through twelfth surfaces 218g-218l, and that the central lumen 107c can extend through the first through twelfth surfaces 218a-218l.

FIG. 57 illustrates that a lumen 107 (e.g., the central lumen 107c) can extend through the hinges 192, for example, through the first hinge 192a, through the second hinge 192b, through the third hinge 192c, or through any combination thereof. The hinges 192 can have, for example, a hinge first side and a hinge second side. The hinge first side can be on a first side (e.g., a first lateral side) of the deflectable section 108ds and the hinge second side can be on a second side (e.g., a second lateral side) of the deflectable section 108ds. The hinge first side can be on a first side of the lumen 107 (e.g., the central lumen 107c) and the hinge second side can be on a second side of the lumen 107 (e.g., the central lumen 107c). FIG. 57 illustrates, for example, that the first hinge 192a can have a first hinge first side 192a1 and a first hinge second side 192a2, that the second hinge 192b can have a second hinge first side 192b1 and a second hinge second side 192b2, and that the third hinge 192c can have a third hinge first side 192c1 and a third hinge second side 192c2. As another example, FIG. 57 illustrates that the central lumen 107c can separate or split each of the hinges 192 into two hinges such that there can be two first hinges 192a, two second hinges 192b, and two third hinges 192c.

FIG. 58 illustrates that the first side lumen 107s1 can extend through the first through sixth surfaces 218a-218f, and that the central lumen 107c can extend through the first through twelfth surfaces 218a-218l.

FIG. 59 illustrates that the second side lumen 107s2 can extend through the seventh through twelfth surfaces 218g-218l, and that the central lumen 107c can extend through the first through twelfth surfaces 218a-218l.

FIGS. 56-59 illustrate, for example, that the first through sixth surfaces 218a-218f can each have two openings (e.g., a first opening which can be an opening of the first side lumen 107s1 and a second opening which can be an opening of the central lumen 107c), and that the seventh through twelfth surfaces 218g-218l can each have two openings (e.g., a first opening which can be an opening of the second side lumen 107s2 and a second opening which can be an opening of the central lumen 107c).

FIGS. 56-59 illustrate that the surfaces 218 can be flat surfaces. As another example, the surfaces 218 can be curved surfaces.

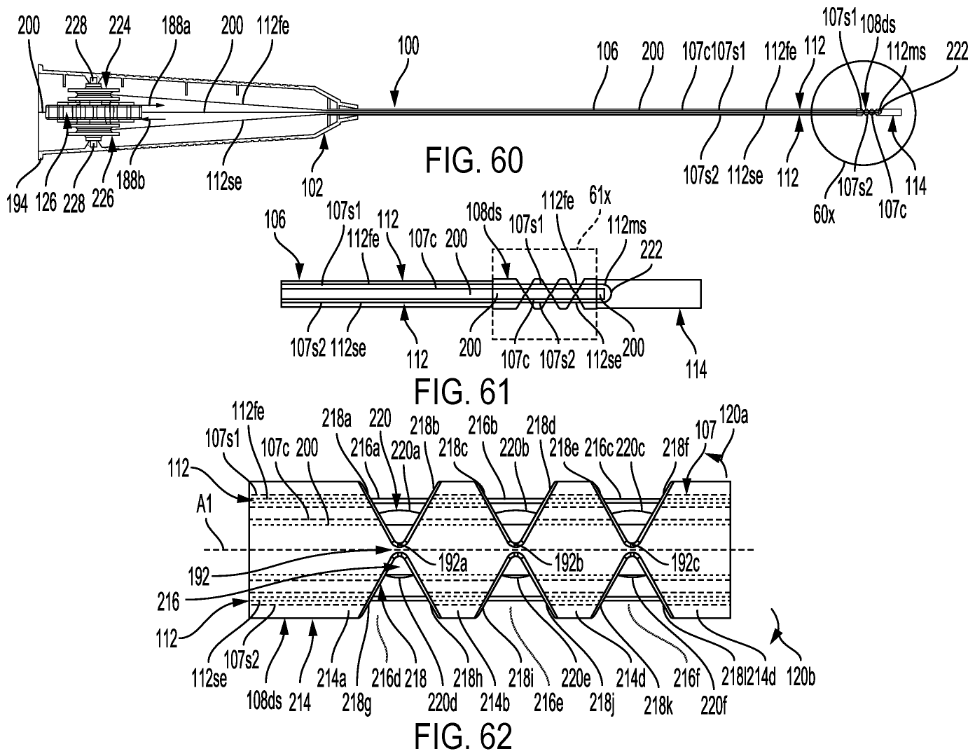

FIG. 60 illustrates the device 100, for example, of FIG. 44 with the handle 102 distal the ridge 194, the body 106, the deflectable section 108ds, and the camera 114 shown transparent so that the actuator 112 and the connector 200 can be seen.

FIG. 60 illustrates that the connector 200 can extend from the camera 114, through the deflectable section 108ds, through the body 106, and through the handle 102 to the connector 202. For example, FIG. 60 illustrates that the connector 200 can extend from the camera 114 through the central lumen 107c in the deflectable section 108ds and through the central lumen 107c in the body 106.

FIG. 60 illustrates that the device 100 can have an actuator 112, for example, one actuator 112 having an actuator first end 112fe (also referred to as a first end of the actuator) and an actuator second end 112se (also referred to as a second end of the actuator). FIG. 60 illustrates that the actuator 112 can extend through the first side lumen 107s1 in the body 106 and in the deflectable section 108ds, through a lumen 222 in the camera 114, and through the second side lumen 107s2 in the body 106 and in the deflectable section 108ds. For example, FIG. 60 illustrates that the actuator first end 112fe can extend through the first side lumen 107s1 in the body 106 and in the deflectable section 108ds, that an actuator middle section 112ms (also referred to as a middle of the actuator) can extend through lumen 222 in the camera 114, and that the actuator second end 112se can extend through the second side lumen 107s2 in the body 106 and in the deflectable section 108ds.

FIG. 60 illustrates that the control 126 can be attached to a first pulley 224 and that the control 126 can be attached to a second pulley 226. The first and second pulleys 224, 226 can be attached to the handle 102, for example, via a pin 228. FIG. 60 illustrates that when the control 126 (e.g., wheel) is rotated in the first direction 188a, the first and second pulleys 224, 226 can rotate in the first direction 188a with the pin 228 or about the pin 228. FIG. 60 illustrates that when the control 126 (e.g., wheel) is rotated in the second direction 188b, the first and second pulleys 224, 226 can rotate in the second direction 188b with the pin 228 or about the pin 228. The pin 228 can pass through the control 226 such that the control 126 can rotate with the pin 228 or about the pin 228 when the control 126 is rotated in the first and second directions 188a, 188b.

FIG. 60 illustrates, for example, that the actuator first end 112fe can be windable and unwindable on the first pulley 224. The actuator first end 112fe can be attached to the first pulley 224. For example, FIG. 60 illustrates that a proximal end of the actuator first end 112fe can be attached to the first pulley 224. The actuator first end 112fe can be attached to the control 126, for example, via the first pulley 224.

FIG. 60 illustrates, for example, that the actuator second end 112se can be windable and unwindable on the second pulley 226. The actuator second end 112se can be attached to the second pulley 226. For example, FIG. 60 illustrates that a proximal end of the actuator second end 112se can be attached to the second pulley 224. The actuator second end 112se can be attached to the control 126, for example, via the second pulley 224.

FIG. 60 illustrates that the actuator first and second ends 112fe, 112se can diverge from each other inside the handle 102, for example, as the actuator first and second ends 112fe, 112se extend toward the first and second pulleys 224, 226, respectively.

FIG. 60 illustrates that the actuator 112 can loop around the distal end of the device 100. For example, FIG. 60 illustrates that the lumen 222 can connect the first side lumen 107s1 to the second side lumen 107s2 such that the actuator 112 (e.g., via the actuator middle section 112ms) can loop around the distal end of the device 100 by extending through the lumen 222.

The actuator middle section 112ms can be attached to the deflectable section 108ds and/or to the camera 114. For example, FIG. 60 illustrates that the actuator middle section 112ms can be attached to the camera 114. Half of the actuator middle section 112ms can be a distal portion of the actuator first end 112fe, and half of the actuator middle section 112ms can be a distal portion of the actuator second end 112se. By attaching the actuator middle section 112ms (e.g., the distal ends of the actuator first and second ends 112fe, 112se) to the camera 114, the actuator first and second ends 112fe, 112se can be tensioned by actuating (e.g., rotating) the control 126. The actuator middle section 112ms can be attached to the camera 114, for example, inside the lumen 222.

FIG. 60 illustrates, for example, that the actuator first end 112fe can be wrapped around the first pulley 224 in a first direction, and that the actuator second end 112se can be wrapped around the second pulley 226 in a second direction, where the first direction can be opposite the second direction. Having the first and second pulleys 224, 226 wrapped in opposite directions can allow the first pulley 224 to wrap (e.g., tension) the actuator first end 112fe as the second pulley 226 unwraps (e.g., detensions) the actuator second end 112se. Having the first and second pulleys 224, 226 wrapped in opposite directions can allow the second pulley 226 to wrap (e.g., tension) the actuator second end 112se as the first pulley 224 unwraps (e.g., detensions) the actuator first end 112fe. For example, FIG. 60 illustrates that when the control 126 (e.g., wheel) is rotated in the first direction 188a, the first and second pulleys 224, 226 can rotate in the first direction 188a such that the first pulley 224 wraps (e.g., tensions) the actuator first end 112fe and the second pulley 226 unwraps (e.g., detensions) the actuator second end 112se. This can deflect the tip 108 (e.g., the deflectable section 108ds and/or the camera 114) in the first direction 120a, for example, as shown in FIG. 67a. As another example, FIG. 60 illustrates that when the control 126 (e.g., wheel) is rotated in the second direction 188b, the first and second pulleys 224, 226 can rotate in the second direction 188b such that the first pulley 224 unwraps (e.g., detensions) the actuator first end 112fe and the second pulley 226 wraps (e.g., tensions) the actuator second end 112se. This can deflect the tip 108 (e.g., the deflectable section 108ds and/or the camera 114) in the second direction 120b, for example, as shown in FIG. 67b.

FIG. 61 illustrates a closeup of section 60x in FIG. 60. FIG. 61 illustrates that the connector 200 can have a larger width (e.g., diameter) than the actuator 112. FIG. 61 illustrates that the actuator middle section 112ms can be the portion of the actuator 112 that is in the camera 114. FIG. 61 illustrates that the actuator middle section 112ms can be the portion of the actuator 112 that is attached to the camera 114. As another example, the actuator middle section 112ms can be the portion of the actuator 112 that is attached to the deflectable section 108ds (e.g., for variations in which the actuator 112 does not extend into the camera 114). FIG. 61 illustrates that at the deflectable section is deflected in the first direction 120a, the segments 214 of the deflectable section 108ds can translate (e.g., slide) over the actuator first end 112fe and can translate (e.g., slide) over the actuator second end 112se. FIG. 61 illustrates that at the deflectable section is deflected in the second direction 120b, the segments 214 of the deflectable section 108ds can translate (e.g., slide) over the actuator first end 112fe and can translate (e.g., slide) over the actuator second end 112se. FIG. 61 illustrates that at the deflectable section is deflected in the first direction 120a or the second direction 120b, the actuator middle section 112ms can remain fixed to the camera 114 such that the camera 114 may not translate (e.g., slide) over the actuator middle section 112ms. FIG. 61 illustrates that the connector 200 can extend into the camera 114. FIG. 61 illustrates that the distal terminal end of the connector 200 can be in the camera 114.

FIG. 62 illustrates a closeup of section 61x in FIG. 61. For example, FIG. 62 illustrates the deflectable section 108ds of FIG. 56 with the actuator first end 112fe in the first side lumen 107s1, the actuator second end 112se in the second side lumen 107s2, and the connector 200 in the central lumen 107c. FIG. 62 illustrates, for example, a closeup of the deflectable section 108ds of FIG. 60 with the actuator 112 and the connector 200 extending through the deflectable section 108ds.

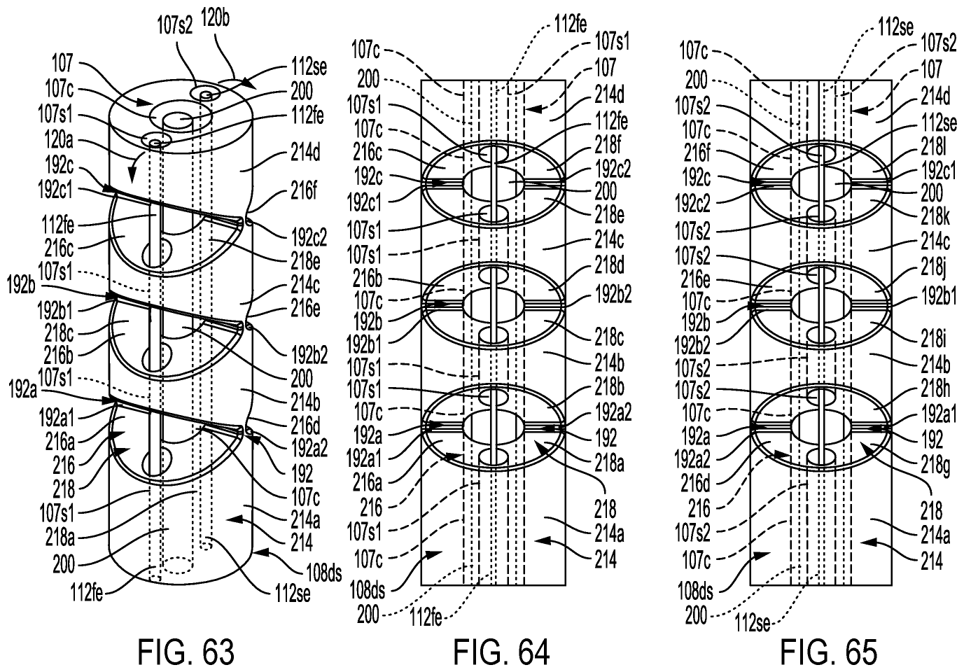

FIG. 63 illustrates the deflectable section 108ds of FIG. 57 with the actuator first end 112fe in the first side lumen 107s1, the actuator second end 112se in the second side lumen 107s2, and the connector 200 in the central lumen 107c. FIG. 63 illustrates, for example, a front perspective view of the deflectable section 108ds of FIG. 60 with the actuator 112 and the connector 200 extending through the deflectable section 108ds.

FIG. 64 illustrates the deflectable section 108ds of FIG. 58 with the actuator first end 112fe in the first side lumen 107s1 and the connector 200 in the central lumen 107c. FIG. 64 illustrates, for example, a top view of the deflectable section 108ds of FIG. 60 with the actuator 112 and the connector 200 extending through the deflectable section 108ds.

FIG. 65 illustrates the deflectable section 108ds of FIG. 59 with the actuator second end 112se in the second side lumen 107s2 and the connector 200 in the central lumen 107c. FIG. 65 illustrates, for example, a bottom view of the deflectable section 108ds of FIG. 60 with the actuator 112 and the connector 200 extending through the deflectable section 108ds.

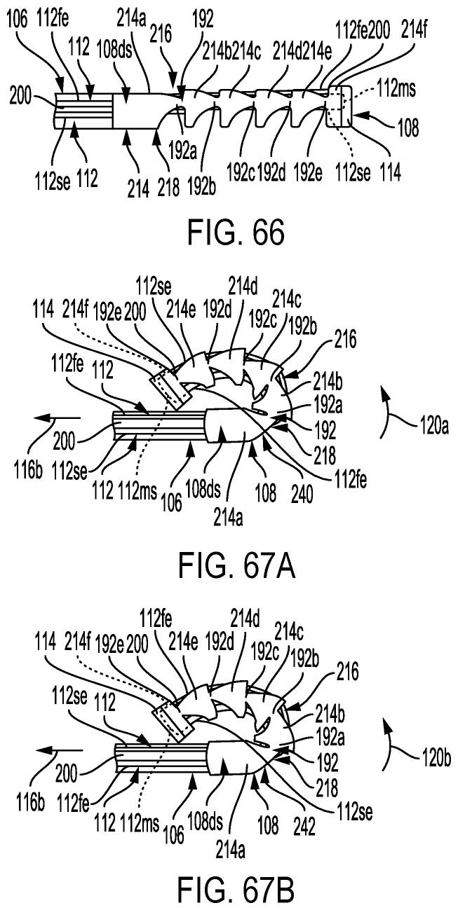

FIG. 66 illustrates that the deflectable section 108ds can have six segments 214, including, for example, the first segment 214a, the second segment 214b, the third segment 214c, the fourth segment 214d, a fifth segment 214e, a sixth segment 214f, or any combination thereof. For example, Figure illustrates that the deflectable section 108ds of FIGS. 56-65 can have a fifth segment 214e and a sixth segment 214f. FIG. 66 illustrates that the camera 114 can be attached to the distal end of the sixth segment 214f. As another example, the deflectable section may only have the first, second, third, and fourth segments 214a, 214b, 214c, and 214d as shown, for example, in FIGS. 56-65, in which case the camera 114 can be attached to the distal end of the fourth segment 214d as shown, for example, in FIGS. 60 and 61. FIGS. 65-66 illustrate that when the deflectable section 108ds is in a straight configuration (e.g., a non-deflected configuration), the actuator first and second ends 112fe, 112se can be in a neutral configuration.

FIG. 66 illustrates that when the deflectable section 108ds is in a straight configuration (e.g., a non-deflected configuration), the spaces 216 can have a neutral configuration. The neutral configuration can be, for example, a partially open configuration. The neutral configuration for each space 216 is also referred to as the neutral open configuration and the first open configuration.

FIG. 67a illustrates that when the actuator first end 112fe is tensioned in direction 116b, for example, by rotating the control 126 (e.g., wheel) in the first direction 188a, the deflectable section 108ds can deflect into a curve 240 by deflecting (e.g., rotating) in the first direction 120a, whereby the spaces 216 on the same side of the deflectable section 108ds as the actuator first end 112fe can be smaller than when the deflectable section 108ds is in the straight configuration (e.g., see FIG. 66), and whereby the spaces 216 on the same side of the deflectable section 108ds as the actuator second end 112se can be larger than when the deflectable section 108ds is in the straight configuration (e.g., see FIG. 66). In other words, FIG. 67a illustrates that the spaces 216 on the radial outside of the curve 240 can be more open than the spaces 216 on the radial inside of the curve 240. FIG. 67a illustrates, for example, that the surfaces 218 on the same side of the deflectable section 108ds as the actuator first end 112fe can be closer to each other when the deflectable section 108ds is in a curved configuration (e.g., articulated in the first direction 120a to have the curve 240) than when the deflectable section 108ds is in the straight configuration (e.g., see FIG. 66). FIG. 67a illustrates, for example, that the surfaces 218 on the same side of the deflectable section 108ds as the actuator second end 112se can be farther from each other when the deflectable section 108ds is in a curved configuration (e.g., articulated in the first direction 120a to have the curve 240) than when the deflectable section 108ds is in the straight configuration (e.g., see FIG. 66).

FIG. 67a illustrates that the actuator first end 112fe can be pulled in direction 116b to cause the distal end of the deflectable section 108ds and the camera 114 to rotate through an angle of 1 degree to 270 degrees, or more narrowly, of 1 degree to 235 degrees, including every 1 degree increment within these ranges (e.g., 1 degree, 180 degrees, 235 degrees, 270 degrees), where a 180 degree rotation can be considered a full retroflexion such that the camera 114 is pointed in an opposite direction than to the direction of the camera 114 when the deflectable section 108ds is in the straight configuration (e.g., see FIG. 66). FIG. 67a illustrates, for example, that the angle can 210 degrees. FIG. 67a illustrates that when the tension on the actuator first end 112de is released, for example, by rotating the control 126 in the second direction 188b, the deflectable section 108ds can become less curved or can return to the non-actuated configuration in FIGS. 65-66.

FIG. 67b illustrates that when the actuator second end 112se is tensioned in direction 116b, for example, by rotating the control 126 (e.g., wheel) in the second direction 188b, the deflectable section 108ds can deflect into a curve 242 by deflecting (e.g., rotating) in the second direction 120b, whereby the spaces 216 on the same side of the deflectable section 108ds as the actuator second end 112se can be smaller than when the deflectable section 108ds is in the straight configuration (e.g., see FIG. 66), and whereby the spaces 216 on the same side of the deflectable section 108ds as the actuator first end 112fe can be larger than when the deflectable section 108ds is in the straight configuration (e.g., see FIG. 66). In other words, FIG. 67b illustrates that the spaces 216 on the radial outside of the curve 242 can be more open than the spaces 216 on the radial inside of the curve 242. FIG. 67b illustrates, for example, that the surfaces 218 on the same side of the deflectable section 108ds as the actuator second end 112se can be closer to each other when the deflectable section 108ds is in a curved configuration (e.g., articulated in the second direction 120b to have the curve 242) than when the deflectable section 108ds is in the straight configuration (e.g., see FIG. 66). FIG. 67b illustrates, for example, that the surfaces 218 on the same side of the deflectable section 108ds as the actuator first end 112fe can be farther from each other when the deflectable section 108ds is in a curved configuration (e.g., articulated in the second direction 120b to have the curve 242) than when the deflectable section 108ds is in the straight configuration (e.g., see FIG. 66).

FIG. 67b illustrates that the actuator second end 112se can be pulled in direction 116b to cause the distal end of the deflectable section 108ds and the camera 114 to rotate through an angle of 1 degree to 270 degrees, or more narrowly, of 1 degree to 235 degrees, including every 1 degree increment within these ranges (e.g., 1 degree, 180 degrees, 235 degrees, 270 degrees), where a 180 degree rotation can be considered a full retroflexion such that the camera 114 is pointed in an opposite direction than to the direction of the camera 114 when the deflectable section 108ds is in the straight configuration (e.g., see FIG. 66). FIG. 67b illustrates, for example, that the angle can 210 degrees. FIG. 67b illustrates that when the tension on the actuator second end 112se is released, for example, by rotating the control 126 in the first direction 188a, the deflectable section 108ds can become less curved or can return to the non-actuated configuration in FIGS. 65-66.

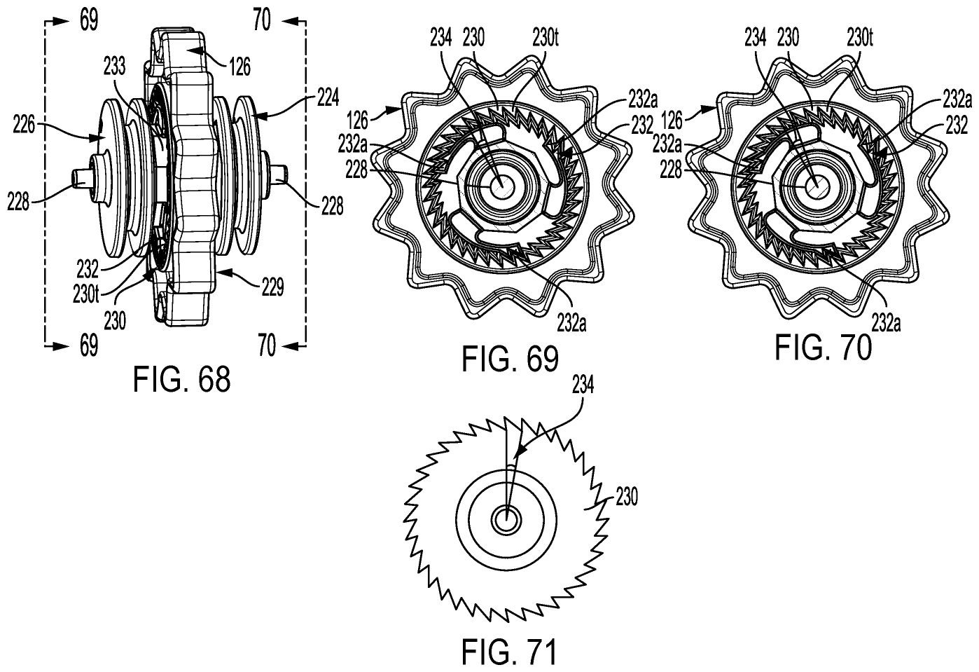

FIG. 68 illustrates that the device 100 can have a ratchet system 229, for example, so that the deflectable section 108ds can be controllably articulated with the control 126. The ratchet system 229 can be inside the handle 102. FIG. 68 illustrates that the ratchet system can include, for example, the control 126, the first pulley 224, the second pulley 226, the pin 228, a gear 230 with teeth 230t, and a catch 232 (also referred to as a pawl 232) that can engage with the teeth 230t. FIG. 68 illustrates that the gear can be a ring gear such that the teeth 230t extend radially inward, for example, toward the longitudinal axis of the pin 228. FIG. 68 illustrates that the device 100 can have a connector 233.

FIG. 68 illustrates that the ratchet system 229 can be a two way ratchet, whereby when the control 126 is rotated in the first direction 188a, the ratchet system 229 can prevent rotation of the control 126 in the second direction 188b, and whereby when the control 126 is rotated in the second direction 188b, the ratchet system 229 can prevent rotation of the control 126 in the first direction 188a. The ratchet system 229 can have, for example, two gears 230 and two catches 232, including a first gear and a second gear. A first catch can engage the first gear. A second catch can engage the second gear. When the control 126 is rotated in the first direction 188a, the first gear and the first catch can be engaged such that the control 126 is prevented from rotating in the second direction 188b. When the control 126 is rotated in the second direction 188b, the second gear and the second catch can be engaged such that the control 126 is prevented from rotating in the first direction 188a.

FIG. 69 illustrates, for example, a side view of the ratchet system 229 without the first pulley 224 viewed from line 69-69 in FIG. 68. FIG. 69 illustrates that the gear 230 (e.g., ring gear) can have 36 teeth, for example, with an angle 234 between each tooth. The angle 234 can be, for example, 360 degrees divided by the number of teeth 230t. FIG. 69 illustrates, for example, that the gear 230 can have 36 teeth 230t such that the angle can be 10 degrees. The gear 230 and the catch 232 shown in FIG. 69 can be the first gear and the first catch. FIG. 69 illustrates that the catch 232a can have one, two, or three arms 232a with teeth (e.g., one or two teeth) that can releasably engage with the teeth 230t.

FIG. 70 illustrates, for example, a side view of the ratchet system 229 without the second pulley 226 viewed from line 70-70 in FIG. 68. FIG. 70 illustrates that the gear 230 (e.g., ring gear) can have 36 teeth, for example, with an angle 234 between each tooth. The angle 234 can be, for example, 360 degrees divided by the number of teeth 230t. FIG. 70 illustrates, for example, that the gear 230 can have 36 teeth 230t such that the angle can be 10 degrees. The gear 230 and the catch 232 shown in FIG. 70 can be the second gear and the second catch. FIG. 70 illustrates that the catch 232a can have one, two, or three arms 232a with teeth (e.g., one or two teeth) that can releasably engage with the teeth 230t.

As another example, the ratchet system 229 may only have the gear 230 and the catch 232 shown in FIG. 69 or only the gear 230 and the catch 232 shown in FIG. 70.

FIG. 71 illustrates that the gear can have external teeth 230t that extend radially outward.

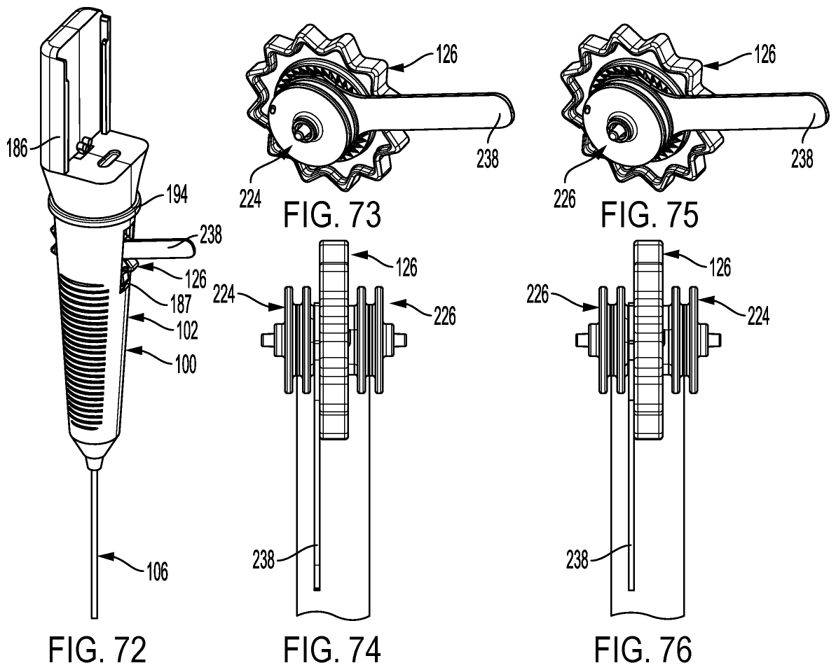

FIG. 72 illustrates that the device can have a tensioning system 236. The tensioning system 236 can including for example, the connector 233 (e.g., see FIG. 68) and a tensioner 238. The device 100 can have, for example, two connectors 233. One of the connectors 233 can be engaged to the first pulley 224, and the other of the connectors 233 (e.g., the one shown in FIG. 68) can be engaged to the second pulley 226. The tensioner 238 can be, for example, a wrench. FIG. 72 illustrates that the tensioner 238 can be removably attached to the connector 233 that is engaged with the first pulley 224 and then rotated to set the tension in the first actuator 112a or the actuator first end 112fe. FIG. 72 illustrates that the tensioner 238 can be removably attached to the connector 233 that is engaged with the second pulley 226 and then rotated to set the tension in the first actuator 112a or the actuator first end 112fe. The device 100 can be assembled such that the actuators 112 (e.g., the first and second actuators 112a, 112b or the actuator first and second ends 112fe, 112se) can be wrapped around the pulleys 224 and 226 but not in tension. To tension the actuators 112, the tensioner 138 can be removably engaged with the connectors 233 and then rotated.

For example, to set the tension in the first actuator 112a or the actuator first end 112fe, FIGS. 73 and 74 illustrate that the tensioner 138 can be engaged with the connector 233 that is connected to the first pulley 224 and then rotated in the first direction 188a to ratchet the first pulley 224 until the tension in the first actuator 112a or the actuator first end 112fe is 0.10 lbF to 1.05 lbF, including every 0.05 lbF increment within this range (e.g., 0.10 lbF, 0.50 lbF, 1.05 lbF). Once the desired tension is set, the tensioner 138 can be disengaged from the connector 233 and removed from the device 100.

As another example, to set the tension in the second actuator 112b or the actuator second end 112se, FIGS. 75 and 76 illustrate the tensioner 138 can be engaged with the connector 233 that is connected to the second pulley 226 and then rotated in the second direction 188b to ratchet the second pulley 226 until the tension in the second actuator 112b or the actuator second end 112se is 0.10 lbF to 1.05 lbF, including every 0.05 lbF increment within this range (e.g., 0.10 lbF, 0.50 lbF, 1.05 lbF). Once the desired tension is set, the tensioner 138 can be disengaged from the connector 233 and removed from the device 100.

The tensioner 138 can be used without having to use cable tensioners. The deflection of the tip 108 and the control of the flexion of the tip 108 is very dependent on the tension of the actuators 112, so being able to tune it without having to use cable tensioners or other parts can be advantageous since the tension can be set after the device is assembled, for example, before use or during use of the device 100.

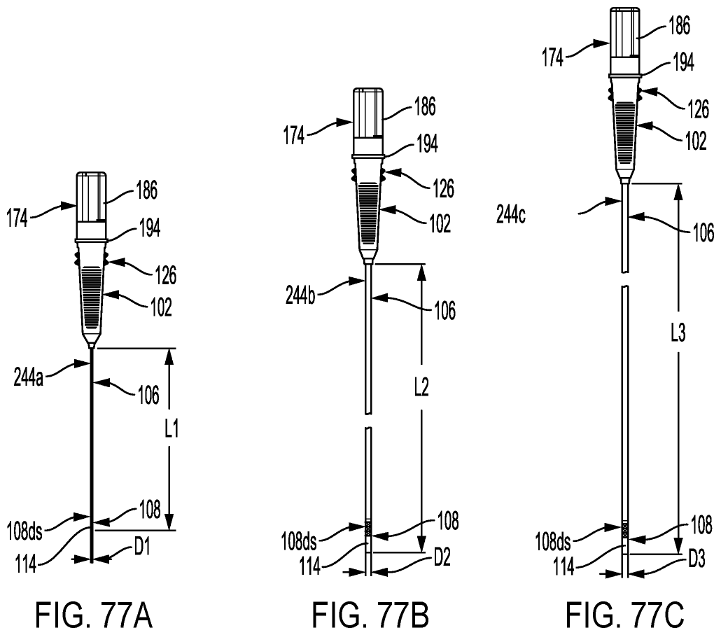

The device 100 can have different sizes 244. For example, FIGS. 77a-77c illustrate that the device 100 can have a first size 244a (e.g., a small size), a second size 244b (e.g., a medium size), and a third size 244c (e.g., a large size), respectively. FIGS. 77a-77c illustrate that the handle 102 can be the same for the different sizes 244. FIGS. 77a-77c illustrate that the body 106 can be modular, whereby bodies 106 having different lengths L and/or diameters D can be removably attached to the handle 102. FIGS. 77a-77c illustrate that the body 106 can include the tip 108 (e.g., the deflectable section 108ds and the camera 114). The different sizes can be used, for example, for different target sites.

FIG. 77a illustrates that for the first size 244a, the body 106 and the tip 108 (e.g., the deflectable section 108ds and the camera 114) can have a first length L1 (e.g., 400.00 mm) and a first diameter D1 (e.g., 3.25 mm) As shown in FIG. 77a, the first length L1 can be measured from the distal terminal end of the handle 102 to the distal terminal end of the camera 114, and the first diameter D1 can be an outer diameter of the tube 106 and the tip 108 (e.g., the deflectable section 108ds and the camera 114).

FIG. 77b illustrates that for the first size 244b, the body 106 and the tip 108 (e.g., the deflectable section 108ds and the camera 114) can have a second length L2 (e.g., 1400.00 mm) and a second diameter D2 (e.g., 10 mm) As shown in FIG. 77b, the second length L2 can be measured from the distal terminal end of the handle 102 to the distal terminal end of the camera 114, and the second diameter D2 can be an outer diameter of the tube 106 and the tip 108 (e.g., the deflectable section 108ds and the camera 114).

FIG. 77c illustrates that for the third size 244c, the body 106 and the tip 108 (e.g., the deflectable section 108ds and the camera 114) can have a second length L3 (e.g., 2000.00 mm) and a third diameter D3 (e.g., 10 mm) As shown in FIG. 77c, the third length L3 can be measured from the distal terminal end of the handle 102 to the distal terminal end of the camera 114, and the third diameter D3 can be an outer diameter of the tube 106 and the tip 108 (e.g., the deflectable section 108ds and the camera 114).

As another example, FIGS. 77a-77c illustrate three separate devices 100, where FIG. 77a illustrates the device 100 having the first size 244a, where FIG. 77b illustrates the device 100 having the second size 244b, and where FIG. 77c illustrates the device 100 having the third size 244c. FIGS. 77a-77c illustrate that the device 100 can be provided in different sizes, for example, in sizes 244a, 244b, and 244c (e.g., small, medium, and large). In some variations, the body 106 may be fixedly attached to the handle 102, such that different sizes can be provided for different target sites.

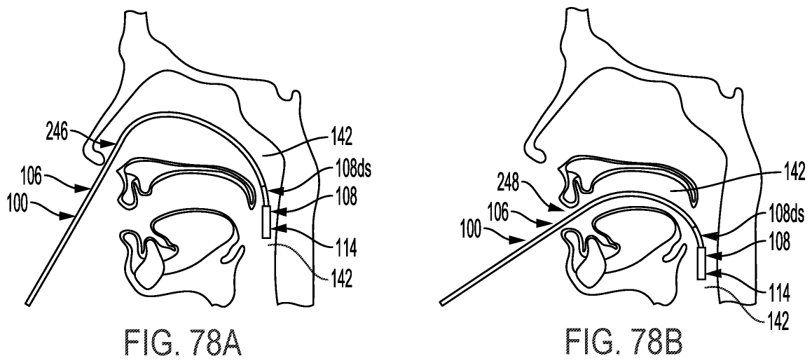

FIG. 78a illustrates the device 100 can be inserted through the nasal cavity 246, for example, to the position shown.

FIG. 78b illustrates the device 100 can be inserted through the mouth 248, for example, to the position shown.

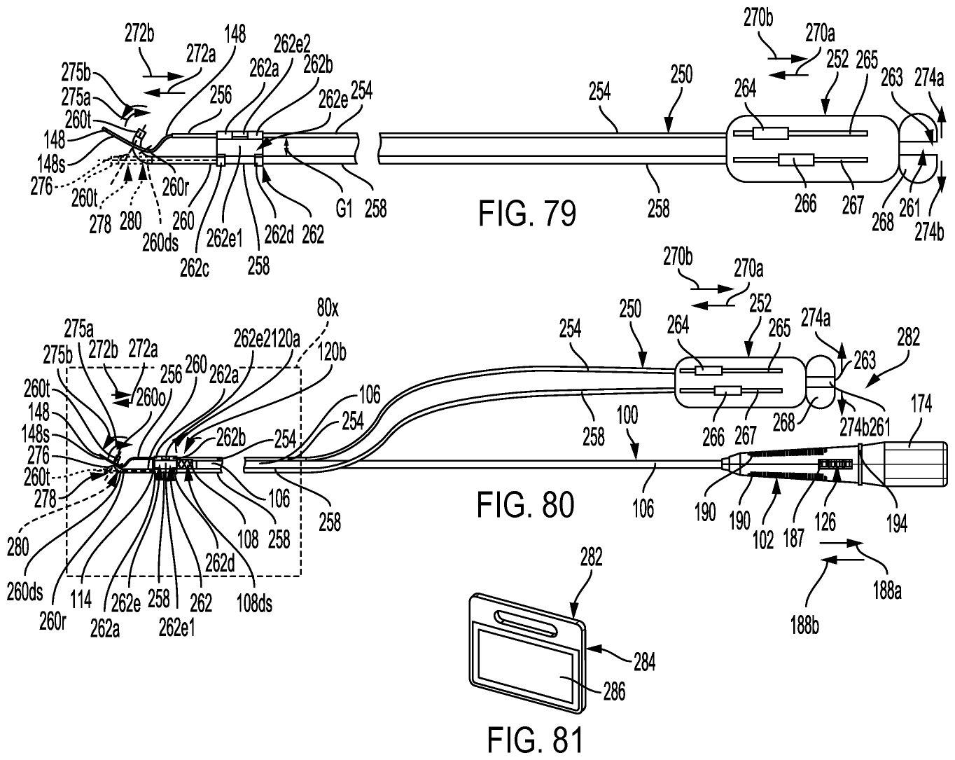

FIG. 79 illustrates a variation of a device 250 that can be inserted into body cavities, for example, into lumens of anatomical structures of a person. The device 250 can be removably attachable to the device 100. The device 250 can have a handle 252, a tube 254, a connector 256, the stabilizer 148, a tube 258, a tube 260, and a connector 262. FIG. 79 illustrates that the tube 254 and the tube 258 can be connected to each other via the connector 262. The connector 262 can removably connect the device 250 to the device 100. For example, the connector 262 can be removably connectable to the body 106 and/or the tip 108 (e.g., to the deflectable section 108ds and/or the camera 104) of the device 100. The connector 262 can be, for example, a mount that can be removably attached to the device 100 (e.g., to the body 106 and/or to the tip 108). The tube 254 can be, for example, a variety of different materials, including polymers such as Polyethylene terephthalate glycol (PETG), Teflon, metals (e.g., Nitinol, stainless steel). The tube 254 can be a bi-directional torque transmitting member such as a Helical Hollow Strand (HHS) cables. The tube 254 can be a bi-woven stainless steel cables which can allow 1:1 torque transmission. The tube 254 can be, for example, the torque transmitter 110. The tube 254 can function as a torque transmitter 110. The tube 254 can be a catheter (e.g., an HHS cable). The tube 258 can be made of variety of materials, the important characteristic of which includes lubricity as well as collapsibility (to reduce the overall outer diameter while traversing through narrow anatomy, including, for example, a polytetrafluoroethylene (PTFE) lining (e.g., inner lining), Teflon, a thermoplastic elastomer such as a polyether block amide (e.g., PEBAX) or similar material, stainless steel, or any combination thereof. The tube 258 can be a catheter. The tube 258 can be, for example, a braided or coiled tubing or catheter. The stabilizer 148 can be expandable and contractable. For example, the stabilizer 148 can be expanded by advancing the stabilizer 148 out of the tube 254 (e.g., via the control 264), and the stabilizer 148 can be contracted by retracting the stabilizer 148 into the tube 254 (e.g., via the control 264).

FIG. 79 illustrates that the handle 252 can have a control 264, a control 266, and a control 268. The control 264 can deploy the stabilizer 148. The control 264 can, for example, advance and retract the stabilizer 148 from the tube 254. The control 264 can, for example, advance and retract the connector 256 from the tube 254. The control 266 can deploy the tube 260. The control 266 can, for example, advance and retract the tube 260 from the tube 258. The control 268 can articulate the distal end of the of the tube 260, for example, from a less curved configuration (e.g., from a straight configuration) to a more curved configuration (e.g., to a curved configuration), and from a more curved configuration (e.g., from a curved configuration) to a less curved configuration (e.g., to a straight configuration).

The control 264 can be, for example, a slide, a knob, a wheel, or any combination thereof. FIG. 79 illustrates, for example, that the control 264 can be a slide that can be movable (e.g., translatable) in a track 265. The track 265 can be, for example, a recess or a slot in the handle 252. The control 264 can be moved (e.g., translated) in a first direction 270a to advance the stabilizer 148 in a first direction 272a out of the tube 254. The control 264 can be moved (e.g., translated) in a second direction 270b to retract the stabilizer 148 in a second direction 272b into the tube 254. The second direction 270b can be opposite the first direction 270a. The second direction 272b can be opposite the first direction 272a. The control 264 can be moved (e.g., translated) in the first direction 270a to advance the connector 256 in the first direction 272a out of the tube 254. The control 264 can be moved (e.g., translated) in the second direction 270b to retract the connector 256 in the second direction 272b into the tube 254. FIG. 79 illustrates that the tube 254 can extend through the connector 262 to a distal terminal end of the connector 262 such that the control 264 can be moved (e.g., translated) in the first direction 270a to advance the stabilizer 148 in the first direction 272a out of the tube 254 and out of the connector 262, such that the control 264 can be moved (e.g., translated) in the second direction 270b to retract the stabilizer 148 in the second direction 272b into the tube 254 and into the connector 262, such that the control 264 can be moved (e.g., translated) in the first direction 270a to advance the connector 256 in the first direction 272a out of the tube 254 and out of the connector 262, such that the control 264 can be moved (e.g., translated) in the second direction 270b to retract the connector 256 in the second direction 272b into the tube 254 and into the connector 262, or any combination thereof. FIG. 79 illustrates the stabilizer 148 in a fully advanced configuration (e.g., in a fully deployed configuration). As another example, FIG. 79 illustrates the stabilizer 148 in a partially advanced configuration (e.g., in a partially deployed configuration). A distal end of the connector 256 can be connected to the stabilizer 148 (e.g., to a proximal end of the stabilizer 148), and a proximal end of the connector 256 can be connected to the control 264. The connector 256 can be, for example, a cable or a rod. FIG. 79 illustrates that the connector 256 can be a cable.

The control 266 can be, for example, a slide, a knob, a wheel, or any combination thereof. FIG. 79 illustrates, for example, that the control 266 can be a slide that can be movable (e.g., translatable) in a track 267. The track 267 can be, for example, a recess or a slot in the handle 252. The control 266 can be moved (e.g., translated) in the first direction 270a to advance the tube 260 in the first direction 272a out of the tube 258. The control 266 can be moved (e.g., translated) in the second direction 270b to retract the tube 260 in the second direction 272b into the tube 258. FIG. 79 illustrates that the tube 258 can extend through the connector 262 to a distal terminal end of the connector 262 such that the control 266 can be moved (e.g., translated) in the first direction 270a to advance the tube 260 in the first direction 272a out of the tube 254 and out of the connector 262, and such that the control 266 can be moved (e.g., translated) in the second direction 270b to retract the tube 260 in the second direction 272b into the tube 254 and into the connector 262. FIG. 79 illustrates the tube 260 in a partially advanced configuration (e.g., in a partially deployed configuration). As another example, FIG. 79 illustrates the tube 260 in a fully advanced configuration (e.g., in a fully deployed configuration). A proximal end of the tube 260 can be connected to the control 266. For example, the proximal end of the tube 260 can be directed connected tot eh control 266 or can be connected via a rod or a cable.

The control 268 can be, for example, a slide, a knob, a wheel, or any combination thereof. FIG. 79 illustrates, for example, that the control 268 can be a rotatable knob. The tube 260 can have a deflectable section 260ds. The tube 260 can be, for example, a deployable catheter. The tube 260 can be, for example, a deployable catheter having the deflectable section 260ds. The tube 258 can be, for example, a catheter housing. FIG. 79 illustrates, for example, that the deflectable section 260ds can be the distal end of the tube 260. The control 268 can be rotated in a first direction 274a to deflect the tube 260 (e.g., to bend the deflectable section 260ds), and the control 268 can be rotated in a second direction 274b to straighten the tube 260 (e.g., to straighten the deflectable section 260ds). The second direction 274b can be opposite the first direction 274a. The control 268 can be rotated in the first direction 274a to move (e.g., rotate) a distal end of the tube 260 (e.g., the deflectable section 260ds) in a first direction 275a, and the control 268 can be rotated in a second direction 274b to move (e.g., rotate) the distal end of the tube 260 (e.g., the deflectable section 260ds) in a second direction 275b. The second direction 275b can be opposite the first direction 275a.

For example, the control 268 can be rotated in the first direction 274a to articulate the deflectable section 260ds, for example, from a non-deflected configuration to a deflected configuration and/or from a first deflected configuration to a second deflected configuration. For example, the control 268 can be rotated in the first direction 274a to articulate the deflectable section 260ds from a straight configuration (e.g., from the straight configuration shown in dashed lines in FIG. 79) to a deflected configuration (e.g., to the curved configuration shown in FIG. 79). As another example, the control 268 can be rotated in the second direction 274b to articulate the deflectable section 260ds, for example, from the deflected configuration to the non-deflected configuration and/or from the second deflected configuration to the first deflected configuration. For example, the control 268 can be rotated in the second direction 274b to articulate the deflectable section 260ds from the deflected configuration (e.g., from the curved configuration shown in FIG. 79) to a straight configuration (e.g., to the straight configuration shown in dashed lines in FIG. 79). For example, the control 268 can be rotated in the first direction 274a to decrease the radius of curvature 260r of the deflectable section 260ds, and the control 268 can be rotated in the second direction 274b to increase the radius of curvature 260r of the deflectable section 260ds. The radius of curvature 260r can be, for example, 100.0 mm to 2 mm, or more narrowly, 50 mm to 2 mm, or more narrowly still, 25 mm to 2 mm, including every 0.5 mm increment within these ranges (e.g., 100.0 mm, 50.0 mm, 25.0 mm, 10.0 mm, 5.0 mm, 2.0 mm) For example, FIG. 79 illustrates that when the deflectable section 260ds is in a deflected configuration (e.g., a fully deflected configuration), the radius of curvature 260r can be 5.0 mm or 10.0 mm. The distal end of the tube 260 can have a tip 260t. The tip 260t can be the distal end of the deflectable section 260ds. The tip 260t can be part of the deflectable section 260ds or the tip 260t can extend from the deflectable section 260ds. For example, FIG. 79 illustrates that the tip 260t can extend from the deflectable section 260ds. FIG. 79 illustrates that the deflectable section 260ds can be articulated so that an axis A2 of the tip 260t (e.g., a center longitudinal axis of the tip 260t) can be at an angle 276 with an axis A3 of the tube 260 proximal the deflectable section 260ds (e.g., a center longitudinal axis of the tube 260 proximal the deflectable section 260ds. The control 268 can be rotated in the first direction 274a to increase the angle 276, and the control 268 can be rotated in the second direction 274b to decrease the angle 276. The angle 276 can be, for example, 0 degrees to 180 degrees, or more narrowly, 0 degrees to 170 degrees, or more narrowly still, 0 degrees to 150 degrees, or more narrowly still, 0 degrees to 90 degrees, including every 1 degree increment within these ranges (e.g., 0 degrees, 15 degrees, 30 degrees, 45 degrees, 60 degrees, 75 degrees, 90 degrees, 105 degrees, 120 degrees, 135 degrees, 150 degrees, 165 degrees, 170 degrees, 180 degrees). For example, FIG. 79 illustrates that the angle 276 can be 105 degrees. The control 268 can be rotated in the first direction 274a to deflect (e.g., bend) the deflectable section 260ds to move (e.g., rotate) the tip 260t in the first direction 275a, for example, through the angle 276, and the control 268 can be rotated in the second direction 274b to deflect (e.g., straighten) the deflectable section 260ds to move (e.g., rotate) the tip 260t in the second direction 275b, for example, through the angle 276).

FIG. 79 illustrates the deflectable section 260ds can have a deflected configuration 280. The deflected configuration 280 can be a partially deflected configuration of the deflectable section 260ds or can be a fully deflected configuration of the deflectable section 260ds. For example, FIG. 79 illustrates that the deflected configuration 280 can be a fully deflected configuration of the deflectable section 260ds, whereby the deflectable section 260ds can have a partially deflected configuration, for example, anywhere between the non-deflected configuration and the fully deflected configuration. FIG. 79 illustrates that the deflectable section 260ds can have a non-deflected configuration 278. When the deflectable section 260ds has the non-deflected configuration 278, the deflectable section 260ds can be straight or can be less curved than when in the deflected configuration 280. For example, FIG. 79 illustrates that when the deflectable section 260ds has the non-deflected configuration 278, the deflectable section 260ds can be straight. The control 268 can be connected to the deflectable section 260ds (e.g., to a distal end of the deflectable section 260ds) via an articulator. The articulator can be, for example, a wire, a rod, or a cable. FIG. 79 illustrates, for example, that the articulator can be a wire. When the control 268 is rotated in the first direction 274a, the tension in the articulator can be increased, and when the control 268 is rotated in the second direction 274b, the tension in the articulator can be decreased.

FIG. 79 illustrates that the connector 262 can be connected to the tube 254 and/or to the tube 258. The connector 262 can be fixedly attached to the tube 254 and the tube 258. The connector 262 can be removably attached to the tube 254 and the tube 258. When the connector 262 is attached to the tube 254 and the tube 258, the connector 262 can be slidable along the tube 254 and the tube 258 in the first direction 272a and the second direction 272b. As another example, when the connector 262 is attached to the tube 254 and the tube 258, the connector 262 may not be slidable along the tube 254 and the tube 258. For example, when the connector 262 is attached to the tube 254 and the tube 258, the connector 262 can have a fixed position on the tube 254 and the tube 258. FIG. 79 illustrates, for example, that when the connector 262 is attached to the tube 254 and to the tube 258, the connector 262 may not be slidable along the tube 254 and the tube 258. FIG. 79 illustrates that the connector 262 can space the tube 254 and the tube 258 apart by a gap G1. FIG. 79 illustrates, for example, that when the connector 262 is attached to the device 250, the tube 254 and the tube 258 can be separated by a gap G1. The gap G1 can be, for example, 1 mm to 30 mm, or more narrowly, 1 mm to 20 mm, including every 1 mm increment within these ranges (e.g., 1 mm, 5 mm, 10 mm, 12 mm, 20 mm, 30 mm) For example, FIG. 79 illustrates that the gap G1 can be 3.50 mm or 17.50 mm. The device 100 (e.g., the body 106, the deflectable section 108ds, and/or the camera 114) can be removably positioned in the gap G1.

FIG. 79 illustrates that the connector 262 can be a mount that can removably connect the device 100 and the device 250 together. FIG. 79 illustrates that the connector 262 can be a mount that can removably secure the device 100 to the device 250. The device 100 can be removably connected to the connector 262. The device 250 can be removably connected to the device 100, for example, via the connector 262. The connector 262 can be, for example, silicone. The connector 262 can releasably grip a surface of the device 100, for example, a surface of the body 106, the tip 108, and/or the camera 114. The connector 262 can be, for example, a sheath (e.g., a silicone sheath) that can be attached (e.g., wrapped) around the device 100.

FIG. 79 illustrates that the connector 262 can have a mount 262a, a mount 262b, a mount 262c, a mount 262d, and a mount 262e, or any combination thereof. The mount 262a and/or the mount 262b can attach the connector 262 to the tube 254. The mount 262c and/or the mount 262d can attach the connector 262 to the tube 254. The mount 262e can attach the connector 262 to the device 100, for example, to the tube 106, to the tip 108, to the camera 114, or any combination thereof. The mount 262e can, for example, releasably attach the connector 262 to the to the tube 106, to the deflectable section 108ds, to the camera 114, or any combination thereof. For example, FIG. 79 illustrates that the mount 262e can be releasably attachable to the body 106, to the deflectable section 108ds, to the camera 114, or to any combination thereof. FIG. 79 illustrates, for example, that the mount 262e can have a body 262e1 and a connector 262e2. The body 262e1 can be removably attached (e.g., removably wrapped) to the device 100, for example, to the body 106, to the deflectable section 108ds, to the camera 114, or to any combination thereof, and the connector 262e2 can be removably attached (e.g., removably clipped) to the tube 254. For example, FIG. 79 illustrates that the body 262e1 can be releasably wrapped around the device 100 (e.g., partially or completely around the body 106, partially or completely around the deflectable section 108ds, and/or partially or completely around the camera 114) and the connector 262e2 can be removably attached to the tube 254. The connector 262e1 can be flexible such that it can conform to the outside shape of the device 100. For example, the connector 262e1 can be flexible such that it can be partially or completely wrapped around the body 106, the deflectable section 108ds, the camera 114, or any combination thereof. The connector 262e2 can be, for example, a clip such that the connector 262e2 can be removably clipped to the tube 254. The mount 262a, the mount 262b, the mount 262c, the mount 262d, and the mount 262e can also be referred to as the first mount 262a, the second mount 262b, the third mount 262c, the fourth mount 262d, and the fifth mount 262e, or any combination thereof.

The tube 258 can have a thin wall, for example, a 0.1 mm to 0.5 mm thickness so that the diameter of the tube 258 can collapse. The tube 260 may not be advanced in the tube 258 until the tip 108 is in position at the target site 144 so that the tube 258 can compress (e.g., so that the diameter of the tube 258 can decrease) to traverse through narrow anatomy. The connector 262c and the connector 262d can be flexible to allow the collapsing of the tube 258 upon contact with narrowing anatomy, whereby the connector 262c and/or the connector 262d can collapse (e.g., the width of a channel through the connector 262c and the width of a channel through the connector 262d can collapse).

FIG. 79 illustrates that the stabilizer 148 can extend from the connector 256, and that the connector 256 can extend from the tube 254. FIG. 79 illustrates that the stabilizer 148 can extend from a distal terminal end of the connector 256. FIG. 79 illustrates that the stabilizer 148 can be connected to the connector 256. For example, FIG. 79 illustrates that a proximal end of the stabilizer 148 can be connected to a distal end of the connector 256. A proximal terminal end of the stabilizer 148 can be connected to a distal terminal end of the connector 256. As another example, the stabilizer 148 can extend from the tube 254. For example, the device 250 may not have the connector 256.

FIG. 79 illustrates that when the stabilizer 148 is in a deployed configuration (e.g., in a partially deployed configuration or in a fully deployed configuration), the tube 260 can be movable into and out of a space 148s defined by the stabilizer 148. For example, FIG. 79 illustrates that the deflectable section 260ds and the tip 260t can be movable into and out of the space 148s. The space 148s can be, for example, an opening through the stabilizer. The space 148s can be, for example, a gap between two opposite sides of the stabilizer 148. FIG. 79 illustrates, for example, that when the stabilizer 148 is in a deployed configuration (e.g., in a partially deployed configuration or in a fully deployed configuration), the deflectable section 260ds can be in the space 148s. For example, FIG. 79 illustrates that the control 268 can be rotated in the first direction 274a to move the tube 260 (e.g., the deflectable section 260ds and/or the tip 260t) into the space 148s, and that the control 268 can be rotated in the second direction 274b to move the tube 260 (e.g., the deflectable section 260ds and/or the tip 260t) out of the space 148s. FIG. 79 illustrates, for example, that when the stabilizer 148 is in a deployed configuration (e.g., in a partially deployed configuration or in a fully deployed configuration), the tube 260 (e.g., the deflectable section 260ds and/or the tip 260t) can extend through the space 148s. For example, FIG. 79 illustrates that when the stabilizer 148 is in a fully deployed configuration (e.g., in a fully advanced position), when the tube 260 is in a deployed configuration (e.g., in a partially advanced position or in a fully advanced position), and when the tube 260 is in a deflected configuration (e.g., the deflected configuration 280), the tube 260 (e.g., the deflectable section 260ds and/or the tip 260t) can be inside the stabilizer 148 (e.g., in the space 148s of the stabilizer 148). As another example, FIG. 79 illustrates that when the stabilizer 148 is in a fully deployed configuration (e.g., in a fully advanced position), when the tube 260 is in a deployed configuration (e.g., in a partially advanced position or in a fully advanced position), and when the tube 260 is in a non-deflected configuration (e.g., the non-deflected configuration 278), the tube 260 (e.g., the deflectable section 260ds and the tip 260t) can be outside the stabilizer 148 (e.g., outside the space 148s of the stabilizer 148).

FIG. 79 illustrates that when the stabilizer 148 is in a deployed configuration (e.g., in a partially deployed configuration or in a fully deployed configuration), the tube 260 can be movable from a first side of the stabilizer 148 to a second side of the stabilizer 148, and that the tube 260 can be movable from the second side of the stabilizer 148 to the first side of the stabilizer 148. The first side of the stabilizer 148 can be, for example, on a first side of the space 148s, and the second side of the stabilizer 148 can be, for example, on a second side of the space 148s. The second side of the stabilizer 148 can be opposite the first side of the stabilizer 148. For example, FIG. 79 illustrates that the tube 260 (e.g., the deflectable section 260ds and the tip 260t) can be movable from a first side of the stabilizer 148 (e.g., from a first side of the space 148s) to a second side of the stabilizer 148 (e.g., to a second side of the space 148s). The first side of the space 148s can be below the space 148s, and the second side of the space 148s can be above the space 148, or vice versa. FIG. 79 illustrates that when the stabilizer 148 is in a deployed configuration (e.g., in a partially deployed configuration or in a fully deployed configuration) and when the deflectable section 260ds is in a deflected configuration (e.g., in a partially deflected configuration or in a fully deflected configuration), the deflectable section 260ds can be on a first side of the stabilizer 148 (e.g., on a first side of the space 148s) and on a second side of the stabilizer 148 (e.g., on a second side of the space 148s). FIG. 79 illustrates that when the stabilizer 148 is in a deployed configuration (e.g., in a partially deployed configuration or in a fully deployed configuration) and when the deflectable section 260ds is in a non-deflected configuration (e.g., in a straight configuration), the deflectable section 260ds can be on a first side of the stabilizer 148 (e.g., on a first side of the space 148s) but not on a second side of the stabilizer 148 (e.g., but not on a second side of the space 148s). For example, FIG. 79 illustrates that the control 268 can be rotated in the first direction 274a to move the tube 260 (e.g., the deflectable section 260ds and/or the tip 260t) from a first side of the stabilizer 148 to the second side of the stabilizer 148, and that the control 268 can be rotated in the second direction 274b to move the tube 260 (e.g., the deflectable section 260ds and/or the tip 260t) from the second side of the stabilizer 148 to the first side of the stabilizer 148.

The stabilizer 148 can be advanced and retracted independently of the tube 260 and/or independently of the position of the tube 260. For example, when the tube 260 is in a fully retracted configuration, the stabilizer 148 can be advanced and retracted (e.g., using the control 264), when the tube 260 is in a partially advanced configuration, the stabilizer 148 can be advanced and retracted (e.g., using the control 264), when the tube 260 is in a fully advanced configuration, the stabilizer 148 can be advanced and retracted (e.g., using the control 264), or any combination thereof.

The tube 260 can be advanced and retracted independently of the stabilizer 148 and/or independently of the position of the stabilizer 148. For example, when the stabilizer 148 is in a fully retracted configuration, the tube 260 can be advanced and retracted (e.g., using the control 266), when the stabilizer 148 is in a partially advanced configuration, the tube 260 can be advanced and retracted (e.g., using the control 266), when the stabilizer 148 is in a fully advanced configuration, the tube 260 can be advanced and retracted (e.g., using the control 266), or any combination thereof.

The tube 260 can be articulated independently of the stabilizer 148 and/or independently of the position of the stabilizer 148. For example, when the stabilizer 148 is in a fully retracted configuration, the tube 260 can be articulated (e.g., using the control 268), when the stabilizer 148 is in a partially advanced configuration, the tube 260 can be articulated (e.g., using the control 268), when the stabilizer 148 is in a fully advanced configuration, the tube 260 can be articulated (e.g., using the control 268), or any combination thereof.

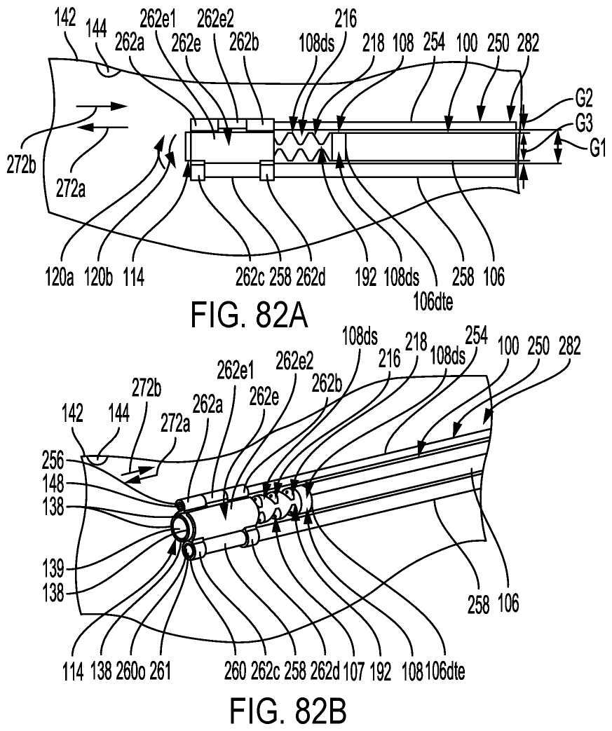

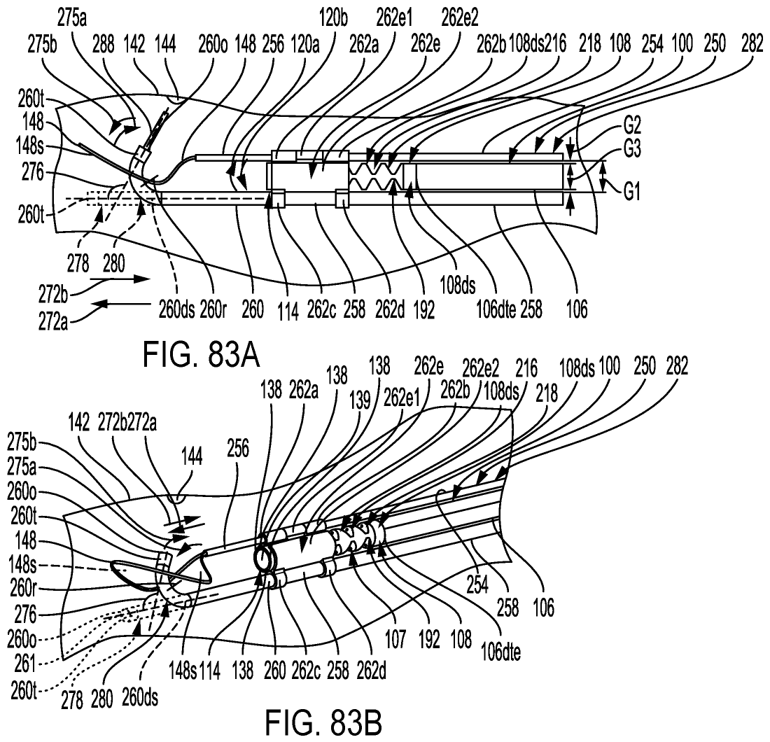

FIG. 79 illustrates that the tube 260 can have a lumen 261 (see e.g., the lumen 261 in FIGS. 82b, 83b, and 84b) that can be a working channel that other tools or devices can be advanced and retracted in when the tube 260. The other tools or devices can be advanced and retracted in the tube 260 when the tube 260 is in a fully retracted configuration, when the tube 260 is in an advanced configuration and the deflectable section 260ds has a non-deflected configuration, when the tube 260 is in an advanced configuration and the deflectable section 260ds has a deflected configuration, or any combination thereof. The other tools or devices (e.g., the object 288 in FIG. 83a) can be, for example, a guidewire, a visualization device (e.g., a camera), a tissue harvester (e.g., to collect a tissue biopsy sample), a tissue ablater (e.g., a tissue ablation device), a needle, an electrosurgical device, a retrieval basket, an implant (e.g., a stent), a biological compatible material (e.g., absorbable PLGA and/or metal), a closure device (e.g., a suture device and/or a stapling device), or any combination thereof.

FIG. 79 illustrates that when the stabilizer 148 is in a fully deployed configuration (e.g., in a fully advanced position), when the tube 260 is in a partially deployed configuration (e.g., in a partially advanced position), and when the tube 260 is in a fully deflected configuration (e.g., see the deflected configuration 280), the stabilizer 148 and the tube 260 can have the arrangement of features shown, including the relative positions between these features, whereby, for example, the distal terminal end of the stabilizer 148 can be the distal terminal end of the device 250 and the tube 260 can extend through the stabilizer 148 (e.g., between two sides of the stabilizer 148, for example, through the space 148s).

FIG. 79 illustrates that when the stabilizer 148 is in a fully deployed configuration (e.g., in a fully advanced position), when the tube 260 is in a partially deployed configuration (e.g., in a partially advanced position), and when the tube 260 is in a non-deflected configuration (e.g., see the non-deflected configuration 278), the stabilizer 148 and the tube 260 can have the arrangement of features shown, including the relative positions between these features, whereby, for example, the distal terminal end of the stabilizer 148 can be the distal terminal end of the device 250 and the tube 260 may not extend through the stabilizer 148 (e.g., between two sides of the stabilizer 148, for example, through the space 148s).

When the stabilizer 148 is in a fully deployed configuration (e.g., in a fully advanced position), when the tube 260 is in a fully deployed configuration (e.g., in a fully advanced position), and when the tube 260 is in a non-deflected configuration (e.g., see the non-deflected configuration 278), the stabilizer 148 can be the distal terminal end of the device 250 (e.g., by 1 mm to 20 mm, including every 1 mm increment within this range), the tube 260 can be the distal terminal end of the device 250 (e.g., by 1 mm to 20 mm, including every 1 mm increment within this range), or the stabilizer 148 and the tube 260 be the distal terminal end of the device 250.

The stabilizer 148 and the tube 260 can be moved relative to each other in the first and second directions 272a, 272b

When the tube 260 is in a deflected configuration (e.g., in the deflected configuration 280), the tube 260 can be moved relative to the stabilizer 148 in the first and second directions 272a, 272b. For example, FIG. 79 illustrates that when the stabilizer 148 is in a deployed configuration (e.g., in a partially advanced position or in a fully advanced position) and when the tube 260 is in a deflected configuration (e.g., see the deflected configuration 280), the deflectable section 260ds and the tip 260t can be movable (e.g., translatable) in the first direction 272a (e.g., via the control 266) and can be movable (e.g., translatable) in the second direction 272b (e.g., via the control 266), for example, between a first longitudinal terminal end of the stabilizer 148 and a second longitudinal end of the stabilizer 148. The second longitudinal end of the stabilizer 148 can be opposite the first longitudinal end of the stabilizer 148. For example, the first longitudinal end of the stabilizer 148 can be a proximal end (e.g., a proximal terminal end) of the stabilizer 148, and the second longitudinal end of the stabilizer 148 can be a distal end (e.g., a distal terminal end) of the stabilizer 148). When the tube 260 is in a deflected configuration (e.g., the deflected configuration 280), the deflectable section 260ds and the tip 260t can be movable (e.g., translatable) in the first and second directions 272a, 272b between the first and second longitudinal ends of the stabilizer 148 (e.g., between a proximal end and a distal end of the space 148s). For example, FIG. 79 illustrates that the tube 260 can be in a deflected configuration (e.g., the deflected configuration 280) between the proximal terminal end of the space 148s and the distal terminal end of the space 148s.

When the tube 260 is in a deflected configuration (e.g., in the deflected configuration 280), the stabilizer can be moved relative to the tube 260 in the first and second directions 272a, 272b. For example, FIG. 79 illustrates that when the tube 260 is in a deflected configuration (e.g., in a partially deflected configuration or in a fully deflected configuration) and when the stabilizer 148 is in a deployed configuration (e.g., in a partially advanced position or in a fully advanced position), the stabilizer 148 can be movable (e.g., translatable) in the first direction 272a (e.g., via the control 264) and can be movable (e.g., translatable) in the second direction 272b (e.g., via the control 264) such that the position of the tube 260 (e.g., the deflectable section 260ds and/or the tip 260t) in the space 148s can be adjusted. When the tube 260 is in a deflected configuration (e.g., the deflected configuration 280), the stabilizer 148 can be movable (e.g., translatable) in the first and second directions 272a, 272b while keeping the tube 260 in the space 148s.

The stabilizer 148 may or may not be rotatable. For example, FIG. 79 illustrates that the stabilizer 148 may not be rotatable. As another example, however, the stabilizer 148 can be rotatable, for example, in the first and second directions 274a, 274b, via a rotatable control on the handle 252 (e.g., like the control 268). In such cases, the stabilizer 148 can be rotated about the axis (e.g., center longitudinal axis) of the connector 256 and/or about the axis (e.g., center longitudinal axis) of the tube 254). Rotating the stabilizer 148 can, for example, articulate (e.g., rotate) the distal end of the tube 260 (e.g., the deflectable section 260ds and the tip 260t) in the first and second directions 274a, 274b, respectively.

FIG. 80 illustrates the device 100 (e.g., the device 100 of FIG. 44) removably attached to the device 250, for example, via the connector 262. The body 262e1 can be removably attached to (e.g., removably wrapped around) the device 100, for example, to the body 106, to the deflectable section 108ds, to the camera 114, or to any combination thereof, and the connector 262e2 can be removably attached (e.g., removably clipped) to the tube 254. For example, FIG. 80 illustrates that the body 262e1 can be removably attached to (e.g., removably wrapped around) the camera 114, and that the connector 262e2 can be removably attached (e.g., removably clipped) to the tube 254. FIG. 80 illustrates, for example, that the device 250 can be attached to the distal end of the device 100 (e.g., to the camera 114). As another example, the device 250 can be attached to the tube 106 proximal the deflectable section 108ds.

FIG. 80 illustrates that when the device 100 and the device 250 are attached to each other, for example, via the connector 262, the device 100 can be operated independently of the device 250, and the device 250 can be operated independently of the device 100. The device 100 can be operated, for example, via the controls on handle 102 (e.g., with the control 126) and/or the controls on the module 174), and the device 250 can be operated, for example, via the controls on the handle 252 (e.g., the controls 264, 266, and 268).

FIG. 80 illustrates that when the device 100 is attached to the device 250, for example, via the connector 262, the device 100 can be in the gap G1. For example, FIG. 80 illustrates that when the device 100 is attached to the device 250, the body 106, the deflectable section 180ds, and the camera 114 can be in the gap G1. The handle 102 and the handle 252 may not be connected to each other, for example, as shown in FIG. 80. In such cases, the tube 254 and the tube 258 can extend away from the body 106 and vice versa. For example, FIG. 80 illustrates that a distal end of the body 106 can be in the gap G1 and that a proximal end of the body 106 can be outside of the gap G1. As another example, the handle 102 and the handle 252 can be removably connected to each other, for example, via a clip.

FIG. 80 illustrates that a system 282 can include, for example, the device 100 and the device 250.

FIG. 80 illustrates that when the device 100 is attached to the device 250, for example, via the connector 262, the deflectable section 108ds can be deflectable in the first direction 120a and in the second direction 120b. For example, FIG. 80 illustrates that when the device 100 is attached to the device 250 and the deflectable section 108ds is deflected in the first direction 120a (e.g., via the control 126), the camera 114, the stabilizer 148, the connector 256, the tube 258, the tube 260, or any combination thereof can move in the first direction 120a with the deflectable section 108ds. As another example, FIG. 80 illustrates that when the device 100 is attached to the device 250 and the deflectable section 108ds is deflected in the second direction 120b (e.g., via the control 126), the camera 114, the stabilizer 148, the connector 256, the tube 258, the tube 260, or any combination thereof can move in the second direction 120b with the deflectable section 108ds. In other words, FIG. 80 illustrates that the deflectable section 108ds can be articulated in the first and second directions 120a, 120b to articulate the camera 114, the stabilizer 148, the connector 256, the tube 258, the tube 260, or any combination thereof in the first and second directions 120a, 120b. For example, when the device 100 is attached to the device 250, for example, via the connector 262, the distal end of the device 250 can be articulated in the first direction 120a by articulating the deflectable section 108ds of the first device 100 in the first direction 120a (e.g., via the control 126). As another example, when the device 100 is attached to the device 250, for example, via the connector 262, the distal end of the device 250 can be articulated in the second direction 120b by articulating the deflectable section 108ds of the first device 100 in the second direction 120b (e.g., via the control 126) in the second direction 120b.

FIGS. 79 and 80 illustrate that the lumen 261 of the tube 260 can extend into the handle 252 and terminate at an opening 263. The opening 263 can be the proximal opening to the lumen 261 for the tube 260. Objects 288 can be inserted into the lumen 261 and withdrawn from the lumen 261 via the opening 263. The opening 263 can be anywhere on the handle 252. For example, FIG. 80 illustrates that the lumen 261 can extend through the handle 252 and the control 268, and that the opening 263 can be on a proximal end of the control 268. The opening 263 can be a port on the surface of the handle 252. For example, FIG. 80 illustrates that the opening 263 can be a port on the surface of the control 268.

FIG. 80 illustrates that when the device 100 is attached to the device 250, the device 100 (e.g., the body 106, the deflectable section 108ds, and/or the camera 114) can be inside the device 250, for example, inside the connector 262. FIG. 80 illustrates that when the device 100 is attached to the device 250, the device 100 (e.g., the body 106, the deflectable section 108ds, and/or the camera 114) can extend through the device 250, for example, through the connector 262. FIG. 80 illustrates that when the device 100 is attached to the device 250, the device 250 can be outside of the device 100. For example, FIG. 80 illustrates that when the device 100 is attached to the device 250, the tube 254, the connector 256, the stabilizer 148, the tube 258, the tube 260, the connector 262, or any combination thereof can be outside of the device 100.

FIG. 81 illustrates that the system 282 can include, for example, the device 100, the device 250, and a computer 284. The computer 284 can have a display 286. The device 100 and/or the device 250 can be in wired and/or wireless communication with the computer 284. For example, the module 174 can be in wired and/or wireless communication with the computer 284. As another example, the camera 114 can be in wired and/or wireless communication with the computer 284. During operation, images captured from the device 100 (e.g., from the camera 114) can be displayed on the display 286, for example, in real-time. The computer 284 can thereby be used to visualize the images from the camera 114. The computer 284 can be, for example, a desktop computer. The computer 284 can be, for example, a portable computer such as a tablet or a smartphone.

FIG. 82a illustrates a closeup of section 80x in FIG. 80 with the device 100 and the device 250 in a body cavity 142 at a target site 144, with the stabilizer 148 in a non-deployed configuration and with the tube 260 in a non-deployed configuration.

FIG. 82a illustrates that when the stabilizer 148 is in a non-deployed configuration, the stabilizer 148 can be inside the tube 254. FIG. 82a illustrates that when the stabilizer 148 is in a non-deployed configuration, the stabilizer 148 can be inside the tube 254 and inside the connector 262. When the stabilizer 148 is in a non-deployed configuration, the stabilizer 148 can be inside the mount 262a, the connector 262e2, the mount 262b, or any combination thereof. For example, FIG. 82a illustrates that when the stabilizer 148 is in a non-deployed configuration, the stabilizer 148 can be inside the mount 262a, the connector 262e2, and the mount 262b. For example, the stabilizer 148 can extend through a lumen in the tube 254, a lumen in the mount 262a, a lumen in the connector 262e2, a lumen in the mount 262b, or any combination thereof. FIG. 82a illustrates that when the stabilizer 148 is in a non-deployed configuration, the stabilizer 148 can be in a fully retracted position in the tube 254. When the stabilizer 148 is in a non-deployed configuration, the stabilizer 148 can be fully inside the tube 254. For example, FIG. 82a illustrates that when the stabilizer 148 is in a non-deployed configuration, the stabilizer 148 may not extend from the tube 254 and/or from the connector 262 such that the distal end of the stabilizer 148 is completely inside the tube 254 and/or inside the connector 262. As another example, when the stabilizer 148 is in a non-deployed configuration, the stabilizer 148 may extend from the tube 254 and/or from the connector 262 such that the distal end of the stabilizer 148 can be outside the tube 254 and/or the connector 262.

FIG. 82a illustrates that when the tube 260 is in a non-deployed configuration, the tube 260 can be inside the tube 258. FIG. 82a illustrates that when the tube 260 is in a non-deployed configuration, the tube 260 can be inside the tube 258 and inside the connector 262. When the tube 260 is in a non-deployed configuration, the tube 260 can be inside the mount 262c and/or the mount 262d. For example, FIG. 82a illustrates that when the tube 260 is in a non-deployed configuration, the tube 260 can be inside the mount 262c and the mount 262d. For example, the tube 260 can extend through a lumen in the tube 258, a lumen in the mount 262c and/or a lumen in the mount 262d. FIG. 82a illustrates that when the tube 260 is in a non-deployed configuration, the tube 260 can be in a fully retracted position in the tube 258. When the tube 260 is in a non-deployed configuration, the tube 260 can be fully inside the tube 258. For example, FIG. 82a illustrates that when the tube 260 is in a non-deployed configuration, the tube 260 may not extend from the tube 258 and/or from the connector 262 such that the distal end of the tube 260 is completely inside the tube 258 and/or inside the connector 262. As another example, when the tube 260 is in a non-deployed configuration, the tube 260 may extend from the tube 258 and/or from the connector 262 such that the distal end of the tube 260 can be outside the tube 258 and/or the connector 262.

FIG. 82a illustrates that the connector 262 can space the tube 254 and the device 100 apart by a gap G2. The gap G2 can be between the tube 254 and the camera 114, between the tube 254 and the deflectable section 108ds, between the tube 254 and the body 106, or any combination thereof. FIG. 82a illustrates, for example, that when the device 100 is attached to the connector 262, the tube 254 and the camera 114 can be separated by the gap G2, the tube 254 and the deflectable section 108ds can be separated by the gap G2, and the tube 254 and the body 106 can be separated by the gap G2. The gap G2 can be, for example, 0.0 mm to 10.0 mm, or more narrowly, 0.0 mm to 5.0 mm, including every 0.1 mm increment within these ranges (e.g., 0.0 mm, 1.0 mm, 2.0 mm, 5.0 mm, 10.0 mm). For example, FIG. 82a illustrates that the gap G1 can be 1.5 mm. When the gap G2 is 0.0 mm, the tube 254 can contact the device 100 when the device is connected to the connector 262.

FIG. 82a illustrates that the connector 262 can space the tube 258 and the device 100 apart by a gap G3. The gap G3 can be between the tube 258 and the camera 114, between the tube 258 and the deflectable section 108ds, between the tube 258 and the body 106, or any combination thereof. FIG. 82a illustrates, for example, that when the device 100 is attached to the connector 262, the tube 258 and the camera 114 can be separated by the gap G3, the tube 258 and the deflectable section 108ds can be separated by the gap G3, and the tube 258 and the body 106 can be separated by the gap G3. The gap G3 can be, for example, 0.0 mm to 10.0 mm, or more narrowly, 0.0 mm to 5.0 mm, including every 0.1 mm increment within these ranges (e.g., 0.0 mm, 1.0 mm, 2.0 mm, 5.0 mm, 10.0 mm) For example, FIG. 82a illustrates that the gap G1 can be 1.5 mm. When the gap G3 is 0.0 mm, the tube 258 can contact the device 100 when the device is connected to the connector 262.

The gap G1, the gap G2, and the gap G3 can also be referred to as the first gap G1, the second gap G2, and the third gap G3, respectively.

FIG. 82b illustrates a front perspective view of FIG. 82a. FIG. 82b illustrates that the tube 260 can have an opening 260o. The opening 260o can be the distal opening to the lumen 261 of the tube 260. The lumen 261 can terminate at the opening 260o. FIG. 82b illustrates that the mount 262a can be removably attached to the tube 254. The mount 262a can be, for example, a clip or a hook. For example, FIG. 82b illustrates that the mount 262a can be a clip that can be removably clipped to the tube 254. FIG. 82b illustrates that the mount 262b can be removably attached to the tube 254. The mount 262b can be, for example, a clip or a hook. For example, FIG. 82b illustrates that the mount 262b can be a clip that can be removably clipped to the tube 254. As another example, the mount 262a and the mount 262b can be fixedly attached to the tube 254 such that the mount 262a and the mount 262b cannot be removed from the tube 254.

FIGS. 82a and 82b illustrate that the camera 114 can operate (e.g., take pictures and//or video) when the device 100 and the device 250 are in the configurations shown.

FIGS. 82a and 82b illustrate that the body cavity 142 can be, for example, the small intestine, and that the target site 144 can be, for example, the ampulla of Vater.

FIG. 83a illustrates a closeup of section 80x in FIG. 80 with the device 100 and the device 250 in the configurations shown in FIG. 80 with the device 100 and the device 250 in a body cavity 142 at a target site 144. FIG. 83a illustrates, for example, the device 100 and the device 250 in FIGS. 82a and 82b with the stabilizer 148 in a deployed configuration and with the tube 260 in a deployed configuration.

FIG. 83a illustrates the stabilizer 148 in a deployed configuration. The stabilizer 148 can be partially deployed (e.g., partially advanced) or fully deployed (e.g., fully advanced). The deployed configuration can be a partially advanced position or a fully advanced position of the tube 260. For example, FIG. 83a illustrates the stabilizer 148 in a fully advanced position.

FIG. 83a illustrates that when the stabilizer 148 is in a deployed configuration (e.g., in a partially deployed configuration and in a fully deployed configuration), the stabilizer 148 can extend from the tube 254 and/or from the connector 262.

FIG. 83a illustrates the tube 260 in a deployed configuration. The tube 260 can be partially deployed (e.g., partially advanced) or fully deployed (e.g., fully advanced). The deployed configuration can be a partially advanced position or a fully advanced position of the tube 260. For example, FIG. 83a illustrates the tube 260 in a partially advanced position. As another example, FIG. 83a can illustrate the tube 260 in a fully advanced position.

FIG. 83a illustrates that when the tube 260 is in a deployed configuration (e.g., in a partially deployed configuration and in a fully deployed configuration), the tube 260 can be deflected (e.g., via the control 268). For example, FIG. 83a illustrates that the tube 260 can be moved back and forth between the non-deflected configuration 278 and the deflected configuration 280. FIG. 83a illustrates, for example, the tube 260 in a deployed, non-deflected configuration (e.g., the non-deflected configuration 278) and in a deployed, deflected configuration (e.g., the deflected configuration 280).

FIG. 83a illustrates that the tube 260 can be deflected to align the tip 260t (e.g., the opening 260o) with the target site 144, for example, so that when the object 288 is advanced (e.g., as shown in FIG. 83a), the object 288 can be aligned with the target site 144 (e.g., as shown in FIG. 83a). The deflectable section 108ds can be articulated to align the camera 114 (e.g., to align the field of view of the camera 114) with the target site 144. The deflectable section 108ds can be articulated to align the tip 260t (e.g., the opening 260o) with the target site 144. The tube 260 (e.g., the deflectable section 260ds) and/or the deflectable section 108ds can be deflected to align the tip 260t (e.g., the opening 260o) and/or the camera 114 with the target site 144.

FIG. 83a illustrates an object 288 that can be advanced and retracted from the tube 260. The object 288 can be advanced and retracted from the tube 260 when the tube 260 is in a non-deflected configuration, in a deflected configuration, in a retracted position, in an advanced position, or any combination thereof.

FIG. 83a illustrates an object 288 in an advanced position. FIG. 83a illustrates that when the object 288 is in an advanced position, the distal end of the object 288 can be outside of the tube 260 and inside the body cavity 142.

FIG. 83b illustrates a front perspective view of FIG. 83a. FIG. 83b illustrates the object 288 of FIG. 83a in a retracted position. FIG. 83b illustrates that when the object 288 is in a retracted position, the distal end of the object 288 can be inside the tube 260, for example, inside the tip 260t, the deflectable section 260ds, and/or inside the portion of the tube 260 proximal the deflectable section 260ds. As another example, FIG. 83b can illustrate the object 288 completely withdrawn from the tube 260.

FIGS. 83a and 83b illustrate that the camera 114 can operate (e.g., take pictures and//or video) when the device 100 and the device 250 are in the configurations shown.

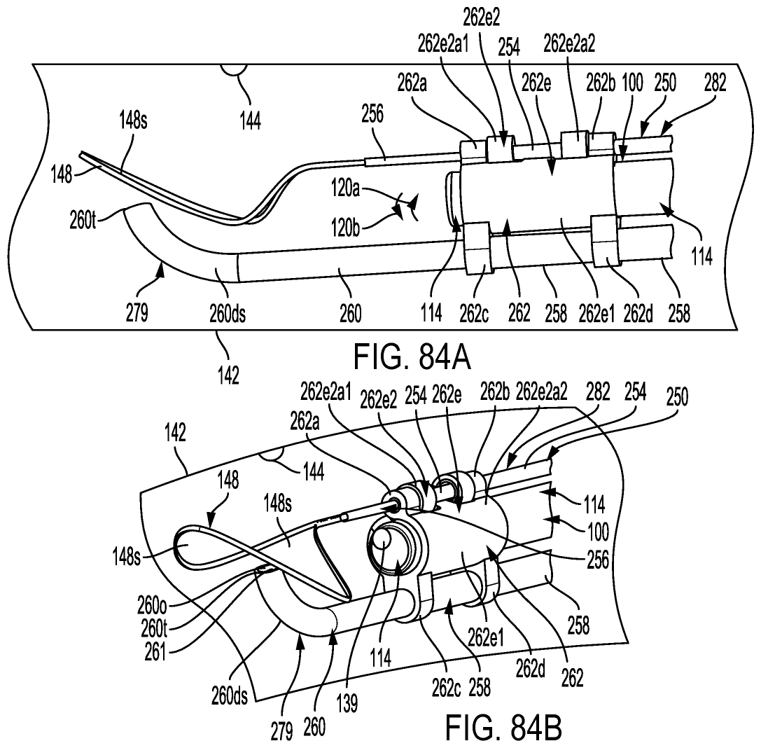

FIG. 84a illustrates a rear perspective view of section 80x in FIG. 80 with the device 100 and the device 250 in the configurations shown with the device 100 and the device 250 in a body cavity 142 at a target site 144. FIG. 84a illustrates, for example, the device 100 and the device 250 in FIGS. 83a and 83b with the stabilizer 148 in the deployed configuration (e.g., advanced position) shown in FIGS. 83a and 83b and with the tube 260 in the deployed configuration (e.g., advanced position) shown in FIGS. 83a and 83b. FIG. 84a illustrates that the deflectable section 260ds can have a deflected configuration 279 between the non-deflected configuration 278 and the deflected configuration. The tube 260 can have any configuration, for example, between the non-deflected configuration 278 and the deflected configuration 280. For example, FIG. 84a illustrates that the tube 260 can have the deflected configuration 279.

FIG. 84a illustrates that when the tube 260 is in the deflected configuration 279, the tube 260 (e.g., the opening 260o) may not be aligned with the target site 144, whereby the user can, for example, from the position shown in FIG. 84a, retract the system 282 in the body cavity 142 to align the tube 260 (e.g., the opening 260o) with the target site 144) and/or can articulate the deflectable section 260ds via the control 268 from the deflected configuration 279 to the deflected configuration 280. As another example, the tube 260 (e.g., the opening 260o) can be aligned with the target site 144 when the tube 260 is in the deflected configuration. The non-deflected configuration 278, the deflected configuration 279, and the deflected configuration 280 can also be referred to as the non-deflected configuration 278, the first deflected configuration 279, and the second deflected configuration 280, respectively. The non-deflected configuration 278, the deflected configuration 279, and the deflected configuration 280 can also be referred to as the first configuration 278, the second configuration 279, and the third configuration 280, respectively.

FIG. 84b illustrates a perspective view of FIG. 84a. FIG. 84b illustrates that the lens 139 can be offset from the longitudinal center of the camera 114.

FIGS. 84a and 84b illustrate that the connector 262e2 can have a first arm 262e2a1 and a second arm 262e2a2 that can be attached to (e.g., clipped to, hooked on, and/or wrapped around) the tube 254.

FIGS. 85a-85d illustrate the stabilizer 148 being progressively deployed from the tube 254 from FIG. 85a to FIG. 85d, and illustrate the stabilizer 148 being progressively retracted into the tube 254 from FIG. 85d to FIG. 85a. The stabilizer 148 can tension tissue. The stabilizer 148 can expand the body cavity 142. As the stabilizer 148 is deployed, the stabilizer 148 can automatically expand, for example, to the shape shown in FIG. 85d. The stabilizer 148 can be, for example, made from a shape memory metal or alloy (e.g., Nitinol) that can assume an expanded configuration when advanced from the tube 254. FIGS. 85a-85d illustrate the rest of the device 250 and the device 100 shown transparent so that the details of stabilizer 148 and the tube 254 can be more easily seen. As another example, FIGS. 85a-85d illustrate that the stabilizer 148 and the tube 254 can be deployed to the body cavity 142 and the target site 144 without the device 100 and/or without the rest of the device 250. As another example, FIGS. 85a-85d can illustrate the extension and retraction of the stabilizer from the tube 254 from the device 250, for example, as the connector 256 is advanced and retracted (e.g., via the control 264)

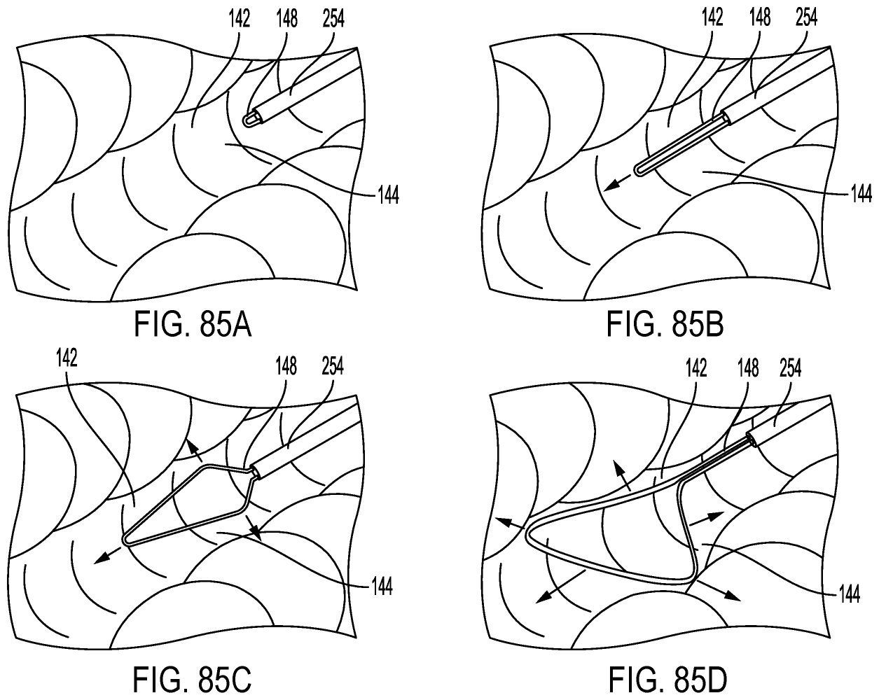

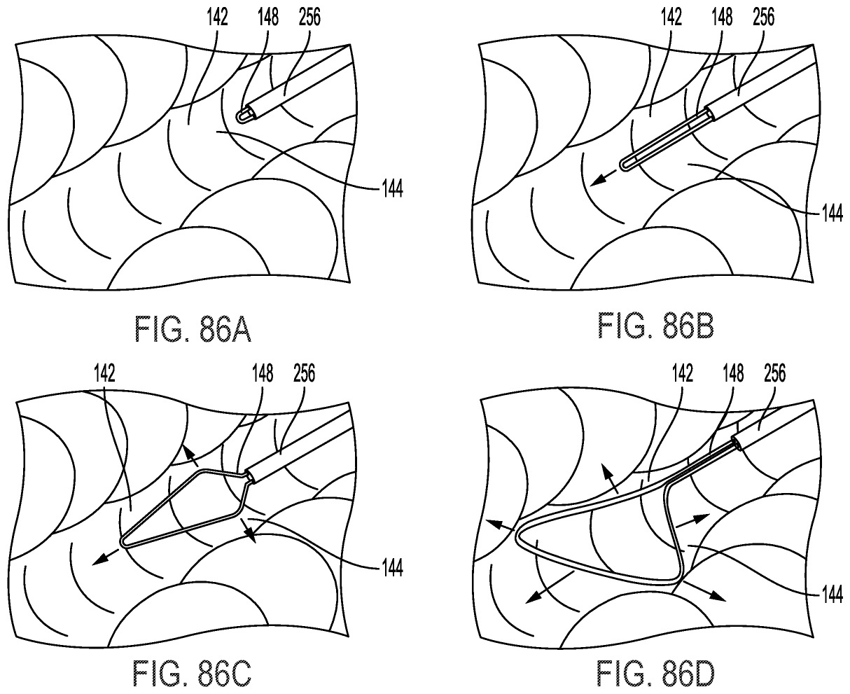

FIGS. 86a-86d illustrate the stabilizer 148 being progressively deployed from the connector 256 from FIG. 86a to FIG. 86d, and illustrate the stabilizer 148 being progressively retracted into the connector 256 from FIG. 86d to FIG. 86a. FIGS. 86a-86d illustrate the rest of the device 250 and the device 100 shown transparent so that the details of stabilizer 148 and the connector 256 can be more easily seen. As another example, FIGS. 86a-86d illustrate that the stabilizer 148 and the connector 256 can be deployed to the body cavity 142 and the target site 144 without the device 100 and/or without the rest of the device 250.

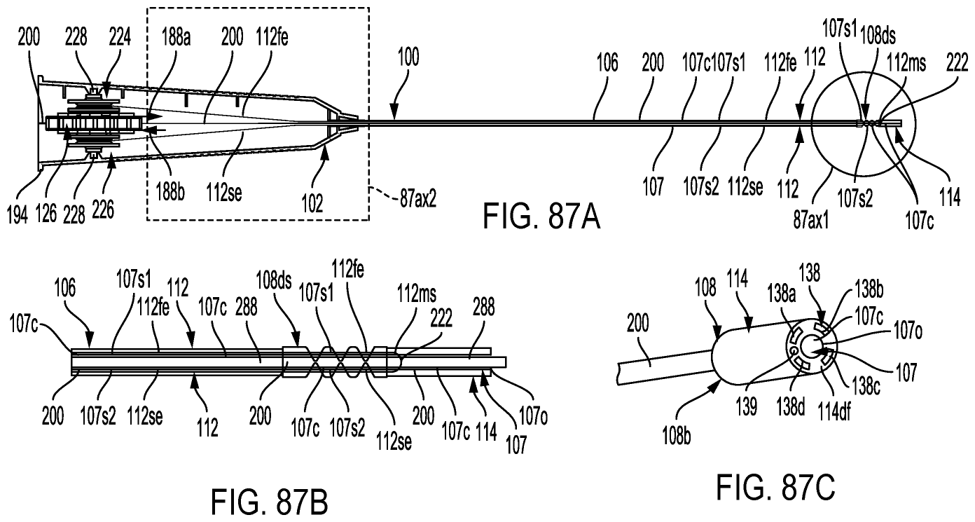

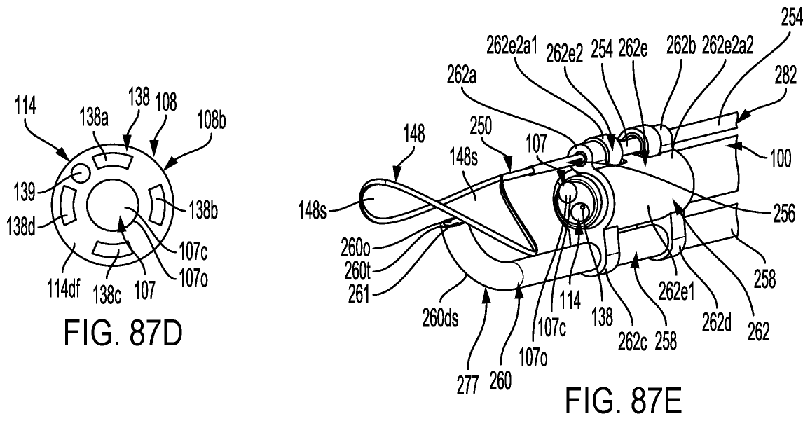

FIGS. 87a-87d illustrate that a lumen 107 (e.g., the central lumen 107c) can extend through the body 106, through the deflectable section 108ds, and through the camera 114 of the device 100. FIGS. 87a-87d illustrate that the lumen 107 (e.g., the central lumen 107c) can terminate at an opening 107o. The opening 107o can be the distal opening to the lumen 107 (e.g., the central lumen 107c) of the body 106, the deflectable section 108ds, and the camera 114. FIGS. 87a and 87b illustrate, for example, the device 100 in FIGS. 60 and 61 with a camera 114 in which the lumen (e.g., the central lumen 107c) extends through the camera 114. FIGS. 87a and 87b illustrate that the lumen 107 (e.g., the central lumen 107c) can be a working channel that objects 288 can be advanced in and retracted from.

FIG. 87b illustrates a closeup of section 87ax1 in FIG. 87a. FIG. 87b illustrates that the connector 200 and an object 288 can extend through the lumen (e.g., the central lumen 107c). FIG. 87b illustrates that the object 288 can be advanced from and/or retracted into the opening 107o.

FIGS. 87c and 87d illustrate the camera 114 in FIGS. 87a and 87b.

FIGS. 87a-87e illustrate that the central lumen 107c and the camera 114 can be offset from the longitudinal axis of the camera 114. FIG. 87e illustrates that the opening 107o and the camera 114 can be offset from the longitudinal axis of the camera 114. The central lumen 107c of the device 100 may not be a central lumen. For example, the central lumen 107c can be a side lumen. As another example, all the lumens 107, including the central lumen 107c, can be side lumens, whereby the device 100 may not have a lumen that is central relative to the rest of the lumens 107.

FIG. 87e illustrates that when the device 100 is attached to the device 250, the system 282 can have two working channels, for example, one of the lumens 107 (e.g., the central lumen 107c) and the lumen 261. During operation of the system 282, an object 288 can be inserted into and withdrawn from the central lumen 107c and an object 288 can be inserted into and withdrawn the lumen 261. The objects 288 can be advanced and retracted in the central lumen 107c and the lumen 261 independently of each other. For example, an object 288 can be advanced from and retracted into the opening 107o, and an object 288 can be advanced from and retracted into the opening 260o. Two objects 288 (e.g., a first object 288 and a second object 288) can thereby be advanceable and retractable from the system 282 (e.g., from the opening 107o and/or from the opening 260o) at the same time or at different times.

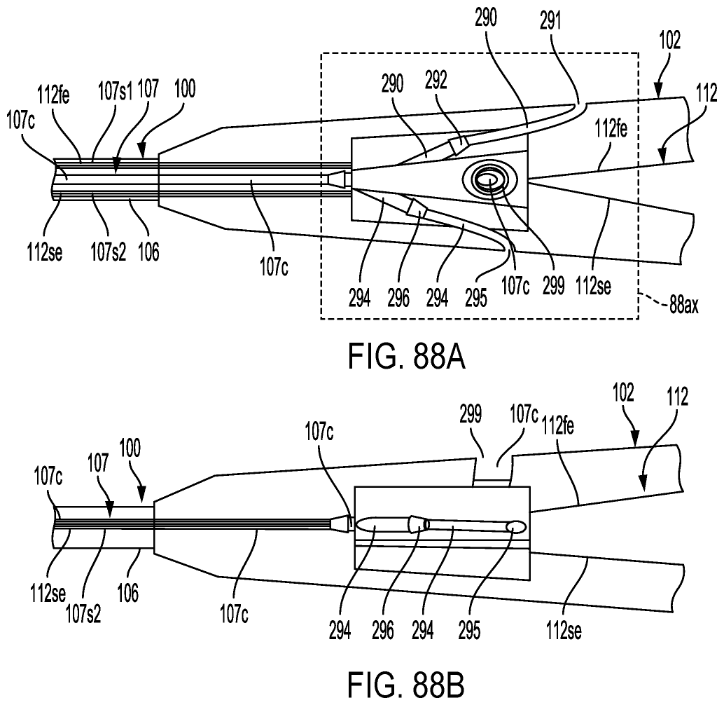

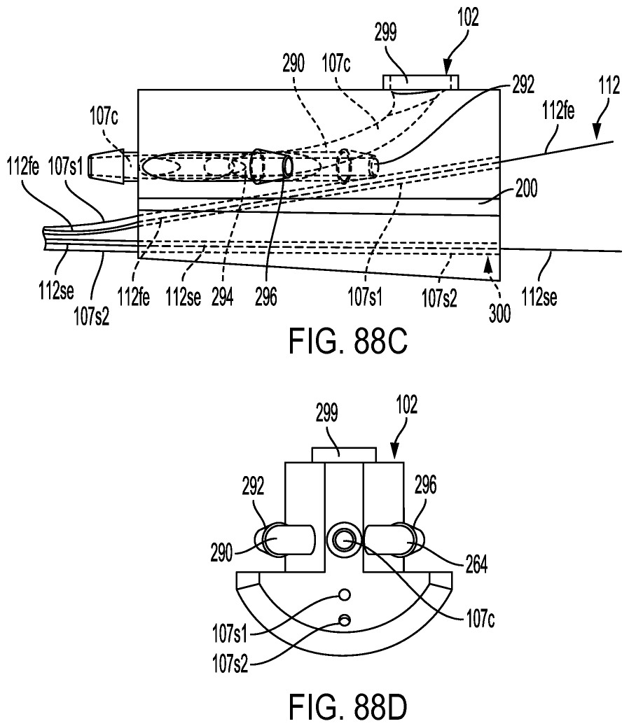

FIGS. 88a-88d illustrate a variation of a proximal end of the handle 102. For example, FIGS. 88a-88d illustrate a variation of the portion of the handle 102 that is proximal the control 126 in FIG. 87a. For example, FIGS. 88a and 88b illustrate a variation of features that the handle 102 can have in section 87ax2 in FIG. 87a.

FIG. 88a illustrates that the lumens 107 (e.g., the first side lumen 107s1, the second side lumen 107s2, and the central lumen 107c) of the body 106 can extend into the handle 102. FIG. 88a illustrates that the central lumen 107c can terminate at an opening 299. The opening 299 can be a port on the surface of the handle 102. For example, FIG. 88a illustrates that the opening 299 can be a port on the top surface of the handle 102. The opening 299 can be the proximal opening to the central lumen 107c. Objects 288 can be inserted into the central lumen 107c and withdrawn from the central lumen 107c via the opening 299. The opening 299 can be anywhere on the handle 102.

FIG. 88a illustrates that the handle 102 can have a fluid channel 290 connected to the central lumen 107c. The fluid channel 290 can have an exterior port 291. The exterior port 291 can be on a surface of the handle 102. The fluid channel 290 can be, for example, an irrigation channel. The fluid channel 290 can be, for example, a water channel. A valve 292 (e.g., a one-way valve) can connect or be between the central lumen 107c and the fluid channel 290. FIG. 88a illustrates that the handle 102 can have a suction channel 294 connected to the central lumen 107c. The suction channel 294 can have an exterior port 295. The exterior port 295 can be on a surface of the handle 102. A valve 296 (e.g., a one-way valve) can connect or be between the central lumen 107c and the suction channel 294. FIG. 88a illustrates that the central lumen 107c can extend through the body 106, the deflectable section 108ds, and/or the camera 114. FIG. 88a illustrates, for example, a top view of the handle 102. As another example, the handle 102 may not have the valve 292 and/or may not have the valve 294. In such cases, the valve 292 can instead be a connector (e.g., a barb or luer) between two different sized lumens to connect the exterior port 291 to the central lumen 107c, and the valve 294 can instead be a connector (e.g., a barb or luer) between two different sized lumens to connect the exterior port 295 to the central lumen 107c.

FIG. 88b illustrates that the opening 299 can be an opening in the wall of the handle 102. FIG. 88b illustrates, for example, a side view of the handle 102 of FIG. 88a.

FIG. 88c illustrates that the handle 102 can have the first side lumen 107s1 and the second side lumen 107s2. FIG. 88c illustrates, for example, that the first side lumen 107s1 and the second side lumen 107s2 can extend into the handle 102. FIG. 88c illustrates, for example, a side view of section 88ax in FIG. 88a.

FIG. 88d illustrates, for example, a front view of the features in section 88ax in FIG. 88a.

The first size 244a of the device 100 can have any combination of features disclosed herein. The first size 244a of the device 100 can be any of the devices 100 disclosed and/or illustrated herein and can have any of the features disclosed and/or illustrated herein. For example, the first size 244a of the device 100 can be the device 100 shown in FIG. 60. The first size 244a of the device 100 can be removably attached to the device 250, for example, via the connector 262.

The second size 244b of the device 100 can have any combination of features disclosed herein. The second size 244b of the device 100 can be any of the devices 100 disclosed and/or illustrated herein and/or can have any of the features disclosed and/or illustrated herein. For example, the second size 244b of the device 100 can be the device 100 shown in FIGS. 87a-88d. The second size 244b of the device 100 can be removably attached to the device 250, for example, via the connector 262.

The third size 244c of the device 100 can have any combination of features disclosed herein. The third size 244c of the device 100 can be any of the devices 100 disclosed and/or illustrated herein and/or can have any of the features disclosed and/or illustrated herein. For example, the third size 244c of the device 100 can be the device 100 shown in FIGS. 87a-88d. The third size 244c of the device 100 can be removably attached to the device 250, for example, via the connector 262.

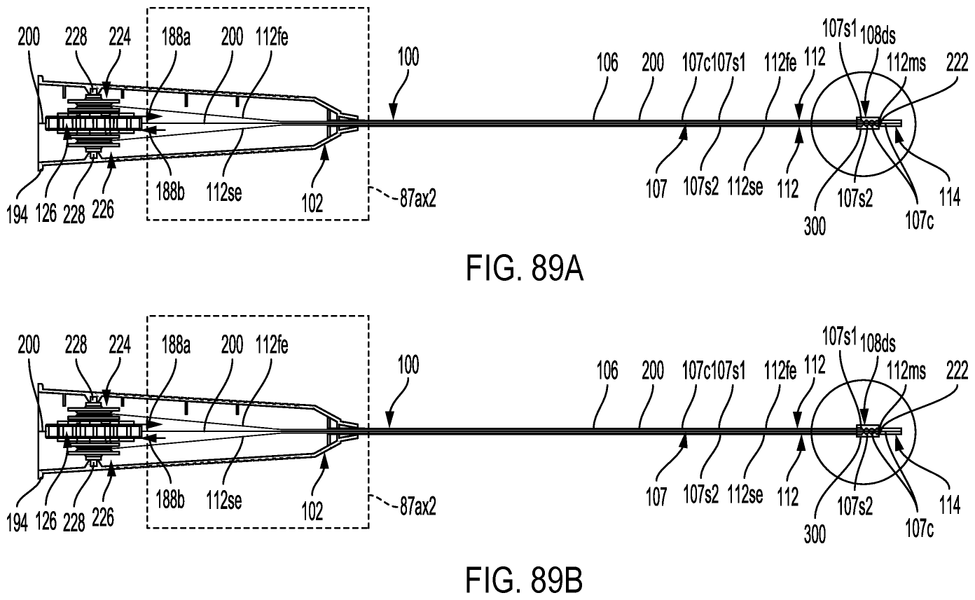

FIG. 89a illustrates that the tip 108 can have a cover 300. The cover 300 can be fixedly attached to the tip 108. The cover 300 can be removably attached to the tip 108. The cover 300 can be flexible, for example, so that the deflectable section 108ds can be articulated in the first and second directions 120a, 120b (e.g., via the control 126) while the cover 300 is on the device 100 (e.g., on the tip 100). The cover 300 can be, for example, heat shrink. For example, FIG. 89a illustrates that the deflectable section 108ds can have the cover 300. The cover 300 can surround the outside of the deflectable section 108ds, for example, to prevent anything from entering the lumens 107 (e.g., the first side lumen 107s1, the second side lumen 107s2, and the central lumen 107c) that are exposed on the faces 218 of the deflectable section 108ds. For example, the cover 300 can be a sleeve or a tube of heat shrink. The cover 300 can cover the exposed ports of the lumens 107 in the deflectable section 108. The cover 300 can prevent fluid and/or debris from entering the device 100 through the tip 108. The cover 300 can thereby be a barrier between the environment (e.g., the body cavity 142) and the tip 108 (e.g., the deflectable section 108ds). The cover 300 can thereby be a barrier between the environment (e.g., the body cavity 142) and the lumens 107 in the tip 108. The cover can be transparent or opaque. For example, FIG. 89a illustrates that the cover 300 can be transparent.

FIG. 89b illustrates that the cover 300 can cover the deflectable section 108ds and the camera 114. FIG. 89b illustrates that when the cover 300 covers the camera 114, the cover 300 may not cover the camera lens 139. As another example, the cover 300 can cover the camera lens 139.

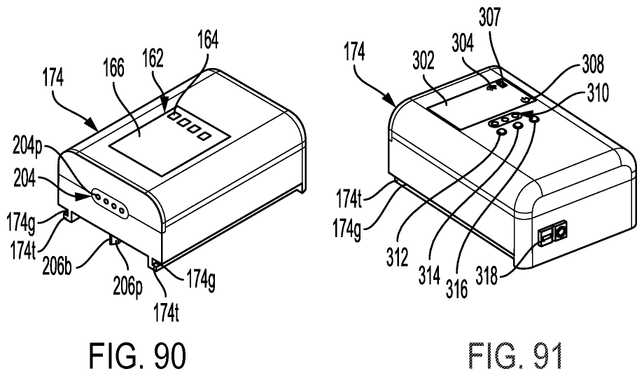

FIG. 90 illustrates that the module 174 can have a user interface 162. The user interface 162 can have controls 164 and an electronic display 166. FIG. 90 illustrates that that the electronic display 166 can be a touchscreen and that the controls 164 can be electronic controls on the touchscreen. As another example, the controls 164 can be physical controls (e.g., buttons, switches, knobs). The controls 164 can control the camera 114. The controls 164 can turn the camera 114 on, turn the camera 114 off, can control the illuminators (e.g., on, off, white balance, color, brightness). The display 166 can be, for example, a light-emitting diode (LED) screen or an organic light-emitting diode (OLED) screen. The display 166 can, for example, provide status feedback. The handle 102 can have connections for water instillation. The handle 102 can have controls for the stabilizer 148 (e.g., to advance and retract the stabilizer 148). The display 166 can show the user images of the camera 114 and/or other visualizations of the body cavity that the device is in (e.g., see FIGS. 82a-84b) in real time, for example, images of captured by the camera 114, images captured by a fluoroscopic imaging technique, or both, that can allow the user to view the body cavity and location of the device 100.

FIG. 91 illustrates that the module 174 can have a display 302, an indicator 304, an indicator 307, an indicator 308, status indicators 310, a control 312, a control 314, a control 316, a control 318, or any combination thereof.

The display 302 can be an electronic display. For example, the display 302 can be a light-emitting diode (LED) screen or an organic light-emitting diode (OLED) screen. For example, FIG. 91 illustrates that the display 302 can be an LED screen. The display 302 can be a touchscreen. As another example, the display 302 may not be a touchscreen.

The indicators 304, 307, and/or 308 can be indicators on the display 302. The indicators 304, 307, and/or 308 can be displayable on the display 302. The indicators 304, 307, and/or 308 can be indicators that are displayable on the display 302.

The indicator 304 can be, for example, a WiFi indicator. The indicator 304 can, for example, indicate that the device 100 (e.g., the module 174 and/or the camera 114) is connected to WiFi, can indicate that the device 100 (e.g., the module 174 and/or the camera 114) is not connected to WiFi, can indicate the strength of a WiFi signal, or any combination thereof.

The indicator 307 can be, for example, a battery indicator. The indicator 307 can, for example, indicate the battery level remaining. The indicator 307 can be, for example, a battery icon that indicates the batter level remaining.

The indicator 308 can indicate that the device 100 (e.g., the module 174 and/or the camera 114) is powered on, and can indicate that the device 100 (e.g., the module 174 and/or the camera 114) is powered off.

The status indicators 310 can be status light-emitting diodes (LEDs). The status indicators 310 can indicate, for example, the brightness level of the illuminators 138, the strobe frequency of the illuminators 138, and/or the color of the illuminators 138 (e.g., white, green, red, and/or blue). The module 174 can have, for example, 1-10 status indicators 310, including every 1 increment within this range (e.g., 1 status indicator, 5 status indicators, 10 status indicators). For example, FIG. 91 illustrates that the module 174 can have 3 status indicators 310.

The controls 312, 314, 316, and 318 can control any feature of the device 100, of the device 250, and/or of the system 282. The controls 312, 314, 316, and 318 can be, for example, buttons, knobs, switches, wheels, or any combination thereof. For example, FIG. 91 illustrates that the controls the controls 312, 314, and 316 can be buttons, and that the control 318 can be a switch.

For example, the control 312 can turn the illuminators 138 on and off, can selectively turn on and/or illuminators 138 (e.g., white, red, blue, green illuminators) by pressing the control 312 multiple times, can turn on and off a strobe frequency of the illuminators 138, or any combination thereof.

The control 314 can be, for example, a menu button for the display 302, for example, that can be used to cycle through different options displayed on the display 302.

The control 316 can be, for example, a selection button for the display 302, for example, to select an option on the display (e.g., white balance, color, brightness of the illuminators 138).

The control 318 can be, for example, an on/off switch for the device 100 (e.g., for the module 174 and/or for the camera 114).

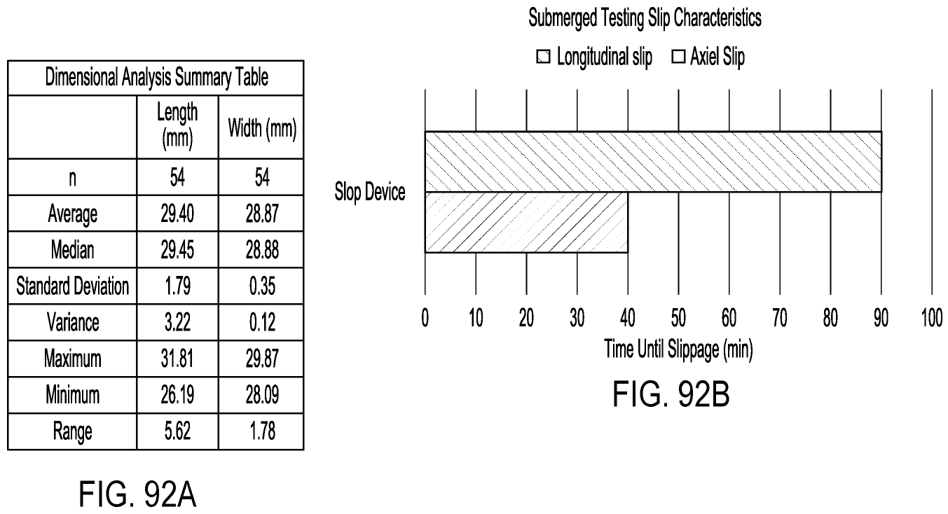

FIGS. 92a and 92b illustrate testing data of the device 250 and stability (non-slip) testing results. FIG. 92a illustrates a dimensional analysis summary table and FIG. 92b illustrates the stability (non-slip) testing results. Dimensional measurements were recorded for length and width. Submerged testing was performed to determine slip time between the device 250 and the device 100 (e.g., the slip time between the connector 262 and the device 100). The testing was stopped at 90 minutes. No longitudinal slippage occurred between the device 250 and the device 100. No longitudinal slippage occurred between the connector 262 and the device 100. The testing was performed on GMP and GMP-like sterile devices.

Any of the components of the device 100 and/or any of the components of the device 250 can be 3D printed parts and/or molded parts.

The device 100 can be disposable. The device 100 can be disposable and reusable. The device 100 can have, for example, reusable components and/or disposable components. For example, the handle 102 can be disposable, the body 106 can be disposable, the tip 108 can be disposable, the camera 114 can be disposable, the module 174 can be reusable, or any combination thereof. As another example, the entire device 100 can be disposable. For example, the handle 102 can be disposable, the body 106 can be disposable, the tip 108 can be disposable, the camera 114 can be disposable, and the module 174 can be disposable. As yet another example, the entire device 100 can be reusable.

The device 250 can be disposable. The device 250 can be disposable and reusable. The device 250 can have, for example, reusable components and/or disposable components. For example, the handle 252 can be reusable (e.g., the tube 254 and the tube 258 can be removably attachable to the handle 252 so that the handle 252 can be reusable), the tube 254 can be disposable, the connector 256 can be disposable, the stabilizer 148 can be disposable, the tube 258 can be disposable, the tube 260 can be disposable, the connector 262 can be disposable, or any combination thereof. As another example, the entire device 250 can be disposable. For example, the handle 252 can be disposable, the tube 254 can be disposable, the connector 256 can be disposable, the stabilizer 148 can be disposable, the tube 258 can be disposable, the tube 260 can be disposable, and the connector 262 can be disposable.

As yet another example, the entire device 250 can be reusable.

The system 282 can be disposable. For example, the device 100 and the device 250 of the system 282 can be disposable. As another example, the system 282 can be reusable. For example, the module 174, the handle 252, the computer 284, or any combination thereof of the system 282 can be reusable. For example, the module 174 and the computer 284 can be reusable.

Any of the features disclosed, contemplated, and/or illustrated herein can be combined in any combination with each other. For example, the device 100 can have any of the cameras 114 or any combination of the cameras 114 disclosed herein. As another example, the device 250 can have any of the cameras 114 or any combination of the cameras 114 disclosed herein.

FIGS. 1-91 illustrate, for example, a guidewire (e.g., the guidewire 178) can be advanced to any location in the body (e.g., to the target site 144 in the body cavity 142 shown in FIG. 39). The body cavity 144 can be, for example, the intestine. For example, the guidewire (e.g., the guidewire 178) can be passed through an obstruction (e.g., a small bowel obstruction). The device 100 can then be slid over the guidewire (e.g., as shown in FIG. 40) and once the device 100 is in the correct position, the guidewire can be removed and a torque member (e.g., the torque transmitter 110) can be inserted into the device 100 (e.g., as shown in FIG. 41). The torque member can be inserted through a channel (e.g., a central channel such as a lumen 107) and can engage with the distal end of the device 100 (e.g., with the distal end of the body 106 and/or with the tip 108). The torque member (e.g., the torque transmitter 110) can allow the device 100 (e.g., endoscope) to be twisted and rotated as desired while the device is in the body (e.g., at the target site 144 in the body cavity 142 as shown in FIG. 41) to examine the body cavity 142. The torque transmitter 110 can allow the user to control the body 106 and the tip 108, for example, by providing a 1:1 torque ratio when the torque transmitter 110 is inserted in the device 100. The device 100 can be advanced over the guidewire (e.g., the guidewire 178) to the obstruction (e.g., a small bowel obstruction). The device 100 can be used to clear an obstruction (e.g., a small bowel obstruction) in the body cavity 142 with or without balloon dilation. The colon can be shortened and stabilized, for example, using the device 100.

The device 100 can allow a user to use various tools in the body cavity, can allow the user to stabilize the device 100 in the body cavity, and can allow the user to perform colon shortening without losing their place in the bowel. The device 100 can be used as a colonoscope, a gastroscope, or both. As another example, the device 100 can be a colonscope and a gastroscope can be passed through a lumen (e.g., one of the lumens 107) of the device. The handle 102 can allow the user to place tools and can have a steering component (e.g., a control or controls) for the actuators 112. The handle 102 can have an electronics module (e.g., the module 174, the module 180) that can be removably connectable to the end of the handle 102.

FIGS. 1-91 illustrate that the handle 102 can removably engage with the body 106. The body 106 can be, for example, a disposable catheter. The handle 102 and the body 106 can be separately disposable. The electronics module (e.g., the module 174) can be reusable and can be removed from the handle 102 before disposing of the handle 102. As another example, the handle 102 and the electronics module (e.g., the module 174) can be disposable (e.g., separately disposable). The user can disconnect the electronics module (e.g., the module 174), can disconnect the handle 102, can put fluid, air, or contrast in through a lumen (e.g., one or multiple lumens 107), can take out the torque member (e.g., the torque transmitter 110) if the user does not need the device 100 to be so stiff, can put the guidewire (e.g., the guidewire 178) in the device 100 (e.g., pass the guidewire 178 through the body 106) all the way until the guidewire comes out the distal end of the tip 108, for example, to keep their place inside the body cavity 142 (e.g., in the intestine) when the user removes the body 106 (e.g., the catheter), and then can but put in other tools or devices as desired (e.g., a short unit as needed). The modular system shown in FIGS. 1-91 can, for example, advantageously give the user flexibility during procedures, allow the user to plan more efficient procedures, all the user to more quickly respond to the anatomical conditions of the patient (e.g., of the obstruction or blockage) during use, or any combination thereof, in addition to or in any combination with any of the other benefits detailed herein (e.g., stabilization).

The features of the device 100 can include, for example, any combination of features described herein and/or shown in FIGS. 1-91. For example, the device 100 can have any combination of features in FIGS. 44-91 and/or described in relation thereto. FIGS. 44-91 illustrate, for example, an access device (e.g., the device 100) can have a tube (e.g., the body 106), a deflectable section (e.g., the deflectable section 108ds), and a camera (e.g., the camera 114). The deflectable section can be movable from a first configuration to a second configuration. When the deflectable section is in the first configuration, the deflectable section can be straight, and when the deflectable section is in the second configuration, the deflectable section can have a curve. When the deflectable section is in the first configuration, the deflectable section can be less curved than when the deflectable section is in the second configuration. The deflectable section can be movable from the second configuration to the first configuration. The deflectable section can be movable from the first configuration or the second configuration to a third configuration. The deflectable section can be movable from the third configuration to the first configuration or the second configuration. When the deflectable section is in the first configuration, the deflectable section can be straight, when the deflectable section is in the second configuration, the deflectable section can have a first curve, and when the deflectable section is in the third configuration, the deflectable section can have a second curve. When the deflectable section has the first curve, the deflectable section can have a partially deflected configuration. When the deflectable section has the first curve, the deflectable section can have a fully deflected configuration. When the deflectable section has the second curve, the deflectable section can have a partially deflected configuration. When the deflectable section has the second curve, the deflectable section can have a fully deflected configuration. The second configuration of the deflectable section can be a partially deflected configuration of the deflectable section. The second configuration of the deflectable section can be a fully deflected configuration of the deflectable section. The third configuration of the deflectable section can be a partially deflected configuration of the deflectable section. The third configuration of the deflectable section can be a fully deflected configuration of the deflectable section. The radius of curvature of the first curve can be the same as the radius of curvature of the second curve. The radius of curvature of the first curve can be different than the radius of curvature of the second curve. The radius of curvature of the first curve can be greater than the radius of curvature of the second curve. The radius of curvature of the first curve can be less than the radius of curvature of the second curve. The device can have a handle (e.g., the handle 102). The handle can have a control (e.g., the control 126), and the deflectable section can be movable from the first configuration to the second configuration via the control. The handle can have a control (e.g., the control 126), and the deflectable section can be movable from the second configuration to the first configuration via the control. The handle can have a control (e.g., the control 126), and the deflectable section can be movable from the first configuration or the second configuration to the third configuration via the control. The handle can have a control (e.g., the control 126), and the deflectable section can be movable from the third configuration to the first configuration or the second configuration via the control. The device can have a handle (e.g., the handle 102) and a module (e.g., the module 174), and the module can be removably connectable to the handle. The module can be an electronics module. When the module is removably connected to the handle, the camera is powerable by a battery in the electronics module. The module (e.g., the module 174) can be reusable, and the tube, the deflectable section, and/or the camera can be disposable. The camera can be coverable with a cover (e.g., the cover 300). The cover can be a heat shrink. The deflectable section can have a hinge (e.g., a hinge 192), a first segment (e.g., the first segment 214a), and a second segment (e.g., the second segment 214b). The first segment can be rotatable relative to the second segment about the hinge, and the second segment can be rotatable relative to the first segment about the hinge. The hinge can be a living hinge. The first segment can be connected to the second segment via the hinge. The tube (e.g., the body 106 and/or the tip 108) comprises a wall, and the wall of the tube can comprise the hinge. A lumen can extend through the hinge. The hinge can be on a first side of the tube and on a second side of the tube. When the deflectable section is in the second configuration, the first segment and the second segment can be closer together than when the deflectable section is in the first configuration. When the deflectable section is in the third configuration, the first segment and the second segment can be closer together than when the deflectable section is in the first configuration. When the deflectable section is in the first configuration, the first segment and the second segment can be separated by a first gap, when the deflectable section is in the second configuration, the first segment and the second segment can be separated by a second gap, and the second gap can be smaller than the first gap. When the deflectable section is in the second configuration, the first segment and the second segment can be separated by the second gap, when the deflectable section is in the third configuration, the first segment and the second segment can be separated by a third gap, and the third gap can be smaller than the first gap. When the deflectable section is in the first configuration, the first segment and the second segment can be separated by a first gap, when the deflectable section is in the second configuration, the first segment and the second segment can be separated by a second gap, when the deflectable section is in the third configuration, the first segment and the second segment can be separated by the second gap, and the second gap can be smaller than the first gap. When the deflectable section is in the first configuration, the first segment and the second segment can be separated by a first space, when the deflectable section is in the second configuration, the first segment and the second segment can be separated by a second space, and wherein the second space can be smaller than the first space. When the deflectable section is in the third configuration, the first segment and the second segment can be separated by a third space, and the third space can be smaller than the first space. When the deflectable section is in the first configuration, the first segment and the second segment can be separated by a first space, when the deflectable section is in the second configuration, the first segment and the second segment can be separated by a second space, when the deflectable section is in the third configuration, the first segment and the second segment can be separated by the second space, and the second space can be smaller than the first space. The first segment and the second segment can be separated by a first space and a second space, the first space can be smaller when the deflectable section is in the second configuration than when the deflectable section is in the first configuration, and the second space can be larger when the deflectable section is in the second configuration than when the deflectable section is in the first configuration. The first space can be smaller when the deflectable section is in the third configuration than when the deflectable section is in the second configuration, and the second space can be larger when the deflectable section is in the third configuration than when the deflectable section is in the second configuration. The first segment and the second segment can be separated by a first space (e.g., space 216a) and a second space (e.g., space 216d), and when the deflectable section is in the second configuration, the first space can be smaller than the second space. When the deflectable section is in the first configuration, the first space can be the same size as the second space. When the deflectable section is in the third configuration, the first segment and the second segment can contact each other. The first segment can have a first segment first surface (e.g., surface 218a) and a first segment second surface (e.g., surface 218g), the second segment can have a second segment first surface (e.g., surface 218b) and a second segment second surface (e.g., surface 218h), an angle (e.g., angle 220) between the first segment first surface and the second segment first surface can be less when the deflectable section is in the second configuration than when the deflectable section is in the first configuration. An angle (e.g., angle 220) between the first segment second surface and the second segment second surface can be greater when the deflectable section is in the second configuration than when the deflectable section is in the first configuration. The angle between the first segment first surface and the second segment first surface can be less when the deflectable section is in the third configuration than when the deflectable section is in the second configuration. The angle between the first segment first surface and the second segment first surface can be greater when the deflectable section is in the third configuration than when the deflectable section is in the second configuration. The angle between the first segment second surface and the second segment second surface can be greater when the deflectable section is in the third configuration than when the deflectable section is in the second configuration. The angle between the first segment second surface and the second segment second surface can be less when the deflectable section is in the third configuration than when the deflectable section is in the second configuration. The first segment first surface and the second segment first surface can be adjacent to each other. The first segment second surface and the second segment second surface can be adjacent to each other. The first segment first surface and the second segment first surface can be on a first side of the deflectable section, and the first segment second surface and the second segment second surface can be on a second side of the deflectable section. The first side of the deflectable section can be opposite the second side of the deflectable section. When the deflectable section is in the first configuration, the first segment first surface can face the second segment first surface, and when the deflectable section is in the first configuration, the first segment second surface can face the second segment second surface. The deflectable section and/or the camera can be movable in a first direction (e.g., the first direction 120a) and in a second direction (e.g., the second direction 120b) via the control, and the first direction can be opposite the second direction. The deflectable section and/or the camera be rotatable in a first direction (e.g., the first direction 120a) and in a second direction (e.g., the second direction 120b) via the control, and the first direction is opposite the second direction. The tube can have a lumen (e.g., a lumen 107), and an object can be advanceable and retractable in the lumen. The lumen can extend through the deflectable section and the camera. The lumen can be a working channel. The access device can be an endoscope. The tube, the deflectable section, and the camera can comprise an endoscope.

The features of the device 250 can include, for example, any combination of features described herein and/or shown in FIGS. 1-91. For example, the device 250 can have any combination of features in FIGS. 79-91 and/or described in relation thereto. FIGS. 79-91 illustrate, for example, an access device (e.g., the device 250) can have a first tube (e.g., the tube 254) and a second tube (e.g., the tube 258). A stabilizer (e.g., the stabilizer 148) can be advanceable from the first tube. A third tube (e.g., the tube 260) can be advanceable from the second tube. The stabilizer can be retractable into the first tube. The third tube can be retractable into the second tube. The stabilizer can be selectively advanceable and retractable independently of the third tube. The third tube can be selectively advanceable and retractable independently of the stabilizer. When the stabilizer is inside the first tube, the stabilizer can have a contracted configuration. When the stabilizer is outside the first tube, the stabilizer can have an expanded configuration. When the stabilizer has the expanded configuration, the stabilizer can have an opening (e.g., the space 148s) that the third tube and/or an object can be movable through. When the stabilizer has the expanded configuration, the stabilizer can have an opening (e.g., the space 148s) that the third tube and/or an object can be advanceable and/or retractable through. The device 250 can have a connector (e.g., the connector 262). The first tube can be connected to the second tube via the connector. A connector (e.g., the connector 262) can be connected to the first tube and the second tube. The device 250 can have a connector (e.g., the connector 262). The first tube and the second tube can be removably connected to the connector. The device 250 can have a connector. The first tube and the second tube can be removably connected to the connector. The first tube can be connected to a first mount (e.g., mount 262a) and/or a second mount (e.g., mount 262b) of the connector, and the second tube can be connected to a third mount (e.g., mount 262c) and/or a fourth mount (e.g., mount 262d) of the connector. The second tube can be compressible. The second tube can be collapsible. The second tube can be collapsible when the second is pressed against a surface. The second tube can be collapsible when the second is in contact with a surface. The second tube can have an open configuration and a collapsed configuration. When the second tube is in the open configuration, a lumen in the second tube can be larger than when the second tube is in the collapsed configuration. When the second tube is in the collapsed configuration, the lumen can be partially collapsed or fully collapsed. When the second tube is fully collapsed, opposite sides of an inner surface of the second tube can contact each other. When the second tube has the open configuration, the third tube can be advanceable and retractable in the second tube. When the second tube has the collapsed configuration, the third tube can be advanceable in the lumen to open the second lumen. The third tube can have a third tube deflectable section (e.g., the deflectable section 260ds). The third tube deflectable section can be deflectable from a first configuration to a second configuration. The device 250 can have a handle (e.g., the handle 252) having a first control (e.g., the control 264) and a second control (e.g., the control 266). The stabilizer can be advanceable and retractable via the first control (e.g., the control 264). The third tube can be advanceable and retractable via the second control (e.g., the control 266). The handle can have a third control (e.g., the control 268). The third tube deflectable section can be deflectable via the third control. The third tube deflectable section can be deflectable from the first configuration to the second configuration via the third control.

The features of the system 282 can include, for example, any combination of features described herein and/or shown in FIGS. 1-91. For example, the device 250 can have any combination of features disclosed in FIGS. 79-91 and/or described in relation thereto. The A system (e.g., the system 282) can have a first device (e.g., the device 100) and a second device (e.g., the device 250). The first device can have any of the features disclosed herein (e.g., any of the features of FIGS. 1-91, for example, any of the features in paragraph [0415]. The second device can have any of the features disclosed herein (e.g., any of the features of FIGS. 1-91, for example, any of the features in paragraph [0416]). The first device can be removably attachable to the connector. The first device can be removably attachable to the second device via the connector. The first device can be removably attached to the connector, and wherein the second device is attached to the connector. The tube, the deflectable section, and/or the camera of the first device can be removably attachable to the connector. When the first device and the second device are attached to the connector, the stabilizer can be selectively advanceable and retractable independently of the advancement and retraction of the third tube. When the first device and the second device are attached to the connector, the stabilizer can be selectively advanceable and retractable independently of the deflection of the third tube deflectable section. When the first device and the second device are attached to the connector, the stabilizer can be selectively advanceable and retractable independently of the deflection of the deflectable section. When the first device and the second device are attached to the connector, the third tube can be selectively advanceable and retractable independently of the advancement and retraction of the stabilizer. When the first device and the second device are attached to the connector, the third tube can be selectively advanceable and retractable independently of the deflection of the third tube deflectable section. When the first device and the second device are attached to the connector, the third tube can be selectively advanceable and retractable independently of the deflection of the deflectable section. When the first device and the second device are attached to the connector, the third tube deflectable section can be selectively deflectable independently of the advancement and retraction of the stabilizer. When the first device and the second device are attached to the connector, the third tube deflectable section can be selectively deflectable independently of the advancement and retraction of the third tube. When the first device and the second device are attached to the connector, the third tube deflectable section can be selectively deflectable independently of the deflection of the deflectable section. When the first device and the second device are attached to the connector, the deflectable section can be selectively deflectable independently of the advancement and retraction of the stabilizer. When the first device and the second device are attached to the connector, the deflectable section can be selectively deflectable independently of the advancement and retraction of the third tube. When the first device and the second device are attached to the connector, the deflectable section can be selectively deflectable independently of the deflection the third tube deflectable section. The system can be an access system.

FIGS. 1-91 illustrate, for example, a method of articulating a tip (e.g., the tip 108) of an endoscope (e.g., the device 100). The method can include articulating a deflectable section (e.g., the deflectable section 108ds0 having a first segment (e.g., the first segment 214a), a second segment (e.g., the second segment 214b), and a hinge (e.g., the hinge 192).

FIGS. 1-91 illustrate, for example, a method of assembling and/or disassembling a system (e.g., 282) comprising attaching an endoscope (e.g., the device 100) to a connector (e.g., the connector 262) attached to a first tube (e.g., the tube 254 or the tube 258) and/or a second tube (e.g., the tube 258 or the tube 254) and/or detaching the endoscope from the connector attached to the first tube and/or the second tube.

FIGS. 1-91 illustrate, for example, a method of advancing and/or retracting a stabilizer (e.g., the stabilizer 148). The method can include advancing the stabilizer from a first tube (e.g., the tube 254). The first tube can be removably attached to the outside of a second tube (e.g., the body 106, the tip 108, the camera 114, and/or the tube 258). The method can include retracting the stabilizer into the first tube.

The claims are not limited to the exemplary variations shown in the drawings, but instead may claim any feature disclosed or contemplated in the disclosure as a whole. Any elements described herein as singular can be pluralized (i.e., anything described as “one” can be more than one). Any species element of a genus element can have the characteristics or elements of any other species element of that genus. Some elements may be absent from individual figures for reasons of illustrative clarity. The above-described configurations, elements or complete assemblies and methods and their elements for carrying out the disclosure, and variations of aspects of the disclosure can be combined and modified with each other in any combination, and each combination is hereby explicitly disclosed. All devices, apparatuses, systems, and methods described herein can be used for medical (e.g., diagnostic, therapeutic or rehabilitative) or non-medical purposes. The words “may” and “can” are interchangeable (e.g., “may” can be replaced with “can” and “can” can be replaced with “may”). Any range disclosed can include any subrange of the range disclosed, for example, a range of 1-10 units can include 2-10 units, 8-10 units, or any other subrange. Any phrase involving an “A and/or B” construction can mean (1) A alone, (2) B alone, (3) A and B together, or any combination of (1), (2), and/or (3), for example, (1) and (2), (1) and (3), (2) and (3), and (1), (2), and (3). The term about can include any tolerance that would be understood by one or ordinary skill in the art, for example, plus or minus 5% of the stated value.

We claim:

1. An access device comprising:
a tube;
a deflectable section; and
a camera,
wherein the deflectable section is movable from a first configuration to a second configuration, wherein when the deflectable section is in the first configuration, the deflectable section is straight, and wherein when the deflectable section is in the second configuration, the deflectable section has a curve.

2. The access device of claim 1, further comprising a handle, wherein the handle comprises a control, wherein the deflectable section is movable from the first configuration to the second configuration via the control, and wherein the deflectable section is movable from the second configuration to the first configuration via the control.

3. The access device of claim 2, further comprising a module, wherein the module is removably connectable to the handle, and wherein the module comprises an electronics module.

4. The access device of claim 3, wherein when the module is removably connected to the handle, the camera is powerable by a battery in the electronics module.

5. The access device of claim 4, wherein the module is reusable, and wherein the tube, the deflectable section, and/or the camera are disposable.

6. The access device of claim 5, wherein the deflectable section comprises a hinge, a first segment, and a second segment, wherein the first segment is rotatable relative to the second segment about the hinge, and wherein the second segment is rotatable relative to the first segment about the hinge.

7. The access device of claim 5, wherein the deflectable section and/or the camera are movable in a first direction and in a second direction via the control, and wherein the first direction is opposite the second direction.

8. The access device of claim 7, wherein the tube comprises a lumen, and wherein an object is advanceable and retractable in the lumen.

9. The access device of claim 8, wherein the lumen extends through the deflectable section and the camera.

10. The access device of claim 9, wherein the tube, the deflectable section, and the camera comprise an endoscope.

11. An access device comprising:
a first tube, wherein a stabilizer is advanceable out of the first tube and retractable into the first tube, wherein when the stabilizer is inside the first tube, the stabilizer has a contracted configuration, and wherein when the stabilizer is outside the first tube, the stabilizer has an expanded configuration; and
a second tube, wherein a third tube is advanceable out of the second tube and retractable into the second tube,
wherein the stabilizer is selectively advanceable and retractable independently of the third tube, wherein the third tube is selectively advanceable and retractable independently of the stabilizer, and
wherein when the stabilizer has the expanded configuration, the stabilizer has an opening that the third tube and/or an object is advanceable and/or retractable through.

12. The access device of claim 11, further comprising a connector, wherein the first tube and the second tube are removably connectable to the connector.

13. The access device of claim 12, wherein the first tube is connectable to a first mount and/or a second mount of the connector, and wherein the second tube is connectable to a third mount and/or a fourth mount of the connector.

14. The access device of claim 12, wherein the second tube is collapsible, wherein the second tube has an open configuration and a collapsed configuration, wherein when the second tube is in the open configuration, a lumen in the second tube is larger than when the second tube is in the collapsed configuration, and wherein when the second tube has the open configuration, the third tube is advanceable and retractable in the second tube.

15. The access device of claim 14, wherein when the second tube has the collapsed configuration, the third tube is advanceable in the lumen to open the second lumen.

16. The access device of claim 14, further comprising a handle having a first control, a second control, and third control, wherein the stabilizer is advanceable and retractable via the first control, wherein the third tube is advanceable and retractable via the second control, wherein the third tube comprises a third tube deflectable section, and wherein the third tube deflectable section is deflectable from a first configuration to a second configuration via the third control.

17. A system comprising:
a first device comprising the access device of claim 1; and
a second device comprising the access device of claim 11.

18. The system of claim 17, further comprising a connector, wherein the first device is removably attachable to the second device via the connector.

19. The system of claim 18, wherein the tube, the deflectable section, and/or the camera of the first device is/are removably attachable to the connector.

20. The system of claim 19, wherein when the first device and the second device are attached to the connector, the stabilizer is selectively advanceable and retractable independently of the advancement and retraction of the third tube, the stabilizer is selectively advanceable and retractable independently of the deflection of the third tube deflectable section, the stabilizer is selectively advanceable and retractable independently of the deflection of the deflectable section, the third tube is selectively advanceable and retractable independently of the advancement and retraction of the stabilizer, the third tube is selectively advanceable and retractable independently of the deflection of the third tube deflectable section, the third tube is selectively advanceable and retractable independently of the deflection of the deflectable section, the third tube deflectable section is selectively deflectable independently of the advancement and retraction of the stabilizer, the third tube deflectable section is selectively deflectable independently of the advancement and retraction of the third tube, the third tube deflectable section is selectively deflectable independently of the deflection of the deflectable section, the deflectable section is selectively deflectable independently of the advancement and retraction of the stabilizer, the deflectable section is selectively deflectable independently of the advancement and retraction of the third tube, and/or the deflectable section is selectively deflectable independently of the deflection the third tube deflectable section.
